(19) United States
(12) Patent Application Publication
(10) Pub. No.: US 2012/0071401 A1
(43) Pub. Date: Mar. 22, 2012

(54) AMYLIN AGONIST COMPOUNDS FOR ESTROGEN-DEFICIENT MAMMALS

(75) Inventors: Jonathan David Roth, San Diego, CA (US); James L. Trevaskis, San Diego, CA (US); David G. Parkes, San Diego, CA (US)

(73) Assignee: Amylin Pharamaceuticals, Inc., San Diego, CA (US)

(21) Appl. No.: 13/263,070

(22) PCT Filed: Apr. 9, 2010
(86) PCT No.: PCT/US10/30625

§ 371 Date: Nov. 29, 2011

Related U.S. Application Data
(60) Provisional application No. 61/168,317, filed on Apr. 10, 2009.

(51) Int. Cl. A61K 38/17; A61P 5/24; A61P 3/04; A61K 38/38

(52) U.S. Cl. 514/5.3; 514/10.2; 514/7.6

(57) ABSTRACT

Provided herein are methods to treat estrogen deficiency in mammals by administering amylin agonist compounds. Also provided herein are methods to treat obesity and overweight in estrogen-deficient mammals; methods to reduce or maintain body weight and/or body fat in estrogen-deficient mammals; and methods to increase Bdnf levels in mammals by administering effective amounts of amylin agonist compounds. The estrogen deficiency may be caused by menopause, perimenopause, post-menopause, ovarian dysfunction, an overectomy, a hysterectomy, and the like. The amylin agonist compounds may be any known in the art or described herein, such as pramlintide, davalintide, or SEQ ID NO: 142.

### CROSS-REFERENCE TO RELATED APPLICATIONS

This application claims benefit of U.S. provisional patent application Ser. 61/168,317, filed Apr. 10, 2009, the entire contents of which are incorporated herein for all purposes.

### FIELD

The disclosure is in the field of medicine, including the use of amylin agonist compounds to treat estrogen-deficient mammals.

### BACKGROUND

Menopause occurs when a woman stops ovulating and menstruation ceases. Estrogen is the female sex hormone responsible for ovulation. Estrogen has been found to influence body fat distribution. The results of a number of medical studies indicate that menopause is associated with a progressive increase in weight, and a redistribution of body fat to the abdominal region. Animal studies have shown that a lack of estrogen leads to excessive weight gain.

Women of childbearing age tend to store fat in the lower body while men and postmenopausal women store fat around the abdomen. Although being overweight is a risk factor alone, the redistribution of fat tissue further increases the risk of cardiovascular disease and diabetes. Postmenopausal women are at increased risk of coronary heart disease, partly because of the decline in estrogen production and concurrent elevations in total and low-density lipoprotein (LDL) cholesterol levels. Obesity, weight gain, and adverse changes in body fat distribution and composition are part of this phenomenon. Moreover, the rise in LDL cholesterol levels and the onset of other coronary heart disease risk factors (e.g., high blood pressure, high total cholesterol and triglyceride levels, insulin resistance) is directly influenced by weight gain. The greatest estrogen deficiencies occur when the ovaries have been removed or compromised by surgery or disease. Accordingly, many of these women experience significant weight problems. This appears to be related to the incurred deficiency of both testosterone and estrogen.

Hormone replacement therapy has been used to improve the quality of life for post-menopausal women to alleviate the symptoms associated with estrogen deficiency and to slow the progression of diseases associated with estrogen deficiency. A variety of hormone replacement therapies are available to help alleviate the deleterious physical and physiological changes associated with menopause. Such regimens include monotherapy, such as estrogen, and combination therapy, such as estradiol and progesterone/progestin.

For decades, menopausal and post-menopausal women were commonly treated with hormone replacement therapy. However, a large clinical trial called the Women's Health Initiative (WHI) reported that hormone replacement therapy can increase a woman's risk of breast cancer and heart disease. Writing Group for the Women's Health Initiative Investigators, JAMA, 288(3):321-333 (2002). Three years after the termination of the WHI, women who had received hormone replacement therapy during the clinical trial still had an increased risk for invasive breast cancer. Heiss et al, JAMA, 299(9):1036-1045 (2008). In view of the risks discussed in these reports, many doctors have stopped prescribing hormone replace therapy for their female patients, and many women have chosen to forgo hormone replacement therapy.

Estrogen is widely known to influence energy balance and body weight homeostasis through effects on feeding behavior and metabolism (1, 2). The absence of estrogen, for example, through experimental removal of the ovaries (OVX), leads to increased caloric consumption and subsequent accelerated body weight and fat mass gain (3, 4). In rodents, the induction of hyperphagia and rapid weight gain post-OVX can be reversed by estrogen replacement (3, 4). In addition to the well established effects of estrogen on the distribution of adipose tissue and metabolism, pharmacological and neurobiological studies comparing the responsiveness of intact and OVX rodents to neurohormonal signals have elucidated mechanisms whereby estrogen modulates the central control of energy balance. Acute studies implicate the presence of estrogen as enhancing the anorexigenic properties of short term signals of satiety (e.g., CCK (5, 6)), long term signals of adiposity (e.g., leptin, insulin (4, 7)), while decreasing the potency of orexigenic signals (e.g., melanin concentrating hormone, neuropeptide Y, ghrelin (8-11)). These pre-clinical findings have potential clinical significance, as many of the aforementioned neurohormonal signals have been, or are currently being pursued as potential anti-obesity targets, and post-menopausal women make up a high percentage of the obese population for whom these agents are ultimately prescribed (12).

One neurohormonal signal that has not been well explored in this regard is the pancreatic hormone amylin which has been touted as a potent satiety signal (13). Co-secreted with insulin in response to nutrient ingestion, amylin acts via its receptors in the area postrema (AP) of the brainstem to inhibit gastric emptying and food intake (14). Unlike CCK, however, sustained peripheral infusion of amylin to diet-induced obese (DIO) rats reduces food intake and body weight in a fat-specific manner (15). Administration of the synthetic amylin analog, pramlintide, to human subjects likewise reduced food intake and body weight (16, 17). Taken together these date indicate that amylin can exert both short- and long-term effects on energy balance. Given that estrogen can influence the efficacy/potency of a multitude of neurohormonal signals that have overarching consequences on body weight regulation.

Accordingly, there is a need in the art, inter alia, for new compounds and compositions that can be used without hormones, such as estrogen, to treat the weight gain, overweight, and obesity associated with menopause and other causes of estrogen deficiency.

### SUMMARY

Provided herein are methods of treating estrogen deficiency in mammals in need thereof by administering to the estrogen-deficient mammals therapeutically effective amounts of amylin agonist compounds or pharmaceutical compositions comprising amylin agonist compounds. Exemplary methods described herein include (i) treating obesity in estrogen-deficient mammals; (ii) treating overweight in estrogen-deficient mammals; (iii) reducing weight in estrogen-deficient mammals; (iv) reducing body fat in estrogen-deficient mammals; (v) reducing body fat while maintaining lean muscle mass in estrogen-deficient mammals; (vi) increasing satiety in estrogen-deficient mammals; (vii) reducing appetite in estrogen-deficient mammals; (viii) delaying gastric emptying in estrogen-deficient mammals; (ix) reducing gastric motility in estrogen-deficient mammals; (x) reducing ovariectomized weight gain and/or body fat in female mammals; (xi) treating ovariectomized obesity or overweight in female mammals; (xii) reducing menopausal weight gain and/or body fat in female humans; (xiii) reducing menopausal weight gain and/or body fat while maintaining lean muscle mass in female humans; (xiv) reducing postmenopausal weight gain and/or body fat in female humans; (xv) reducing postmenopausal weight gain and/or body fat while maintaining lean muscle mass in female humans; (xvi) reducing perimenopausal weight gain and/or body fat in female humans; (xvii) reducing postmenopausal weight gain and/or body fat while maintaining lean muscle mass in female humans; (xviii) treating menopausal obesity in female humans; (xix) treating perimenopausal obesity in female humans; (xx) treating postmenopausal obesity in female humans; (xxi) treating menopausal weight gain in female humans; (xxii) treating perimenopausal weight gain in female humans; (xxiii) treating postmenopausal weight gain in female humans; and (xxiv) increasing the levels of brain-derived neurotrophic factor (Bdnf) in estrogen-deficient mammals. The therapeutically effective amounts preferably provide at least a minimum therapeutically effective plasma level of the amylin agonist compounds in the mammals.

The methods described herein may further comprise improving glycemic control in the estrogen-deficient mammals by administering to the estrogen-deficient mammals therapeutically effective amounts of amylin agonist compounds or pharmaceutical compositions comprising amylin agonist compounds. Improving glycemic control includes lowering blood glucose levels, reducing hemoglobin A1c (HbA1c) levels, and the like. In one embodiment, the estrogen-deficient mammals have diabetes, such as type 2 diabetes, and are overweight or obese.

In other embodiments of the methods described herein, the estrogen-deficient mammals may be further administered an effective amount of leptin or a leptin analog. The leptin or leptin analog (such as metreleptin) can be administered in the same pharmaceutical composition as the amylin agonist compound or can be administered in a separate pharmaceutical composition.

In other embodiments of the methods described herein, the estrogen-deficient mammals may be further administered an effective amount of a GLP-1 receptor agonist or analog thereof (e.g., GLP-1(7-37) or an analog thereof; exendin-4 or an analog thereof); PYY or an analog thereof; GIP or an analog thereof. These compounds can be administered in the same pharmaceutical composition as the amylin agonist compound or can be administered in a separate pharmaceutical composition.

Other methods described herein include increasing levels of brain-derived neurotrophic factor (Bdnf) in a patient in need thereof by administering an amylin agonist compound. The patient can be a mammal, such as a human. The human may be male or female. The patient need not have an estrogen-deficiency for this method. The patient in need of increased levels of brain-derived neurotrophic factor may have WAGR syndrome.

In the methods described herein, the amylin agonist compounds may be any known in the art or described herein, such as ^{25,28,29}Pro-human-amylin (SEQ ID NO:20) or a pharmaceutically acceptable salt thereof, KCNTATCVLGRLSQELHRLQTYPRTNTGSNTY (SEQ ID NO:137) or a pharmaceutically acceptable salt thereof, and the like. The amylin agonist compounds may be in the form of a pharmaceutically acceptable salt and/or may be amidated.

### BRIEF DESCRIPTION OF THE FIGURES

FIGS. 1A-1C show the effects on body weight in three groups of diet-induced obese (DIO) female rats treated with rat amylin (SEQ ID NO: 15). Ovariectomized (OVX) female rats treated with amylin (FIG. 1B) exhibited a sustained weight loss that was about twice as great as that of intact female rats treated with amylin (FIG. 1A) and that of OVX female rats treated with amylin and estrogen (FIG. 1C). *p<0.05 vs. vehicle control.

FIGS. 2A-2C show the effects on food intake in three groups of DIO female rats treated with rat amylin (SEQ ID NO: 15). OVX female rats treated with amylin (FIG. 2B) exhibited a sustained reduction in food intake that was about twice as great as that of intact female rats treated with amylin (FIG. 2A) and that of OVX female rats treated with amylin and 17-β estradiol (FIG. 2C). *p<0.05 vs. vehicle control.

FIGS. 3A-3C shows the change in adiposity (FIGS. 3A and 3B) and change in percent lean mass (FIG. 3C) of DIO female rats treated with vehicle or with rat amylin (SEQ ID NO: 15). FIG. 3A depicts vehicle-corrected change in adiposity. OVX female rats treated with amylin exhibited a vehicle-corrected change in adiposity that was about 40% greater than that of intact female rats treated with amylin (Sham) and that of OVX female rats treated with amylin and estrogen (OVX+E). FIG. 3B depicts non-vehicle corrected change in adiposity dataset that is depicted in FIG. 3A. Filled bars=Amylin; unfilled bars=vehicle. *p<0.05 vs. vehicle control.

FIGS. 4A-4C show the effects of three additional, exemplary amylin agonists on body weight in SHAM-operated and OVX female rats. FIG. 4A depicts effects when the indicated animals are treated with a 50 μg/kg/day infusion of SEQ ID NO: 20. FIG. 4B depicts effects when the indicated animals are treated with a 2 μg/kg/day infusion of SEQ ID NO: 137. FIG. 4C depicts effects when the indicated animals are treated with a 5 μg/kg/day infusion of SEQ ID NO: 142. *p<0.05 vs. vehicle control.

FIGS. 5A-5F depict exaggerated amylin-induced changes in metabolism in estrogen-deficient DIO rats. Rate of oxygen consumption (FIGS. 5A and 5B), substrate utilization (FIGS. 5C and 5D) and locomotor activity in the X-axis (FIGS. 5E and 5F) are presented in either longitudinal form over ˜48 hrs (FIGS. 5A, 5C, and 5E) or represented as means during light and dark phases (FIGS. 5B, 5D, and 5F) for DIO sham-operated (SHAM) controls or ovariectomized (OVX) rats continuously infused with either vehicle or rat amylin (50 μg/kg/d). VO₂, rate of oxygen consumption, a marker of metabolic rate; RQ, respiratory quotient. Shaded grey areas indicate periods of darkness. To improve clarity of the graphs error bars were removed from data presented in longitudinal form. *p<0.05 vs. SHAM—vehicle; #p<0.05 vs. SHAM—amylin; ̂p<0.05 vs. OVX—vehicle.

FIG. 6A-6F indicates that reduced food consumption does not explain all of amylin's effects on body weight in OVX rats. Rate of oxygen consumption (FIGS. 6A and 6B), substrate utilization (FIGS. 6C and 6D) and locomotor activity in the X-axis (FIGS. 6E and 6F) are presented in either longitudinal form over ˜120 hrs (FIGS. 6A, 6C, and 6E) or represented as overall means (FIGS. 6B, 6D, and 6F) for DIO OVX rats continuously infused with either vehicle or rat amylin (50 μg/kg/d), or vehicle but restricted to the mean food intake of amylin-treated animals (yoked-fed). VO₂, rate of oxygen consumption, a marker of metabolic rate; RER, respiratory quotient. Shaded grey areas indicate periods of darkness. To improve clarity of the graphs error bars were removed from data presented in longitudinal form. *p<0.05 vs. OVX—vehicle; ̂p<0.05 vs. OVX—amylin.

FIGS. 7A-7J. BrdU staining in the area postrema (FIGS. 7A, 7C, 7E, and 7G; 10× magnification) or the nucleus of the solitary tract (FIGS. 7B, 7D, 7F, and 7H; 20× magnification) of OVX (FIGS. 7A, 7C, 7B, and 7D) and SHAM (FIGS. 7E, 7G, 7F, and 7H) animals continuously infused with either vehicle or rat amylin (50 μg/kg/d). Average cell counts in the AP (FIG. 71) or NTS (FIG. 7J). *p<0.05 vs.SHAM vehicle; ̂p<0.05 vs. SHAM amylin, SHAM vehicle and OVX vehicle. Counts of the NTS are unilateral.

FIGS. 8A-8C depict BrdU staining results in the hippocampus. FIG. 8A depicts BrdU staining with DAB in the hippocampus of an OVX/amylin-treated animal (10× magnification). FIG. 8B depicts average cell counts of the hippocampus with DAB staining of OVX and SHAM animals continuously infused with either vehicle or rat amylin (50 μg/kg/d); *p<0.05 vs.OVX vehicle. All cell counts of the hippocampus are unilateral. FIG. 8C depicts BrdU/NeuN staining in the hippocampus of an OVX/amylin-treated animal (20× magnification); BrdU label is green, NeuN label is red, colocalized cells appear yellow.

FIGS. 9A-9H depict enhancement of amylin-mediated body weight loss in estrogen-deficient ZDF rats. Percent vehicle-corrected (FIGS. 9A and 9B), or overall change in body weight (FIGS. 9C and 9D), cumulative food intake (FIGS. 9E and 9F), and change in body composition parameters (FIGS. 9G and 9H) of obese, diabetic sham-operated (SHAM) or ovariectomized (OVX) ZDF rats (FIGS. 9A, 9C, 9E, and 9G), or intact lean ZDF controls (FIGS. 9B, 9D, 9F, and 9H) continuously infused with either vehicle or rat amylin (50 μg/kg/d) for four weeks. *p<0.05 vs. SHAM—vehicle (or vs. vehicle controls for lean rats); #p<0.05 vs. SHAM—amylin; Ap<0.05 vs. OVX—vehicle.

### DETAILED DESCRIPTION

“Estrogen deficiency” refers to a mammal having less estrogen circulating in her blood than a typical healthy female of the same species at reproductive age. Estrogen deficiency may be caused by menopause, perimenopause, post-menopause, ovarian dysfunction, an ovariectomy, a hysterectomy, and the like. Estrogen deficiency may generally be diagnosed in a female human when estradiol levels are about 30 or below and follicle stimulating hormone (FSH) levels are about 30 or above. All post-menopausal female mammals are considered to have an estrogen deficiency. Perimenopausal female mammals may also have an estrogen deficiency caused by the fluctuation in estrogen levels and the cessation of ovarian function.

The term “estrogen” includes, for example, estradiols, estrones, estriols, and combinations of two or more thereof.

“Hormone replacement therapy” refers to the administration of hormones to estrogen-deficient mammals, such as menopausal or postmenopausal females. The hormones may include estrogen (including analogs and derivatives thereof), progesterone (including analogs and derivatives thereof), testosterone (including analogs and derivatives thereof), and combinations of two or more thereof. Hormone replacement therapy includes monotherapy and combination therapy. A mammal being administered a therapeutically effective amount of hormone replacement therapy would not be estrogen deficient.

“Obesity” and “overweight” refer to mammals having a weight that is greater than what they should have, and may be determined by, e.g., physical appearance, body mass index (BMI), waist-to-hip circumference ratios, skinfold thickness, and waist circumference. The Centers for Disease Control and Prevention define overweight as an adult human having a BMI of 25 to 29.9; and define obese as an adult human having a BMI of 30 or higher. BMI is a calculation based on a person's sex, weight, and height. The Centers for Disease Control and Prevention state that a person with a waist-to-hip ratio greater than 1.0 is overweight.

“Lean body mass” or “lean mass” refers to the fat-free mass of the body, i.e., total body weight minus body fat weight is lean body mass. Lean body mass can be measured by methods such as hydrostatic weighing, computerized chambers, dual-energy X-ray absorptiometry, skin calipers, magnetic resonance imaging (MRI) and bioelectric impedance analysis (BIA).

By “body fat”, or “whole body fat” is meant deposited lipid or lipids as it may occur or be found throughout the body.

By “fat distribution”, “body fat distribution”, or “whole body fat distribution” is meant the location of fat deposits in the body. Such locations of fat deposition include, for example, subcutaneous, visceral and ectopic fat depots.

By “subcutaneous fat” is meant the deposit of lipids just below the skin's surface. The amount of subcutaneous fat in an estrogen-deficient mammal can be measured using any method available for the measurement of subcutaneous fat. Methods of measuring subcutaneous fat are known in the art, for example, those described in U.S. Pat. No. 6,530,886, the entirety of which is incorporated herein by reference.

By “visceral fat” is meant the deposit of fat as intra-abdominal adipose tissue. Visceral fat surrounds vital organs and can be metabolized by the liver to produce blood cholesterol. Visceral fat has been associated with increased risks of conditions such as polycystic ovary syndrome, metabolic syndrome and cardiovascular diseases.

By “ectopic fat storage” is meant lipid deposits within and around tissues and organs that constitute the lean body mass (e.g., skeletal muscle, heart, liver, pancreas, kidneys, blood vessels). Generally, ectopic fat storage is an accumulation of lipids outside classical adipose tissue depots in the body.

“Mammal” refers to warm-blooded animals that generally have fur or hair, that give live birth to their progeny, and that feed their progeny with milk. Mammals include female humans; companion animals (e.g., dogs, cats); farm animals (e.g., cows, horses, sheep, pigs, goats); wild animals; and the like. In one embodiment, the mammal is a female. In one embodiment, the mammal is a female human. In one embodiment, the mammal is a cat or dog. In one embodiment, the mammal is a diabetic mammal, e.g., a female human having type 2 diabetes. In one embodiment, the mammal is an obese diabetic mammal, e.g., an obese female human having type 2 diabetes.

“Reduced nutrient availability” is meant to include any means by which the body reduces the nutrients available to the body to store as fat. In other words, reducing nutrient availability may be by means that include, but are not limited to, reducing appetite, increasing satiety, affecting food choice/taste aversion, increasing metabolism, and/or decreasing or inhibiting food absorption. Exemplary mechanisms that may be affected include delayed gastric emptying or decreased absorption of food in the intestines.

“Increased nutrient availability” is meant to include any means by which the body increases the nutrients available to the body to store as fat. In other words, increasing nutrient availability may be by means that include, but are not limited to, increasing appetite, decreasing satiety, affecting food choice, decreasing taste aversion, decreasing metabolism, and/or increasing food absorption. Exemplary mechanisms that may be affected include decreasing gastric hypomotility or increasing absorption of food in the intestines.

“Amylin agonist compounds” include native amylin peptides, amylin analog peptides, and other compounds (e.g., small molecules) that have amylin agonist activity. The “amylin agonist compounds” can be derived from natural sources, can be synthetic, or can be derived from recombinant DNA techniques. Amylin agonist compounds have amylin agonist receptor binding activity and may comprise amino acids (e.g., natural, unnatural, or a combination thereof), peptide mimetics, chemical moieties, and the like. The skilled artisan will recognize amylin agonist compounds using amylin receptor binding assays or by measuring amylin agonist activity in soleus muscle assays. In one embodiment, amylin agonist compounds will have an IC₅₀ of about 200 or less, about 100 or less, or about 50 or less, in an amylin receptor binding assay, such as that described herein, in U.S. Pat. No. 5,686,411, and US Publication No. 2008/0176804, the disclosures of which are incorporated by reference herein. In one embodiment, amylin agonist compounds will have an EC₅₀ of about 20 or less, about 15 or less, about 10 or less, or about 5 or less in a soleus muscle assay, such as that described herein and in U.S. Pat. No. 5,686,411, the disclosure of which is incorporated by reference herein. In one embodiment, the amylin agonist compound has at least 90% or 100% sequence identity to ^{25,28,29}Pro-human-amylin (SEQ ID NO:20). In one embodiment, the amylin agonist compound is a peptide chimera of amylin (e.g., human amylin (SEQ ID NO:1), rat amylin (SEQ ID NO:15), and the like) and calcitonin (e.g., human calcitonin (SEQ ID NO:140), salmon calcitonin (SEQ ID NO:141), and the like). In one embodiment, the amylin agonist compound has at least 90% or 100% sequence identity to SEQ ID NO: 137. Suitable and exemplary amylin agonist compounds are also described in US Publication No. 2008/0274952, the disclosure of which is incorporated by reference herein in its entirety.

“Analog” as used herein refers to a compound that has properties that are as good as or better than the parent compound. The analog may have superior stability, solubility, efficacy, half-life, and the like. In one embodiment, an analog is a compound that has at least 75% sequence identity to the parent compound, at least 80% sequence identity, at least 85% sequence identity, at least 90% sequence identity, or at least 95% sequence identity to the parent compound. For example, a leptin analog may have at least 75% sequence identity to human leptin; an exendin analog may have at least 75% sequence identity to exendin-4; a GLP-1(7-37) analog may have at least 75% sequence identity to GLP-1(7-37), and the like.

The nomenclature for the compounds described herein is used to indicate (1) the peptide that the amino acid sequence is based on and (2) the modifications that have been made to that amino acid sequence. An amino acid preceded by a superscript number indicates that the named amino acid replaces the amino acid normally present at that particular amino acid position in the amino acid sequence. For example, ¹⁸Arg^{25,28}Pro-h-amylin (SEQ ID NO: 25) refers to a peptide based on the amino acid sequence of human amylin (i.e., h-amylin) and which has the following substitutions: Arg replaces His at position 18 in h-amylin; Pro replaces Ala at position 25 in h-amylin; and Pro replaces Ser at position 28 in h-amylin. The term des-¹Lys-h-amylin (SEQ ID NO: 2) refers to a peptide based on the amino acid sequence of human amylin (i.e., h-amylin) except that the Lys at position 1 (i.e., ¹Lys) in h-amylin is deleted (i.e., des-) from the amino acid sequence.

In one embodiment, the amylin agonist peptide is a compound of Formula (I) or a pharmaceutically acceptable salt thereof:

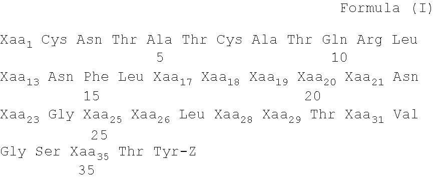

wherein: ²Cys and ⁷Cys form a disulfide bond; Xaa₁ is Lys or hydrogen; Xaa₁₃ is Ala or Thr; Xaa₁₇ is Ile or Val; Xaa₁₈ is Arg or His; Xaa₁₉ is Thr or Ser; Xaa₂₁ is His or Asn; Xaa₂₀ is Asn or Ser; Xaa₂₃ is Leu or Phe; Xaa₂₅ is Pro, Ala, or Thr; Xaa₂₆ is Ile, Val, or Ala; Xaa₂₈ is Pro, Ser, or Leu; Xaa₂₉ is Pro or Ser; Xaa₃₁ is Asp or Asn; Xaa₃₅ is Asn or Asp; and Z is OH or NH₂.

In one embodiment for the compounds of Formula (I), at least one of Xaa₂₅, Xaa₂₈, and Xaa₂₉ is proline. In one embodiment for the compounds of Formula (I), at least two of Xaa₂₅, Xaa₂₈, and Xaa₂₉ are proline.

Exemplary compounds of Formula (I) include human amylin (SEQ ID NO: 1); des-¹Lys-h-amylin (SEQ ID NO: 2); ¹⁷Ile¹⁸Arg²³Leu-h-amylin (SEQ ID NO: 3); ¹⁷Ile¹⁸Arg²³Leu²⁶Val²⁹Pro-h-amylin (SEQ ID NO: 4); ²⁵Pro-h-amylin (SEQ ID NO: 5) amylin (SEQ ID NO: 6); ²⁹Pro-h-amylin (SEQ ID NO: 7); ¹³Thr¹⁸Arg²¹His²³Leu²⁶Ala²⁹Pro³¹Asp-h-amylin (SEQ ID NO: 8); ¹³Thr²¹His²³Leu²⁶Ala²⁸Leu²⁹Pro³¹Asp-h-amylin (SEQ ID NO: 9): ¹³Thr²¹His²³Leu²⁶Ala²⁹Pro³¹Asp-h-amylin (SEQ ID NO: 10); des-¹Lys-¹³Thr²¹His²³Leu²⁶Ala²⁹Pro³¹Asp-h-amylin (SEQ ID NO: 11); and pharmaceutically acceptable salts of any of these peptides.

Other exemplary compounds of Formula (I) include monkey amylin (SEQ ID NO: 12); cat amylin (SEQ ID NO: 13); dog amylin (SEQ ID NO: 14); rat amylin (SEQ ID NO: 15); mouse amylin (SEQ ID NO: 16); hamster amylin SEQ ID NO: 17); guinea pig amylin (SEQ ID NO: 18); degu amylin (SEQ ID NO: 19); and pharmaceutically acceptable salts of any of these compounds.

In one embodiment, the amylin agonist peptide is a compound of Formula (II) or a pharmaceutically acceptable salt thereof:

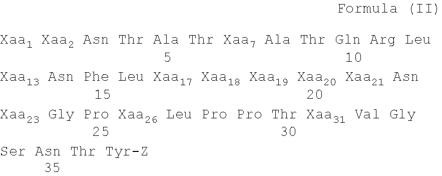

wherein: Xaa₁ is Lys, Ala, Ser, or hydrogen (preferably Lys or hydrogen); Xaa₂ is Ser, Asp, Glu, Lys, ornithine (Orn), or Cys, wherein the amino acid is optionally linked to the amino acid at Xaa₇ to form an intramolecular linkage (e.g., Cys forming a disulfide bond with the Cys at Xaa₇; Ser forming a bond with Ser at Xaa₇; Asp or Lys forming a bond with Lys or Asp, respectively, at Xaa₇); Xaa₇ is Ser, Asp, Glu, Lys, Orn, or Cys, wherein the amino acid is optionally linked to the amino acid at Xaa₂ to form an intramolecular linkage (e.g., Cys forming a disulfide bond with the Cys at Xaa₂; Ser forming a bond with Ser at Xaa₂; Asp or Lys forming a bond with Lys or Asp, respectively, at Xaa₂); Xaaₙ is Ala, Ser, or Thr (preferably Ala or Thr); Xaaᵣ is Val, Leu, or Ile (preferably Val or Ile); Xaa₁₈ is His or Arg; Xaa₁₉ is Ser or Thr (preferably Ser); Xaa₂₀ is Ser, Thr, Gln, or Asn (preferably Ser or Asn); Xaa₂₁ is Asn, Gln, or His (preferably Asn or His); Xaa₂₃ is Phe, Leu, or Tyr (preferably Phe or Leu); Xaa₂₆ is Ile, Val, Ala, or Leu (preferably Ile, Val, or Ala; more preferably Ile or Val); Xaa₃₁ is Asn, Asp, or Gln (preferably Asn or Asp); and Z is OH or NH₂ (preferably NH₂).

In one embodiment, Xaa₂ and Xaa ₇ may form an intramolecular linkage such as disulfide bond; amide bond; alkyl acids and alkyl amines which may form cyclic lactams; alkyl aldehydes or alkyl halides and alkylamines which may condensed and be reduced to form an alkyl amine or imine bridge; or side chains which may be connected to form an alkyl, alkenyl, alkynyl, ether or thioether bond. Alkyl chains may include lower alkyl groups having from about 1 to about 6 carbon atoms. In certain embodiments, the intramolecular linkage may be a disulfide, amide, imine, amine, alkyl and alkene bond. In certain embodiments, Xaa₂ and Xaa ₇ are independently selected from Ser, Asp, Glu, Lys, Orn, or Cys. In certain embodiments, Xaa₂ and Xaa₇ are Cys and Cys. In other embodiments, Xaa₂ and Xaa₇ are Ser and Ser. In still other embodiments, Xaa₂ and Xaa₇ are Asp and Lys or Lys and Asp. In one embodiment, the amino acid residues at Xaa₂ and Xaa₇ do not form an intramolecular linkage.

In one embodiment, the amylin agonist compound of Formula (II) is ^{25,28,29}Pro-h-amylin having the formula: KCNTATCATQRLANFLVHSSNNFGPILPPTNVGSNTY-NH₂ (SEQ ID NO:20), where ²Cys and ⁷Cys form a disulfide bond; or a pharmaceutically acceptable salt thereof. In one embodiment, the compound is an acetate salt of ^{25,28,29}Pro-h-amylin.

Other exemplary compounds of Formula (II) include: des-¹Lys-^{25,28,29}Pro-h-amylin (SEQ ID NO: 21); ¹⁸Arg^{25,28,29}Pro-h-amylin (SEQ ID NO: 22); des-¹Lys-¹⁸Arg^{25,28,29}Pro-h-amylin (SEQ ID NO: 23); ²⁵Pro²⁶Val^{28,29}Pro-h-amylin (SEQ ID NO: 24); ¹⁷Ile^{25,28,29}Pro-h-amylin (SEQ ID NO: 25); ²³Leu²⁵Pro²⁶Val^{28,29}Pro-h-amylin (SEQ ID NO: 26); ¹⁸Arg²³Leu^{25,28,29}Pro-h-amylin (SEQ ID NO: 27); ¹⁷Ile²³Leu^{25,28,29}Pro-h-amylin (SEQ ID NO: 28); des-¹Lys-¹⁷Ile^{25,28,29}Pro-h-amylin (SEQ ID NO: 29); ¹⁷Ile¹⁸Arg²³Leu²⁵Pro²⁶Val^{28,29}Pro-h-amylin (SEQ ID NO: 30); ¹³Thr¹⁸Arg²¹His²³Leu²⁵Pro²⁶Ala^{28,29}Pro³¹Asp-h-amylin (SEQ ID NO: 31); and pharmaceutically acceptable salts of these peptides. The skilled artisan will recognize that these exemplary compounds may optionally have an intramolecular disulfide linkage between the Cys amino acid residues at positions 2 and 7.

In one embodiment, the amylin agonist peptide is a compound of Formula (III) or a pharmaceutically acceptable salt thereof:

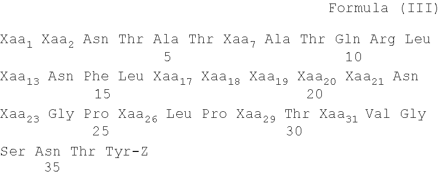

wherein: Xaa₁ is Lys, Ala, Ser, or hydrogen (preferably Lys or hydrogen); Xaa₂ is Ser, Asp, Glu, Lys, Orn, or Cys, wherein the amino acid is optionally linked to the amino acid at Xaa₇ to form an intramolecular linkage (e.g., Cys forming a disulfide bond with Cys at Xaa₇; Ser forming a bond with Ser at Xaa₇; Asp or Lys forming a bond with Lys or Asp, respectively, at Xaa₇); Xaa₇ is Ser, Asp, Glu, Lys, Orn, or Cys, wherein the amino acid is optionally linked to the amino acid at Xaa₂ to form an intramolecular linkage (e.g., Cys forming a disulfide bond with Cys at Xaa₂; Ser forming a bond with Ser at Xaa₂; Asp or Lys forming a bond with Lys or Asp, respectively, at Xaa₂); Xaa₁₃ is Ala, Ser, or Thr (preferably Thr or Ala; more preferably Ala); Xaa₁₇ is Val, Leu, or Ile (preferably Val or Ile); Xaa₁₈ is His or Arg; Xaa₁₉ is Ser or Thr (preferably Ser); Xaa₂₀ is Ser, Thr, Gln, or Asn (preferably Ser or Asn; more preferably Ser); Xaa₂₁ is Asn, Gln, or His (preferably Asn or His; more preferably Asn); Xaa₂₃ is Phe, Leu, or Tyr (preferably Phe or Leu); Xaa₂₆ is Ile, Val, Ala, or Leu (preferably Val or Ile); Xaa₂₉ is Ser or Thr;

Xaa₃₁ is Asn, Asp, or Gln (preferably Asn or Asp); and Z is OH or NH₂ (preferably NH₂).

In one embodiment, Xaa₂ and Xaa₇ may form an intramolecular linkage such as disulfide bond; amide bond; alkyl acids and alkyl amines which may form cyclic lactams; alkyl aldehydes or alkyl halides and alkylamines which may condensed and be reduced to form an alkyl amine or imine bridge; or side chains which may be connected to form an alkyl, alkenyl, alkynyl, ether or thioether bond. Alkyl chains may include lower alkyl groups having from about 1 to about 6 carbon atoms. In certain embodiments, the intramolecular linkage may be a disulfide, amide, imine, amine, alkyl and alkene bond. In certain embodiments, Xaa₂ and Xaa₇ are independently selected from Ser, Asp, Glu, Lys, Ornithine, or Cys. In certain embodiments, Xaa₂ and Xaa₇ are Cys and Cys. In other embodiments, Xaa₂ and Xaa₇ are Ser and Ser. In still other embodiments, Xaa₂ and Xaa₇ are Asp and Lys or Lys and Asp. In one embodiment, the amino acid residues at Xaa₂ and Xaa₇ do not form an intramolecular linkage.

Exemplary compounds of Formula (III) include: ¹⁸Arg^{25,28}Pro-h-amylin (SEQ ID NO: 32); des-'Lys-¹⁸Arg^{25,28}Pro-h-amylin (SEQ ID NO: 33); ¹⁸Arg²³Leu^{25,28}Pro-h-amylin (SEQ ID NO: 34); ²³Leu²⁵Pro²⁶Val²⁸Pro-h-amylin (SEQ ID NO: 35); des-¹Lys-²³Leu²⁵Pro²⁶Val²⁸Pro-h-amylin (SEQ ID NO: 36); ¹⁸Arg²³Leu²⁵Pro²⁶Val²⁸Pro-h-amylin (SEQ ID NO: 37); 25,28Pro-h-amylin (SEQ ID NO: 38); and pharmaceutically acceptable salts of these peptides.

In one embodiment, the amylin agonist peptide is a compound of Formula (IV) or a pharmaceutically acceptable salt thereof:

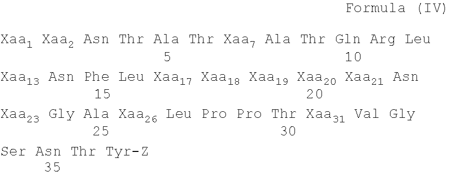

wherein: Xaa₁ is Lys, Ala, Ser, or hydrogen (preferably Lys or hydrogen); Xaa₂ is Ser, Asp, Glu, Lys, Orn, or Cys, wherein the amino acid is optionally linked to the amino acid at Xaa₇ to form an intramolecular linkage (e.g., Cys forming a disulfide bond with Cys at Xaa₇; Ser forming a bond with Ser at Xaa₇; Asp or Lys forming a bond with Lys or Asp, respectively, at Xaa₇); Xaa₇ is Ser, Asp, Glu, Lys, Orn, or Cys, wherein the amino acid is optionally linked to the amino acid at Xaa₂ to form an intramolecular linkage (e.g., Cys forming a disulfide bond with Cys at Xaa₂; Ser forming a bond with Ser at Xaa₂; Asp or Lys forming a bond with Lys or Asp, respectively, at Xaa₂); Xaa₁₃ is Ala, Ser, or Thr (preferably Thr or Ala; more preferably Ala); Xaa₁₇ is Val, Leu, or Ile (preferably Val or Ile); Xaa₁₈ is His or Arg; Xaa₁₉ is Ser or Thr (preferably Ser);Xaa₂₀ is Ser, Thr, Gln, or Asn (preferably Ser or Asn; more preferably Ser); Xaa₂₁ is Asn, Gln, or His (preferably Asn or His; more preferably Asn); Xaa₂₃ is Phe, Leu, or Tyr (preferably Phe or Leu); Xaa₂₆ is Ile, Val, Ala, or Leu (preferably Val, Ile, or Ala; more preferably Val or Ile); Xaa₃₁ is Asn, Asp, or Gln (preferably Asn or Asp; more preferably Asn); and Z is OH or NH₂ (preferably NH₂).

In one embodiment, Xaa₂ and Xaa₇ may form an intramolecular linkage such as disulfide bond; amide bond; alkyl acids and alkyl amines which may form cyclic lactams; alkyl aldehydes or alkyl halides and alkylamines which may condensed and be reduced to form an alkyl amine or imine bridge; or side chains which may be connected to form an alkyl, alkenyl, alkynyl, ether or thioether bond. Alkyl chains may include lower alkyl groups having from about 1 to about 6 carbon atoms. In certain embodiments, the intramolecular linkage may be a disulfide, amide, imine, amine, alkyl and alkene bond. In certain embodiments, Xaa₂ and Xaa₇ are independently selected from Ser, Asp, Glu, Lys, Orn, or Cys. In certain embodiments, Xaa₂ and Xaa₇ are Cys and Cys. In other embodiments, Xaa₂ and Xaa₇ are Ser and Ser. In still other embodiments, Xaa₂ and Xaa₇ are Asp and Lys or Lys and Asp. In one embodiment, the amino acid residues at Xaa₂ and Xaa₇ do not form an intramolecular linkage.

Exemplary compounds of Formula (IV) include ¹³Thr¹⁸Arg²¹His²³Leu^{28,29}Pro³¹Asp-h-amylin (SEQ ID NO: 39), ^{28,29}Pro-h-amylin (SEQ ID NO:138), and pharmaceutically acceptable salts thereof. The skilled artisan will recognize that these exemplary compounds have an intramolecular disulfide linkage between the Cys amino acid residues at positions 2 and 7.

In one embodiment, the amylin agonist peptide is a compound of Formula (V) or a pharmaceutically acceptable salt thereof:

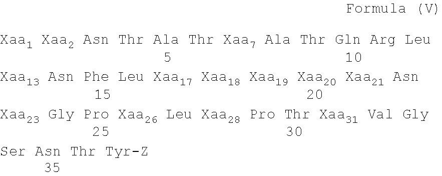

wherein: Xaa₁ is Lys, Ala, Ser, or hydrogen (preferably Lys or hydrogen); Xaa₂ is Ser, Asp, Glu, Lys, Orn, or Cys, wherein the amino acid is optionally linked to the amino acid at Xaa₇ to form an intramolecular linkage (e.g., Cys forming a disulfide bond with Cys at Xaa₇; Ser forming a bond with Ser at Xaa₇; Asp or Lys forming a bond with Lys or Asp, respectively, at Xaa₇); Xaa₇ is Ser, Asp, Glu, Lys, Orn, or Cys, wherein the amino acid is optionally linked to the amino acid at Xaa₂ to form an intramolecular linkage (e.g., Cys forming a disulfide bond with Cys at Xaa₂; Ser forming a bond with Ser at Xaa₂; Asp or Lys forming a bond with Lys or Asp, respectively, at Xaa₂); Xaa₁₃ is Ala, Ser, or Thr (preferably Thr or Ala; more preferably Ala); Xaa₁₇ is Val, Leu, or Ile (preferably Val or Ile); Xaa₁₈ is His or Arg; Xaa₁₉ is Ser or Thr (preferably Ser); Xaa₂₀ is Ser, Thr, Gln, or Asn (preferably Ser or Asn); Xaa₂₁ is Asn, Gln, or His (preferably Asn or His; more preferably Asn); Xaa₂₃ is Phe, Leu, or Tyr (preferably Phe or Leu); Xaa₂₆ is Ile, Val, Ala, or Leu (preferably Val, Ile, or Ala; more preferably Val or Ile); Xaa₂₈ is Ser or Thr; Xaa₃₁ is Asn, Asp, or Gln (preferably Asn or Asp; more preferably Asn); and Z is OH or NH₂ (preferably NH₂).

In one embodiment, Xaa₂ and Xaa₇ may form an intramolecular linkage such as disulfide bond; amide bond; alkyl acids and alkyl amines which may form cyclic lactams; alkyl aldehydes or alkyl halides and alkylamines which may condensed and be reduced to form an alkyl amine or imine bridge; or side chains which may be connected to form an alkyl, alkenyl, alkynyl, ether or thioether bond. Alkyl chains may include lower alkyl groups having from about 1 to about 6 carbon atoms. In certain embodiments, the intramolecular linkage may be a disulfide, amide, imine, amine, alkyl and alkene bond. In certain embodiments, Xaa₂ and Xaa₇ are independently selected from Ser, Asp, Glu, Lys, Orn, or Cys. In certain embodiments, Xaa₂ and Xaa₇ are Cys and Cys. In other embodiments, Xaa₂ and Xaa₇ are Ser and Ser. In still other embodiments, Xaa₂ and Xaa₇ are Asp and Lys or Lys and Asp. In one embodiment, the amino acid residues at Xaa₂ and Xaa₇ do not form an intramolecular linkage.

In one embodiment, the amylin agonist compound of Formula (V) is ^{25,29}Pro-h-amylin (SEQ ID NO: 139) or a pharmaceutically acceptable salt thereof.

In one embodiment, the amylin agonist peptide is a compound of Formula (VI) or a pharmaceutically acceptable salt thereof:

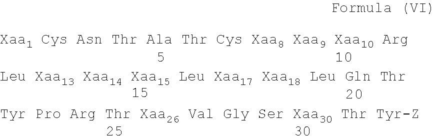

wherein: ²Cys and ⁷Cys form a disulfide bond; Xaa₁ is Lys, Ser, or absent; Xaa₈ is Ala or Val; Xaa₉ is Leu or Thr; Xaa₁₀ is Gln or Gly; Xaa₁₃ is Ala, Thr, or Ser; Xaa₁₄ is Asn or Gln; Xaa₁₅ is Phe or Glu; Xaa₁₇ is Ile, Val, or His; Xaa₁₈ is Arg or His; Xaa₂₆ is Asp, Asn, or Thr; Xaa₃₀ is Asn or Asp; and Z is OH or NH₂.

Exemplary compounds of Formula (VI) are SEQ ID NOs:40-137. In one embodiment, the compound of Formula (VII) is: KCNTATCVLGRLSQELHRLQTYPATNTGSNTY (SEQ ID NO: 137), which may optionally be amidated, or a pharmaceutically acceptable salt thereof.

In one embodiment, the amylin agonist peptide has at least 87% sequence identity to the amino acid sequence of SEQ ID NO: 137. Such amylin agonist peptides include SEQ ID NOs: 51, 52, 76, 88, and 117. In one embodiment, the amylin agonist peptide has at least 90% sequence identity to the amino acid sequence of SEQ ID NO:137. Such amylin agonist peptides include SEQ ID NOs: 44, 45, 49, 58, 66, 71, 75, 86, 108, 110, 113, 115, 116, 120, 122, 123, 124, 131, or 132. In one embodiment, the amylin agonist peptide has at least 93% sequence identity to the amino acid sequence of SEQ ID NO:137. Such amylin agonist peptides include SEQ ID NOs: 55, 67, 70, 73, 82, 83, 84, 89, 94, 100, 103, 106, 107, 111, 112, 125, and 133. In one embodiment, the amylin agonist peptide has at least 96% sequence identity to the amino acid sequence of SEQ ID NO:137. Such amylin agonist peptides include SEQ ID NOs: 40, 42, 43, 46, 47, 48, 54, 64, 65, 68, 69, 74, 78, 79, 80, 81, 85, 90, 91, 92, 93, 95, 96, 97, 98, 99, 101, 102, 109, 118, 119, 121, 130, and 137.

In one embodiment, the amylin agonist peptide may be any one of SEQ ID NOs: 40-137 or a pharmaceutically acceptable salt thereof; or an amylin agonist peptide having at least 87%, 90%, 93%, or 96% sequence identity to the amino acid sequence of any one of SEQ ID NOs: 40-137 or a pharmaceutically acceptable salt thereof. The amylin agonist peptide may optionally be amidated.

The amylin agonist peptides of SEQ ID NOs:40-137 are:

SEQ ID NO: 40
KCNTATCVLGKLSQELHRLQTYPRTNTGSNTY
SEQ ID NO: 41
KCNTATCVLGRLSQELHRLQTLPRTNTGSNTY
SEQ ID NO: 42
KCNTATCVLGRLSQELHRLQTYPPTNTGSNTY
SEQ ID NO: 43
KCNTATCVLGRLSQELHRLQTYPRTNVGSNTY
SEQ ID NO: 44
KCNTATCVLGRLSQELHRLQTLPPTNVGSNTY
SEQ ID NO: 45
KCNTATCVLGRLANFLHRLQTYPRTNTGSNTY
SEQ ID NO: 46
ACNTATCVLGRLSQELHRLQTYPRTNTGSNTY
SEQ ID NO: 47
KCNAATCVLGRLSQELHRLQTYPRTNTGSNTY
SEQ ID NO: 48
KCNTAACVLGRLSQELHRLQTYPRTNTGSNTY
SEQ ID NO: 49
CANLSTCVLGRLSQELHRLQTYPRTNTGSNTY
SEQ ID NO: 50
isocaproyl-STAVLGRLSQELHRLQTYPRTNTGSNTY
SEQ ID NO: 51
CSNASTCVLGRLSQELHRLQTYPRTNTGSNTY
SEQ ID NO: 52
CSNLATCVLGRLSQELHRLQTYPRTNTGSNTY
SEQ ID NO: 53
CSNLSACVLGRLSQELHRLQTYPRTNTGSNTY
SEQ ID NO: 54
KCNTATCVLGRLSQELHKLQTYPRTNTGSNTY
SEQ ID NO: 55
KCNTATCVLGRLSQELHRLQTYPRTNTGSGTP
SEQ ID NO: 56
CSALSTCVLGRLSQELHRLQTYPRTNTGSNTY
SEQ ID NO: 57
Ac-(Agy)SNLST(Agy)VLGRLSQELHRLQTYPRTNTGSNTY
SEQ ID NO: 58
Ac-K(Agy)NTAT(Agy)VLGRLSQELHRLQTYPRTNTGSNTY
SEQ ID NO: 59
Isocaproyl-STAVL(Aib)RLSQELRLQTYPRTNTGSGTP
SEQ ID NO: 60
Isocaproyl-STAVLG[K(For)]LSQELH[K(For)]LQTYPRTNTGS
GTP
SEQ ID NO: 61
Isocaproyl-STAVL(Aib)[K(For)]LSQEL(Aib)[K(For)]LQT
YPRTNTGSNTY
SEQ ID NO: 62
Isocaproyl-STAVL(Aib)[K(For)]LSQEL(Aib)[K(For)]LQT
YPRTNVGSNTY
SEQ ID NO: 63
KCNTATCLLQQLQKLLQKLKQYPRTNTGSNTY
SEQ ID NO: 64
KCNTASCVLGRLSQELHRLQTYPRTNTGSNTY
SEQ ID NO: 65
KCNTAVCVLGRLSQELHRLQTYPRTNTGSNTY
SEQ ID NO: 66
KCNTATCVLGRLSQELHRYPRTNTGSNTY
SEQ ID NO: 67
KCNTATCVLGK(For)LSQELHK(For)LQTYPRTNTGSNTY
SEQ ID NO: 68
KCNTA(d-Thr)CVLGRLSQELHRLQTYPRTNTGSNTY
SEQ ID NO: 69
KCNTA(dAh)CVLGRLSQELHRLQTYPRTNTGSNTY
SEQ ID NO: 70
Ac-ACNTATCVLGRLSQELHK(PEG5000)LQTYPRTNTGSNTY
SEQ ID NO: 71
KCNTATCVLGRLSQELHRLQTLQTYPRTNTGSNTY
SEQ ID NO: 72
KCNTATCVLGRLSQELHRLQTLLQTYPRTNTGSNTY
SEQ ID NO: 73
KCNTATCVLGKLSQELHKLQTYPRTNTGSNTY
SEQ ID NO: 74
KCNTSTCVLGRLSQELHRLQTYPRTNTGSNTY
SEQ ID NO: 75
KCNTATCATQRLSQELHRLQTYPRTNTGSNTY
SEQ ID NO: 76
KCNTATCATQRLSQELHRLQTYPRTNVGSNTY
SEQ ID NO: 77
KCNTSTCATQRLANELVRLQTYPRTNVGSNTY
SEQ ID NO: 78
KCNTA(Hse)CVLGRLSQELHRLQTYPRTNTGSNTY
SEQ ID NO: 79
KCNTA(Ahb)CVLGRLSQELHRLQTYPRTNTGSNTY
SEQ ID NO: 80
KCNTA(Ahp)CVLGRLSQELHRLQTYPRTNTGSNTY
SEQ ID NO: 81
KCNTAT(OPO3H2)CVLGRLSQELHRLQTYPRTNTGSNTY
SEQ ID NO: 82
KCNTATCVLG(Orn)LSQELH(Orn)LQTYPRTNTGSNTY
SEQ ID NO: 83
KCNTATCVLG(Cit)LSQELH(Cit)LQTYPRTNTGSNTY
SEQ ID NO: 84
KCNTATCVLG(homoK)LSQELH(homoK)LQTYPRTNTGSNTY
SEQ ID NO: 85
L-OctylglycineKCNTATCVLGRLSQELHRLQTYPRTNTGSNTY
SEQ ID NO: 86
N-3,6-dioxaoctanoyl-CNTATCVLGRLSQELHRLQTVPRTNTGSNT
Y
SEQ ID NO: 87
KCNTATCMLGRYTQDFHRLQTYPRTNTGSNTY
SEQ ID NO: 88
DSNLSTKVLGRLSQELHRLQTYPRTNTGSNTY
SEQ ID NO: 89
KDNTATKVLGRLSQELHRLQTYPRTNTGSNTY
SEQ ID NO: 90
CNTATCVLGRLSQELHRLQTYPRTNTGSNTY
SEQ ID NO: 91
KCNTATCVLGRLSQELHRLQTYPRTNTGSNTY(9Anc)
SEQ ID NO: 92
KCNTATCVLGRLSQELHRLQTYPRTNTGSNTY(L-octylglycine)
SEQ ID NO: 93
N-isocaproyl-KCNTATCVLGRLSQELHRLQTYPRTNTGSNTY
SEQ ID NO: 94
KCNTATCVLG(homoR)LSQELH(homoR)LQTYPRTNTGSNTY
SEQ ID NO: 95
FCNTATCVLGRLSQELHRLQTYPRTNTGSNTY
SEQ ID NO: 96
KCNTATCVLGRLSQELH(Cit)LQTYPRTNTGSNTY
SEQ ID NO: 97
KCNTATCVLGRLSQELH(Orn)LQTYPRTNTGSNTY
SEQ ID NO: 98
ICNTATCVLGRLSQELHRLQTYPRTNTGSNTY
SEQ ID NO: 99
1-Octylglycine-CNTATCVLGRLSQELHRLQTYPRTNTGSNTY
SEQ ID NO: 100
Isocaproyl-CNTATCVLGRLSQELHRLQTYPRTNTGSNTY
SEQ ID NO: 101
KCNTATCVLG(Cit)LSQELHRLQTYPRTNTGSNTY
SEQ ID NO: 102
KCNTATCVLGRLSQELHRLQTYPRTNTGSNTY(4ABU)
SEQ ID NO: 103
Isocaproyl-KCNTATCVLGRLSQELHRLQTYPRTNTGSNTY(4ABU)
SEQ ID NO: 104
KCNTSTCATQRLANELVRLQTYPRTNVGSEAF
SEQ ID NO: 105
KCNTATCVLGRLSQELHRLQTYPTNVGSEAF
SEQ ID NO: 106
KCNTATCVLGRLSRSLHRLQTYPRTNTGSNTY
SEQ ID NO: 107
KCNTATCVTHRLSQELHRLQTYPRTNTGSNTY
SEQ ID NO: 108
KCNTATCVLGRLADFLHRLQTYPRTNTGSNTY
SEQ ID NO: 109
CNTATCVLGRLSQELHRLQTYPRTNTGSNT
SEQ ID NO: 110
KCNTATCVLGRLSQELHRLQNFVPRTNTGSNTY
SEQ ID NO: 111
KCNTATCVLGRLSQELHRLQTYPRTNTGSETF
SEQ ID NO: 112
ACDTATCVLGRLSQELHRLQTYPRTNTGSNTY
SEQ ID NO: 113
KCNTATCVLGRLSQELHRLQTYPRTNTGSKAF
SEQ ID NO: 114
KCDTATCVTHRLAGLLSRSQTYPRTNTGSNTY
SEQ ID NO: 115
KCNTATCVLGRLADALHRLQTYPRTNTGSNTY
SEQ ID NO: 116
KCNTATCVLGRLAAFLHRLQTYPRTNTGSNTY
SEQ ID NO: 117
SCNTATCVLGRLADFLHRLQTYPRTNTGSNTY
SEQ ID NO: 118
KCNTATCVLGRLSQELHRLQTMPRTNTGSNTY
SEQ ID NO: 119
KCNTATCVLGRLSQELHRLQTVPRTNTGSNTY
SEQ ID NO: 120
KCNTATCVLGRLNEYLHRLQTYPRTNTGSNTY
SEQ ID NO: 121
SCNTATCVLGRLSQELHRLQTYPRTNTGSNTY
SEQ ID NO: 122
KCNTATCVLGRLTEFLHRLQTYPRTNTGSNTY
SEQ ID NO: 123
KCNTATCVLGRLAEFLHRLQTYPRTNTGSNTY
SEQ ID NO: 124
KCNTATCVLGRLTDYLHRLQTYPRTNTGSNTY
SEQ ID NO: 125
KCNTATCVLGRLAQFLHRLQTYPRTNTGSNTY
SEQ ID NO: 126
KCNTATCVLGRLADFLHRFQTFPRTNTGSNTY
SEQ ID NO: 127
KCNTATCVLGRLADFLHRFHTFPRTNTGSNTY
SEQ ID NO: 128
KCNTATCVLGRLADFLHRFQTFPRTNTGSGTP
SEQ ID NO: 129
CNTATCVLGRLADFLHRLQTYPRTNTGSNTY
SEQ ID NO: 130
KCDTATCVLGRLSQELHRLQTYPRTNTGSNTY
SEQ ID NO: 131
KCNTATCVLGRLFDFLHRLQTYPRTNTGSNTY
SEQ ID NO: 132
KCNTATCVLGRLAAALHRLQTYPRTNTGSNTY
SEQ ID NO: 133
TCDTATCVLGRLSQELHRLQTYPRTNTGSNTY
SEQ ID NO: 134
CSNLSTCATQRLANELVRLQTYPRTNVGSNTY
SEQ ID NO: 135
KCNTATCATQRLANELVRLQTYPRTNVGSNTY
SEQ ID NO: 136
CSNLSTCVLGRLSQELHRLQTYPRTNTGSNTY
SEQ ID NO: 137
KCNTATCVLGRLSQELHRLQTYPRTNTGSNTY

With respect to the amino acid sequences described herein, Ac is acetyl; Agy is (S)-2-Amino-2-methyl-pent-4-enoic acid; Aib is alpha-methylalanine; 4Abu is 4-aminobutyric acid; dAh is (R)-2-amino-3-hydroxy-3-methyl-butyric acid; Ahp is (S)-2-amino-3-hydroxy-4-methyl-pentanoic acid; Ahb is (S)-2-amino-3-hydroxy-3-methyl-butyric acid; Hse is homoserine; Cit is citrulline; Orn is ornithine; 9Anc is H₂N(CH₂)₈CONH₂; and PEG5000 is polyethylene glycol having a molecular weight of about 5,000.

In other embodiments, the amylin agonist compounds useful in the methods described herein comprise at least a loop region, an α helix region, and a C-terminal tail. The loop region comprises an amino sequence comprising the formula X-(Xaa₁ sequence)-Y, where X and Y are capable of forming a bond and are independently selected residues having side chains which are chemically bonded to each other to form an intramolecular linkage such as disulfide bonds; amide bond; alkyl acids and alkyl amines which may form cyclic lactams; alkyl aldehydes or alkyl halides and alkylamines which may condensed and be reduced to form an alkyl amine or imine bridge; or side chains which may be connected to form an alkyl, alkenyl, alkynyl, ether or thioether bond. Alkyl chains may include lower alkyl groups having from about 1 to about 6 carbon atoms. In certain embodiments, the intramolecular linkage may be a disulfide, amide, imine, amine, alkyl and alkene bond. In certain embodiments, X and Y are independently selected from Ser, Asp, Glu, Lys, Orn, or Cys. In certain embodiments, X and Y are Cys and Cys. In other embodiments, X and Y are Ser and Ser. In still other embodiments, X and Y are Asp and Lys or Lys and Asp.

The Xaa₁ sequence comprises an amino acid sequence of 3, 4, 5, or 6 amino acids between X and Y. In certain embodiments, the Xaa₁ sequence comprises an amino acid sequence having a region with one or more substituted or unsubstituted hydroxyl-containing residues next to Y. For example, the hydroxyl containing residue region may have at least 2 of the 3 amino acids adjacent Y that are either Ser or Thr. The other amino acids in the Xaa₁ sequence may be any amino acid. In certain embodiments, the Xaa₁ sequence is 3 amino acids. In other embodiments, the Xaa₁ sequence is 4 amino acids. In still other embodiments, the Xaa₁ sequence is 5 amino acids. In yet other embodiments, the Xaa₁ sequence is 6 amino acids. Accordingly, Xaa₁ can be represented by Xaa₂Xaa₃Xaa₄Xaa₅Xaa₆Xaa₇. In certain embodiments, Xaa₂, Xaa₃, and/or Xaa₄ may absent. In certain embodiments, Xaa₅, Xaa₆, and Xaa₇ comprise the hydroxy-containing residue region. As such, at least two of the three amino acids can be a Ser, hSer, Thr, alloThr, d-Thr, or other unnatural analogs thereof. Xaa₂ can be any amino acid or absent, Xaa₃ can be any amino acid or absent, Xaa₄ can be any amino acid or absent, Xaa₅ can be any amino acid if Xaa₆ is a Ser or Thr and Xaa₇ is a Ser or Thr, Xaa₆ can be any amino acid if Xaa₅ is a Ser or Thr and Xaa₇ is a Ser or Thr, Xaa₇ can be any amino acid if Xaa₅ is Ser or Thr and Xaa₆ is Ser or Thr. Accordingly, in certain embodiment, Xaa₁ can be represented as Xaa₂ absent, Xaa₃ is Ala, Gly, Ser, Asp or absent, Xaa₄ is Asn, Ala, Asp, Gly or absent; Xaa₅ is Ala, Leu, Thr, or Ser; Xaa₆ is Ala, Ser, or Thr; and Xaa₇ is Ala, Ser, Val, Hse, (S)-2-amio-3-hydroxy-methylbutanoic acid (Ahb), (2S,3R)-2-amino-3hydroxy-methylpentanoic acid (Ahp), D-Thr, Thr, or a derivative thereof. In other embodiments Xaa₁ can be represented as Xaa₂ is absent, Xaa₃ is Ser, Gly, or absent, Xaa₄ is Asn or Asp, Xaa₅ is Ala, Ser, Thr or Leu, Xaa₆ is Ala, Thr or Ser, and Xaa₇ is Ser, D-Thr, alloThr or Thr. In certain embodiments, the loop region comprises the above-described representations of Xaa₁ wherein Xaa₃ is Ala, wherein Xaa₃ is Ser or wherein Xaa₃ is Gly. Alternatively or additionally, the loop region comprises the above described representations of Xaa₁ wherein Xaa₄ is Ala, wherein Xaa₄ is Asn, wherein Xaa₄ is Asp, or wherein Xaa₄ is Gly. Alternatively or additionally, the loop region comprises the above-described representations of Xaa₁ wherein Xaa₅ is Ala, wherein Xaa₅ is Thr, or wherein Xaa₅ is Leu. Alternatively or additionally, the loop region comprises the above described representations of Xaa₁ wherein Xaa₆ is Ser or wherein Xaa₆ is Ala. Alternatively or additionally, the loop region comprises the above-described representations of Xaa₁ wherein Xaa₇ is Thr or wherein Xaa₇ is D-Thr. It is further contemplated that no more than one, two, or three modifications such as substitutions, insertions, deletions, and/or derivatizations may be made to the loop region.

Examples of the loop region include, but are not limited to, C-N-T-A-T-C; C-A-T-A-T-C; C-D-T-A-T-C; C-G-T-A-T-C; C-N-A-A-T-C; C-N-T-S-T-C; C-N-T-A-dThr-C; C-N-T-A-T(OPO₃H₂)-C; C-N-T-A-S-C; C-N-T-A-A-C; C-N-T-A-V-C; C-N-T-A-Hse-C; C-N-T-A-Ahb-C; C-N-T-A-Ahp-C; C-S-N-L-S-T-C; C-G-N-L-S-T-C; C-A-N-L-S-T-C; C-S-A-L-S-T-C; C-S-N-A-S-T-C; C-S-N-L-A-T-C; and C-S-N-L-S-A-C. As previously noted, it is further contemplated that no more than one, two, or three modifications such as substitutions, insertions, deletions, and/or derivatizations may be made to the loop region.

The loop region may further comprise modifications or additional amino acids at the N-terminal end. Such modifications include the addition of compounds such as Lys, Ala, Phe, Ile, Ser, Octylglycine, Isocap, Fmoc-3,6-dioxyoctanoic acid, Fmoc-1-amino-4,7,10-trioxa-13-tridecanamine succinimic acid, acetyl, and/or groups for solubility, delivery, signaling. Exemplary modified loops include the addition of Lys the sequence of Xaa₁ or the addition of Ile to the sequence of Xaa₁. For example, the modified loop region may be K-C-N-T-A-T-C. In certain embodiments, the additions and/or modifications at the N-terminal end of the loop region may change the loop region. For example, the loop region may be modified as follows: cyclo(2,7) 1-7 hAmylin, cyclo(Asp²Lys⁷) 1-7 hAmylin, N-isocaproyl 1-7 hAmylin, N-3,6 dioxaoctanoyl 1-7 hAmylin, L-Octylglycine 1-7 hAmylin, Acetyl (Agy²,Agy⁷) 1-7 hAmylin wherein Agy is Allylglycine, Acetyl (Ala¹) 1-7 hAmylin, (Thr¹,Asp³) 1-7 hAmylin, Isocap (Ala⁷) 5-7 sCT, Acetyl (Agy²,Agy⁷) 1-7 sCT, and cyclo (1,7) (Asp¹,Lys⁷) 1-7 sCT the example of Isocap(7Ala) 5-7 sCT, certain embodiments comprise a modification at the N-terminal region of the loop region such that amino acids Xaa₂ to Xaa₅ are absent.

Throughout the application, the amino acid sequences may be referred to as amino acids at position a to position b adjacent to a reference peptide. In the present application the reference peptides are human amylin (hAmylin) (SEQ ID NO:1); rat amylin (rAmylin) (SEQ ID NO:15); salmon calcitonin (sCT) CSNLSTCVLGKLSQELHKLQTYPRTNTGSGTP (SEQ ID NO:141); and human calcitonin (hCT) CGNLSTCMLGTYTQDFNKFHTFPQTAIGVGAP (SEQ ID NO:140). For example in the previous paragraph, 1-7 hAmylin refers to the amino acid sequence from position 1 to position 7, inclusive, of human amylin (SEQ ID NO:1). Modification to the reference peptide may be shown as: position of modification adjacent to the modification. For example, (Asp²,Lys⁷) 1-7 hAmylin represents the amino acid sequence at positions 1 to 7 of human amylin with a modification of a Cys to Asp at position 2 and a modification of a Cys to Lys at position 7.

The α helix region of the compound may be about 8 to 23 amino acids in length. In certain embodiments, the α helix region is amphiphatic. In certain embodiments, the α helix region comprises about 3 to 6 helical turns. In certain embodiments, the α helix region comprises 3, 4, 5, or 6 helical turns. In other embodiments, the α helix region is a rigid structure equivalent to about 3, 4, 5, or 6 helical turns. An example of an idealized helix is LLQQLQKLLQKLKQY. In certain embodiments, the α helix is an ampliphatic structure. Accordingly, characteristics of desirable amino acids that would provide this type of structure may be selected.

It has been found that the calcitonin α helix region, a combination of an amylin and a calcitonin α helix region, or parts thereof, and/or some CGRP elements are desirable in the α helix region of the peptides. It is contemplated that, as with the loop region, the α helix region can be from any amylin or calcitonin, and analogs thereof. Accordingly, in certain embodiments, the α helix region is at least a portion of an α helix region of calcitonin or calcitonin analog. In other embodiments, the α helix region is at least a portion of an α helix region of calcitonin or calcitonin analog and at least a portion of an α helix of an amylin or amylin analog. In still other embodiments, the α helix region of the novel compounds contain elements of CGRP. It is further contemplated that novel compounds may have no more than one, two, three, four, or five further modifications such as substitutions, insertions, deletions, and/or derivatizations.

In certain embodiments, the α helix region may comprise amino acids from position 8 of sCT to position 18, 19, 20, 21, 22, 23, 24, 25, 26, or 27 of sCT. Moreover, the α helix region may comprise more than one portion of a calcitonin or calcitonin analog α helix region of the same or different species, for example 8-21 sCT 19-27 sCT; 8-21 sCT 18-27 sCT; or 8-16 hCT 17-27 sCT; or (Arg¹¹) 8-16 hCT (Arg¹⁸) 17-27 sCT. Alternatively or additionally, the above described α helix of 8-18 sCT to 8-27 sCT may further comprise the substitutions of one or more of (Aib¹⁰), (Arg¹¹), (Orn¹¹), (hArg¹¹), (Cit¹¹), (hLys¹¹), (Lys(for)¹¹), (Aib¹⁷), (Arg¹⁸), (Orn¹⁸), (hArg¹⁸), (Cit¹⁸), (hLys¹⁸), (Lys(for)¹⁸), (Lys(PEG5000)¹⁸), (Leu²²), (Pro²⁴) or any combination thereof.

In one embodiment, an α helix region can be represented by (α helix region type I) R₁-Val Leu Xaa₁₀Xaa₁₁Leu Ser Gln Xaa₁₅Leu Xaa₁Xaa₁₈ Leu Gln Thr Xaa₂₂ Pro Xaa₂₄ Thr Asn Thr-R₁, wherein Xaa₁₀ is Gly or Aib; Xaa₁₁ is Lys, Arg, Orn, hArg, Cit, hLys, or Lys(for); Xaa₁₅ is Glu or Phe; Xaa₁₇ is His or Aib; Xaa₁₈ is Lys, Arg, Orn, hArg, Cit, hLys,Lys(for), Lys(PEG 5000); Xaa₂₂ is Try or Leu; Xaa₂₄ is Arg or Pro; or R₁ is absent or comprises 1-4 additional amino acids.

Examples of an α helix region type I include 8-18 sCT, 8-21 sCT, 8-24 sCT, 8-27 sCT, (Arg¹¹) 8-18 sCT, (Arg¹⁸) 8-18 sCT, (Arg¹¹ Arg¹⁸) 8-18 sCT, (11Orn 18Orn) 8-18 sCT, (Arg¹¹ 18Cit) 8-18 sCT, (11hArg 18hArg) 8-18 sCT, (Arg¹¹ Orn¹⁸) 8-18 sCT, (11Cit Arg¹⁸) 8-18 sCT, (11Cit 18Cit) 8-18 sCT, (11hLys 18hLys) 8-18 sCT, (10Aib 11Arg 17Aib Arg¹⁸) 8-18 sCT, (11Lys(for) 18Lys(for)) 8-18 sCT, (10Aib 11Lys(for) 17Aib 18Lys(for)) 8-18 sCT, (Arg¹¹ 18Lys(PEG 5000)) 8-18 sCT, (Arg¹¹) 8-21 sCT, (Arg¹⁸) 8-21 sCT, (Arg¹¹ 18Arg) 8-21 sCT, (11Orn 18Orn) 8-21 sCT, (Arg¹¹ 18Cit) 8-21 sCT, (11hArg 18hArg) 8-21 sCT, (Arg¹¹ 18Orn) 8-21 sCT, (11Cit Arg¹⁸) 8-21 sCT, (11Cit 18Cit) 8-21 sCT, (11hLys 18hLys) 8-21 sCT, (10Aib Arg¹¹ 17Aib Arg¹⁸) 8-21 sCT, (11Lys(for) 18Lys(for)) 8-21 sCT, (10Aib 11Lys(for) 17Aib 18Lys(for)) 8-21 sCT, (Arg¹¹ 18Lys(PEG 5000)) 8-21 sCT, (Arg¹¹) 8-24 sCT, (Arg¹⁸) 8-24 sCT, (Arg¹¹ Arg¹⁸) 8-24 sCT, (Arg¹¹ Arg¹⁸ 22Leu) 8-24 sCT, (Arg¹¹ Arg¹⁸ 24Pro) 8-24 sCT, (11Orn 18Orn) 8-24 sCT, (Arg¹¹ 8Cit) 8-24 sCT, (11hArg 18hArg) 8-24 sCT, (Arg¹¹ 18Orn) 8-24 sCT, (11Cit Arg¹⁸) 8-24 sCT, (11Cit 18Cit) 8-24 sCT, (11hLys 18hLys) 8-24 sCT, (10Aib 11Arg 17Aib Arg¹⁸) 8-24 sCT, (11Lys(for) 18Lys(for)) 8-24 sCT, (10Aib 11Lys(for) 17Aib 18Lys(for)) 8-24 sCT, (Arg¹¹ 8Lys(PEG 5000)) 8-24 sCT, (Arg¹⁸) 8-27 sCT, (Arg¹⁸) 8-27 sCT, (Arg¹¹ Arg¹⁸) 8-27 sCT, (Arg¹¹ Arg¹⁸ 22Leu) 8-27 sCT, (Arg¹¹ Arg¹⁸ 24Pro) 8-27 sCT, (11Orn 18Orn) 8-27 sCT, (Arg¹¹ 18Cit) 8-27 sCT, (11hArg 18hArg) 8-27 sCT, (Arg¹¹ 180rn) 8-27 sCT, (11Cit Arg¹⁸) 8-27 sCT, (11Cit 18Cit) 8-27 sCT, (11hLys 18hLys) 8-27 sCT, (10Aib Arg¹¹ 17Aib Arg¹⁸) 8-27 sCT, (11Lys(for) 18Lys(for)) 8-27 sCT, (10Aib 11Lys(for) 17Aib 18Lys(for)) 8-27 sCT, (Arg¹¹ 8Lys(PEG 5000)) 8-27 sCT, (Arg¹¹ Arg¹⁸) 8-21 sCT-19-27 sCT, and (Arg¹¹ Arg¹⁸) 8-21 sCT-(18Leu) 18-27 sCT.

In certain embodiments, the α helix region may comprise a portion of an α helix region of amylin or amylin analog and a portion of an α helix region of calcitonin or calcitonin analog. The α helix region may comprise amino acids from position 8 of hAmylin to 11, 12, 13, 14, 15, 16, 17, 18 or 19 of hAmylin and amino acids from position 13, 14, 15, 16, 17, 18, and 19 of sCT to position 18, 19, 20, 21, 22, 23, 24, 25, 26, or 27 of sCT. Alternatively or additionally, the above described α helix region of amylin and calcitonin may further comprise the substitutions of one or more of (8Val), (9Leu), (9Met), (10Gly), (10His), (12Thr), (13Thr), (13Asn), (13Phe), (13Tyr), (14Arg), (14Ala), (14Asp), (14Glu), (14Gln), (14Thr), (14Gly), (15Leu), (15Ser), (15Glu), (15Ala), (15Tyr), (16Asp), (17Ser), (17Phe), (17Arg), (17Aib), (Arg¹⁸), (18Orn), (18hArg), (18Cit), (18hLys), (18Lys(for)), (18Lys(PEG5000)), (19Phe), (20His), (21Asn), (22Met), (22Val), (22Phe), (22Leu), (24Pro), or any combination thereof. In certain embodiments, the number of amino acids in the α helix region is at least 10 amino acids. In other embodiments, the number of amino acids in the α helix region is 11, 12, 13, 14, 15, 16, 17, 18, 19, 20, 21, 22, or 23. In other embodiments, the number of amino acids in the α helix region is 24 or more.

In one embodiment, an α helix region can be represented by (α helix region type II) R₁-Xaa₈ Xaa₉ Xaa₁₀ R Xaa₁₂ Xaa₁₃ Xaa₁₄ Xaa₁₅ Xaa₁₆ Xaa₁₇ Xaa₁₈ Xaa₁₉ Xaa₂₀ Xaa₂₁ Xaa₂₂ P Xaa₂₄ TNT-R₁ wherein Xaa₈ is Ala or Val; Xaa₉ is Thr, Met or Leu; Xaa₁₀ is Gln, Gly, His; Xaa₁₂ is Leu, or Thr; Xaa₁₃ is Ala, Thr, Asn, Phe, Tyr, Ser, or Thr; Xaa₁₄ is Asn, Arg, Ala, Asp, Glu, Gln, Thr, or Gly; Xaa₁₅ is Phe, Leu, Ser, Glu, Ala, Asp, or Tyr; Xaa₁₆ is Leu or Asp; Xaa₁₇ is Val, His, Ser, Phe, or Aib; Xaa₁₈ is His, Arg, Lys, Orn, hArg, Cit, hLys, Lys(for), or Lys(PEG5000); Xaa₁₉ is Leu, Ser or Phe; Xaa₂₀ is Gln or His; Xaa₂₁ is Thr or Asn; Xaa₂₂ is Tyr, Val, Phe,Leu or Met; Xaa₂₄ is Arg or Pro; and R₁ is absent or comprises 1-4 additional amino acids.

Examples of an α helix region of type II include, but is not limited to (8Val 9Leu 10Gly) 11-15 hAmylin 16-27 sCT, (8Val 9Leu 10Gly) 11-15 hAmylin (18Arg) 16-27 sCT, 8-12 hAmylin (18Arg) 13-27 sCT, 8-18 hAmylin 19-23 sCT, 8-18 HAmylin 19-27 sCT, (15Glu 18Arg) 8-18 hAmylin 19-24 sCT, (14Arg 15Ser) 8-18 hAmylin 19-22 sCT, (13Ala 14Ala 15Ala 8-18 hAmylin 19-27 sCT, (13Ala 14Asp 15Ala) 8-18 hAmylin 19-22 sCT, (13Ala 14Asp) 8-18 hAmylin 19-23 sCT, (13Ala 14Asp) 8-18 hAmylin 19-27 sCT, (13Ala 14Ala) 8-18 hAmylin 19-22 sCT, (13Ala 14Glu) 8-18 hAmylin 19-22 sCT, (13Thr 14Asp 15Tyr) 8-18 hAmylin 19-22 sCT, (13Ala 14Gln) 8-18 hAmylin 19-22 sCT, (13Asn l4Glu l5Tyr) 8-18 hAmylin 19-27 sCT, (13Phe 14Asp) 8-18 hAmylin 19-27 sCT, (13Ala 14Asp) 8-18 hAmylin (15Glu 18Arg) 8-18 hAmylin 19-24 sCT, (19Phe 22Phe) 19-27 sCT, (13Ala l4Asp) 8-18 hAmylin (19Phe 20His 22Phe) 19-27 sCT, (13Ala 14Asp) 8-18 hAmylin (19Phe 22Phe) 19-27 sCT, (9Thr 10His) 8-18 hAmylin 19-22 sCT, (9Thr 10His 14Gly 15Leu 17Ser 18Arg) 8-19 hAmylin 20-23 sCT, 8-18 hAmylin (21Asn 22Phe 23Val) 19-23 sCT, 8-18 hAmylin (22Met) 19-27 sCT, 8-18 hAmylin (22Val) 19-27 sCT, (9Met 12Thr 13Tyr 14Thr 15Glu 16Asp 17Phe) 8-17 hAmylin (18Arg) 18-20 sCT). In other embodiments, novel compounds include variations of the above exemplary compounds with the α helix terminating at corresponding to 22, 23, 24, 25, 26 or 27 of sCT. In other words, compound 8-18 hAmylin 19-24 sCT is also specifically described as this compound is merely 8-18 hAmylin 19-27 sCT described above truncated to position 24. As another example, compound (13Ala l4Asp 15Ala) 8-18 hAmylin 19-23 is specifically described because of the above language applied to (13Ala l4Asp 15Ala) 8-18 hAmylin 19-22.

In certain embodiments, the C-terminal tail comprises amino acids from position 27, 28, 29, 30, 31, 32, or 33 to position 36 or 37 of hAmylin. In other embodiments, the C-terminal tail comprises amino acids from position 27 or 28 to position 32 of sCT; however, when the loop region is from a calcitonin or calcitonin analog and the α helix region is from a calcitonin or calcitonin analog, the last position of the C-terminal tail is not Pro, Hyp, homoSerine (Hse) or derivatives of Hse. Alternatively or additionally, the above described α helix of amylin and calcitonin may further comprise the substitutions of one or more of (27Tyr) hAmylin, (29Arg) hAmylin, (32Val) hAmylin, (32Thr) hAmylin, (34Glu) hAmylin, (35Lys) hAmylin, (36Phe) hAmylin, (36Ala) hAmylin, (37Phe) hAmylin, (30Asn) sCT, (32Tyr) sCT, or any combination thereof.

In one embodiment, a C-terminal tail can be represented by Xaa₂₈ Xaa₂₉ Xaa₃₀ Xaa₃₁ Xaa₃₂ Xaa₃₃ G Xaa₃₅ Xaa₃₆ Xaa₃₇ Xaa₃₈, wherein Xaa₂₈ is Lys, Tyr, or absent; Xaa₂₉ is Ser, Pro, or absent; Xaa₃₀ is Ser, Pro, Arg, or absent; Xaa₃₁ is Thr, or absent; Xaa₃₂ is Asn or absent; Xaa₃₃ is Val, Thr, or absent; Xaa₃₅ is Ser, Glu; Xaa₃₆ is Asn, Lys, or Gly; Xaa₃₇ is Thr, Phe, or Ala; Xaa₃₈ is Tyr, Phe, Pro, or absent; with the proviso that when the loop region is from a calcitonin or calcitonin analog and the α helix region is from a calcitonin or calcitonin analog, the last position of the C-terminal tail is not Pro, Hyp, homoSerine (Hse) or derivatives of Hse.

Examples of the C-terminal tail include, but is not limited to, 27-37 rAmylin, (27Tyr 29Arg 32Thr) 27-37 rAmylin, (29Arg 32Thr) 28-37 rAmylin, 30-37 hAmylin, (32Thr) 30-37 hAmylin, (35Lys 36Ala 37Phe) 30-37 hAmylin, 30-36 hAmylin, (32Val) 30-36 hAmylin, (34Glu 36Phe) 30-36 hAmylin, 31-37 hAmyin, 31-36 hAmylin, 33-36 hAmylin, 33-7 hAmylin, 28-32 sCT, (30Asn 32Tyr) 28-32 sCT, and 27-32 sCT. In other embodiments, the C-terminal tail comprises the amino acid sequence KSNFVPTN or SNFVPTNV.

It is further contemplated that no more than one, two, or three modifications such as substitutions, insertions, deletions, and/or derivatizations may be made to the C-terminal tail as described in the preceding paragraphs. The C-terminal tail of the novel compounds may further comprise modifications or additional amino acids at the C-terminal end. Such modifications include the addition of compounds such as Lys, up to 4 Lys, L-Octylglycine, 4ABU (4-Aminobutyric acid), 9Anc (9-Amiononanoic acid), and/or groups for solubility, stability, or delivery. Examples include 33-37 hAmylin L-octylglycine, 33-37 hAmylin 4ABU, and 33-37 hAmylin 9Anc.

In yet another aspect, the following peptides may be used in the methods described herein including peptides that comprise an amino acid sequence of Formula (VII): Xaal X Xaa3 Xaa4 Xaa5 Xaa6 Y Xaa8 Xaa9 Xaa10 Xaa11 Xaa12 Xaa13 Xaa14 Xaa15 Xaa16 Xaa17 Xaa18 Xaa19 Xaa20 Xaa21 Xaa22 Xaa23 Xaa24 Xaa25 Xaa26 Xaa27 Xaa28 Xaa29 Xaa30 Xaa31 Xaa32, wherein Xaa1 is A, C, hC, D, E, F, I, L, K, hK, R, hR, S, Hse(homoSER), T, G, Q, N, M, Y, W, P, Hyp(hydroxyProline), H, V or absent; Xaa3 is A, D, E, N, Q, G, V, R, K, hK, hR, H, I, L, M, or absent; Xaa4 is A, I, L, S, Hse, T, V, M, or absent; Xaa5 is A, S, T, Hse, Y, V, I, L, or M; Xaa6 is T, A, S, Hse, Y, V, I, L, or M; Xaa8 is A, V, I, L, F, or M; Xaa9 is L, T, S, Hse, V, I, or M; Xaa10 is G, H, Q, K, R, N, hK, or hR; Xaa11 is K, R, Q, N, hK, hR, or H; Xaa12 is L, I, V, F, M, W, or Y; Xaa13 is A, F, Y, N, Q, S, Hse, orT; Xaa14 is A, D, E, G, N, K, Q, R, H, hR, or hK; Xaa15 is A, D, E, F, L, S, Y, I, V, or M; Xaa16 is L, F, M, V, Y, or I; Xaa17 is H, Q, N, S, Hse, T, or V; Xaa18 is K, hK, R, hR, H, u (Cit), or n (Orn); Xaa19 is F, L, S, Hse,V, I, T, or absent; Xaa20 is H, R, K, hR, hK, N, Q, or absent; Xaa2l is T, S, Hse, V, I, L, Q, N, or absent; Xaa22 is F, L, M, V, Y, or I; Xaa23 is P or Hyp; Xaa24 is P, Hyp, R,K, hR, hK, or H; Xaa25 is T, S, Hse, V, I, L, F, or Y; Xaa26 is N,Q, D, or E; Xaa27 is T, V, S, F, I, or L; Xaa28 is G or A; Xaa29 is S, Hse, T, V, I, L, or Y; Xaa30 is E, G, K, N, D, R, hR, hK, H, or Q; Xaa31 is A, T, S, Hse, V, I, L, F, or Y; and Xaa32 is F, P, Y, Hse, S, T, or Hyp; wherein X and Y are capable of creating a bond and are independently selected residues having side chains which are chemically bonded to each other to form an intramolecular linkage such as disulfide bonds; amide bond; alkyl acids and alkyl amines which may form cyclic lactams; alkyl aldehydes or alkyl halides and alkylamines which may condensed and be reduced to form an alkyl amine or imine bridge; or side chains which may be connected to form an alkyl, alkenyl, alkynyl, ether or thioether bond. Alkyl chains may include lower alkyl groups having from about 1 to about 6 carbon atoms. In certain embodiments, the intramolecular linkage may be a disulfide, amide, imine, amine, alkyl and alkene bond. In certain embodiments, X and Y are independently selected from Ser, Asp, Glu, Lys, Orn, or Cys. In certain embodiments, X and Y are Cys and Cys. In other embodiments, X and Y are Ser and Ser. In still other embodiments, X and Y are Asp and Lys or Lys and Asp.

In yet another aspect, the peptides may comprise an amino acid sequence of Formula (VIII): Xaa1 Xaa2 Xaa3 Xaa4 Xaa5 Xaa6 Xaa7 Xaa8 Xaa9 Xaa10 Xaa11 Xaa12 Xaa13 Xaa14 Xaa15 Xaa16 Xaa17 Xaa18 Xaa19 Xaa20 Xaa21 Xaa22 Xaa23 Xaa24 Xaa25 Xaa26 Xaa28 Ser Xaa30 Xaa31 Xaa32, wherein Xaa1 is A, C, D, F, I, K, S, T, or absent; Xaa2 is C, D, S, or absent; Xaa3 is A, D, N, or absent; Xaa4 is A, L, T, or absent; Xaa5 is A or S; Xaa6 is T, A, S, or V; Xaa7 is C, K, or A; Xaa8 is A, V, L, or M; Xaa9 is L or T; Xaa10 is G, H, or Q; Xaa11 is K, R, Q, or hArg; Xaa12 is L, W, or Y; Xaa13 is A, F, N, Q, S, or T; Xaa14 is A, D, E, G, N, K, Q, or R; Xaa15 is A, D, E, F, L, S, or Y; Xaa16 is L, or F; Xaa17 is H, Q, S, or V; Xaa18 is K, R, hArg, u (Cit), or n (Orn); Xaa19 is F, L, S, or absent; Xaa20 is H, Q, or absent; Xaa21 is T, N, or absent; Xaa22 is F, L, M, V, or Y; Xaa23 is P; Xaa24 is P or R; Xaa25 is T; Xaa26 is N; Xaa27 is T or V; Xaa28 is G; Xaa30 is E, G, K, or N; Xaa31 is A or T; and Xaa32 is F, P, or Y.

In yet another aspect, compounds comprise an amino acid sequence of Formula (IX): Xaa1 Xaa2 Xaa3 Xaa4 Xaa5 Xaa6 Xaa7 Xaa8 Xaa9 Xaa10 Xaa11 Leu Xaa13 Xaa14 Xaa15 Leu Xaa17 Xaa18 Xaa19 Xaa20 Xaa21 Xaa22 Pro Xaa24 Thr Asn Xaa27 Gly Ser Xaa30 Xaa31 Xaa32, wherein Xaal is A, C, F, I, K, S, or absent; Xaa2 is C, D, or S; Xaa3 is A, D or N; Xaa4 is A, L or T; Xaa5 is A or S; Xaa6 is T; Xaa7 is C or K; Xaa8 is A or V; Xaa9 is L or T; Xaa10 is G, H, or Q; Xaa11 is K, R, or hArg; Xaa13 is A, F, N, S, or T; Xaa14 is A, D, E, G, N, Q, or R; Xaa15 is A, E, F, L, S, or Y; Xaa17 is H, S, or V; Xaa18 is K, R, hArg, u (Cit), or n (Orn); Xaa19 is F, L, or S; Xaa20 is H or Q; Xaa21 is T or N; Xaa22 is F, L, M, V, or Y; Xaa24 is P or R; Xaa27 is T, or V; Xaa30 is E, G, K, or N; Xaa31 is A, or T; and Xaa32 is F, P, or Y.

In a general aspect, the sequences of Formula (VII), (VIII), or (IX) further comprises 1, 2, 3, 4, 5 or more modifications of substitutions, insertions, deletions, elongations and/or derivatizations. In certain embodiments, the squence of formula I, II, or III comprises a Val is inserted between amino acids at positions 22 and 23. In other embodiments, the sequence of formula I, II, or II comprises a Gln is inserted between positions 22 and 23. In still other embodiments, the sequence of formula I, II, or III comprises a sequence of GlnThrTyr between positions 22 and 23. In yet other embodiments, the sequence of formula I, II, or III comprises a sequence of Leu-Gln-Thr-Tyr between positions 22 and 23. In another general aspect, the modifications of formula I, II, or III may be at the N-terminal end. In certain embodiments, the N-terminal portion of formula I, II, or III has an added octylglycine . In other embodiments, the N-terminal portion of formula I, II or III has an added isocap.

In yet another aspect, suitable peptides comprise an amino acid sequence of Formula (X): Xaa1 Xaa2 Xaa3 Xaa4 Xaa5 Xaa6 Xaa7 Xaa8 Xaa9 Xaa10 Xaa11 Xaa12 Xaa13 Xaa14 Xaa15 Xaa16 Xaa17 Xaa18 Xaa19 Xaa20 Xaa2l Xaa22 Pro Xaa24 Thr Asn Xaa27 Gly Ser Xaa30 Xaa31 Xaa32, wherein Xaa1 is A, C, D, F, K, T, or absent; Xaa2 is A, C, D, S, or absent; Xaa3 is A, D, N, or absent; Xaa4 is A, L, T, or absent; Xaa5 is A or S; Xaa6 is A, S, T, or V; Xaa7 is A, C, or K; Xaa8 is A, L, M, or V; Xaa9 is L or T; Xaa10 is G, H, or Q; Xaa11 is K, Q, or R; Xaa12 is L, W, or Y; Xaa13 is A, N, Q, S, or T; Xaa14 is A, D, E, G, K, N, Q, or R; Xaa15 is A, D, E, F, L, S, or Y; Xaa16 is F or L; Xaa17 is H, Q, S or V; Xaa18 is K, or R; Xaa19 is F, L, S, or absent; Xaa20 is H, K, Q, or absent; Xaa21 is Q, T, or absent; Xaa22 is F, L, or Y; Xaa24 is P or R; Xaa27 is T or V; Xaa30 is E, K or N; Xaa31 is A or T; Xaa32 is F,Y, or absent;

In yet another aspect, suitable peptides comprise an amino acid sequence comprising: (a) a loop region comprising Xaaᵢ; (b) an α helix loop type I; and (c) a C-terminal tail;

wherein Xᵢ comprises an amino sequence of X Xaa₂ Xaa₃ Xaa₄ Xaa₅ Xaa₆ Xaa₇ Y, wherein Xaa₂ is any amino acid or absent; Xaa₃ is Ala, Gly, Ser, Asp or absent; Xaa₄ is Asn, Ala, Asp, Gly or absent; Xaa₅ is Ala, Leu, Thr, or Ser; Xaa₆ is Ala, Ser, or Thr; and Xaa₇ is Ala, Ser, Val, Hse, (S)-2-amio-3-hydroxy-methylbutanoic acid (Ahb), (2S,3R)-2-amino-3hydroxy-methylpentanoic acid (Ahp), D-Thr, Thr, or a derivative thereof; X and Y are amino acids capable of creating a bond and are independently selected residues having side chains which are chemically bonded to each other to form an intramolecular linkage such as disulfide bonds; amide bond; alkyl acids and alkyl amines which may form cyclic lactams; alkyl aldehydes or alkyl halides and alkylamines which may condensed and be reduced to form an alkyl amine or imine bridge; or side chains which may be connected to form an alkyl, alkenyl, alkynyl, ether or thioether bond;

the a helical region type I comprises the sequence R₁-Val Leu Xaa₁₀ Xaa₁₁ Leu Ser Gln Xaa₁₅ Leu Xaa₁₇ Xaa₁₈ Leu Gln Thr Xaa₂₂ Pro Xaa₂₄ Thr Asn Thr-R₁, wherein Xaa₁₀ is Gly or Aib; Xaa₁₁ is Lys, Arg, Orn, hArg, Cit, hLys, or Lys(for); Xaa₁₅ is Glu or Phe; Xaa₁₇ is His or Aib; Xaa₁₈ is Lys, Arg, Orn, hArg, Cit, hLys,Lys(for), Lys(PEG 5000); Xaa₂₂ is Try or Leu; Xaa₂₄ is Arg or Pro; or R₁ is absent or comprises 1-4 additional amino acids; and the C-terminal tail comprises the sequence Xaa₂₈Xaa₂₉Xaa₃₀Xaa₃₁Xaa₃₂Xaa₃₃GlyXaa₃₅ Xaa₃₆Xaa₃₇Xaa₃₈, wherein Xaa₂₈ is Lys, Tyr, or absent; Xaa₂₉ is Ser, Pro, or absent; Xaa₃₀ is Ser, Pro, Arg, or absent; Xaa₃₁ is Thr, or absent; Xaa₃₂ is Asn or absent;Xaa₃₃ is Val, Thr, or absent; Xaa₃₅ is Ser or Glu; Xaa₃₆ is Asn, Lys, or Gly; Xaa₃₇ is Thr, Phe, or Ala; Xaa₃₈ is Tyr, Phe, Pro, or absent; with the proviso that when the loop region is from a calcitonin or calcitonin analog and the α helix region is from a calcitonin or calcitonin analog, the last position of the C-terminal tail is not Pro, Hyp, homoSerine (Hse) or derivatives of Hse.

In yet another aspect, suitable peptides comprise an amino acid sequence comprising (a) a loop region comprising Xaa₁; (b) an α helix loop type II; and (c) a C-terminal tail;

wherein loop region Xaa₁ comprises an amino sequence of X Xaa2 Xaa3 Xaa4 Xaa5 Xaa6 Xaa7 Y wherein, Xaa2 is any amino acid or absent; Xaa3 is Ala, Gly, Ser, Asp or absent; Xaa4 is Asn, Ala, Asp, Gly or absent; Xaa5 is Ala, Leu, Thr, or Ser; Xaa6 is Ala, Ser, or Thr; and Xaa7 is Ala, Ser, Val, Hse, (S)-2-amio-3-hydroxy-methylbutanoic acid (Ahb), (2S,3R)-2-amino-3hydroxy-methylpentanoic acid (Ahp), D-Thr, Thr, or a derivative thereof; X and Y are amino acids capable of creating a bond and are independently selected residues having side chains which are chemically bonded to each other to form an intramolecular linkage such as disulfide bonds; amide bond; alkyl acids and alkyl amines which may form cyclic lactams; alkyl aldehydes or alkyl halides and alkylamines which may condensed and be reduced to form an alkyl amine or imine bridge; or side chains which may be connected to form an alkyl, alkenyl, alkynyl, ether or thioether bond;

the α helical region type II comprises the sequence R1-Xaa8 Xaa9 Xaa10 R Xaa12 Xaa13 Xaa14 Xaa15 Xaa16 Xaa17 Xaa18 Xaa19 Xaa20 Xaa2l Xaa22 P Xaa24 TNT-R1, wherein Xaa8 is Ala or Val; Xaa9 is Thr, Met or Leu; Xaa10 is Gln, Gly, His; Xaa12 is Leu, or Thr; Xaa13 is Ala, Thr, Asn, Phe, Tyr, Ser, or Thr; Xaa14 is Asn, Arg, Ala, Asp, Glu, Gln, Thr, or Gly; Xaa15 is Phe, Leu, Ser, Glu, Ala, Asp, or Tyr; Xaa16 is Leu or Asp; Xaa17 is Val, His, Ser, Phe, or Aib; Xaa18 is His, Arg, Lys, Orn, hArg, Cit, hLys, Lys(for), or Lys(PEG5000); Xaa19 is Leu, Ser or Phe; Xaa20 is Gln or His; Xaa21 is Thr or Asn; Xaa22 is Tyr, Val, Phe,Leu or Met; Xaa24 is Arg or Pro; and R1 is absent or comprises 1-4 additional amino acids; and the C-terminal tail comprises the sequence Xaa28 Xaa29 Xaa30 Xaa31 Xaa32 Xaa33 G Xaa35 Xaa36 Xaa37 Xaa38, wherein Xaa28 is Lys, Tyr, or absent; Xaa29 is Ser, Pro, or absent; Xaa30 is Ser, Pro, Arg, or absent; Xaa31 is Thr, or absent; Xaa32 is Asn or absent; Xaa33 is Val, Thr, or absent; Xaa35 is Ser or Glu; Xaa36 is Asn, Lys, or Gly; Xaa37 is Thr, Phe, or Ala; and Xaa38 is Tyr, Phe, Pro, or absent.

The compounds described herein can form pharmaceutically acceptable salts with various inorganic acids, organic acids, and bases. Exemplary salts prepared with organic and inorganic acids include HCl, HBr, H₂SO₄, H₃PO₄, trifluoroacetic acid, acetic acid, formic acid, methanesulfonic acid, toluenesulfonic acid, maleic acid, fumaric acid, camphorsulfonic acid, and the like. Exemplary salts prepared with bases include ammonium salts, alkali metal salts (such as sodium and potassium salts) and alkali earth salts (such as calcium and magnesium salts). In one embodiment, the pharmaceutically acceptable salt is an acetate salt, a hydrochloride salt, or a trifluoroacetate salt. The pharmaceutically acceptable salts may be formed by conventional means, as by reacting the free acid or base forms of the compounds with one or more equivalents of the appropriate base or acid in a solvent or medium in which the salt is insoluble, or in a solvent such as water, which is then removed in vacuo or by freeze-drying, or by exchanging the ions of an existing salt for another ion on a suitable ion exchange resin.

The peptides described herein may be prepared using conventional coupling reactions known in the art. For example, the peptides may be prepared by successively adding the desired amino acid to a growing peptide chain. Typically, an alpha-N-carbamoyl protected amino acid and an amino acid attached to the growing peptide chain on a resin support are reacted at room temperature in an inert solvent such as N-methylpyrrolidone, dimethylformamide or methylene chloride in the presence of coupling agents such as dicyclohexylcarbodiimide 1-hydroxybenzotriazole in the presence of a base such as diisopropylethylamine. The alpha-N-carbamoyl protecting group is removed from the resultant peptide with a reagent such as trifluoroacetic acid or piperidine, and the coupling reaction repeated with the next desired N-protected amino acid. Suitable N-protecting groups are known in the art, with t-butyloxycarbonyl herein preferred.

Suitable coupling conditions include use of a solvent system which maximizes swelling of the solid support, minimizes secondary structure elements of the peptide chain during synthesis cycles, and minimizes intrapeptide and interpeptide hydrogen bonding. Preferably the synthesis cycle includes a capping step after the coupling step(s) wherein unreacted alpha-amino groups of the peptide chain are rendered unreactive. The synthesis cycle is successively repeated using appropriate protected alpha-amino acids to give amylin or an amylin analog of specified sequence. After completion of the successive synthesis cycles, the peptides are cleaved from the solid support. It is preferred that the cysteine residues of the peptide chain are selectively deprotected and an intramolecular disulfide bond is formed before cleaving the peptide bond from the solid support.

Peptides may be purified by RP-HPLC (preparative and analytical) using a Waters Delta Prep 3000 system. A C4, C8 or C18 preparative column (10μ, 2.2×25 cm; Vydac, Hesperia, Calif.) may be used to isolate peptides, and purity may be determined using a C4, C8 or C18 analytical column (5μ, 0.46×25 cm; Vydac). Solvents (A=0.1% TFA/water and B=0.1% TFA/CH₃ CN) may be delivered to the analytical column at a flowrate of 1.0 ml/min and to the preparative column at 15 ml/min. Amino acid analyses may be performed on the Waters Pico Tag system and processed using the Maxima program. Peptides may be hydrolyzed by vapor-phase acid hydrolysis (115° C., 20-24 h). Hydrolysates may be derivatized and analyzed by standard methods (Cohen, et al., The Pico Tag Method: A Manual of Advanced Techniques for Amino Acid Analysis, pp. 11-52, Millipore Corporation, Milford, Mass. (1989)). Fast atom bombardment analysis may be carried out by M-Scan, Incorporated (West Chester, Pa.). Mass calibration may be performed using cesium iodide or cesium iodide/glycerol. Plasma desorption ionization analysis using time of flight detection may be carried out on an Applied Biosystems Bio-Ion 20 mass spectrometer.

The compounds described herein may also be prepared following U.S. Pat. No. 5,686,411, U.S. Pat. No. 5,424,394, and US Publication No. 2008/0274952, the disclosures of which are incorporated by reference herein. These methods provide for the solid phase synthesis of amylin agonist peptides using successive synthesis cycles, whereby in each synthesis cycle, a designated amino acid is added to a growing peptide chain attached to an insoluble resin support by formation of a peptide linkage between an α-amino group of the growing peptide chain and on α-carboxyl of the designated amino acid; and wherein each synthesis cycle comprises: (i) treating the growing peptide chain under α-amino deprotecting conditions to remove an α-amino group; (ii) activating the α-carboxyl group of the α-amino protected designated amino acid; (iii) contacting the growing peptide chain and the designated amino acid under coupling conditions to form a peptide linkage between the free α-amino for the peptide chain and the activated α-carboxyl of the designated amino acid; and (iv) repeating steps (ii) and (iii) as necessary.

The peptides described herein may also be prepared using recombinant DNA techniques, using methods now known in the art, such as those described in Sambrook et al., Molecular Cloning: A Laboratory Manual, 2d Ed., Cold Spring Harbor (1989).

The compounds described herein can be linked to one or more polymers to provide additional beneficial biological properties. Such additional beneficial biological properties may include, e.g., providing additional therapeutic activity to the compound; increasing the in vivo half life of the compound, decreasing the rate of clearance of the compound by the kidney, decreasing the immunogenicity of the compound, decreasing the proteolysis rate of the compound, or increasing the stability of the compound. Exemplary polymers that can be linked to the amylin agonist compounds include peptides, saccharides, polyethylene glycols, albumin, fatty acids, polyamino acids, dextran, gelatin, polyvinyl pyrrolidone, polyvinyl alcohol, N-(2-hydroxypropyl)-methacrylamide, and the like. In one embodiment, the amylin agonist compounds are linked to peptides, saccharides, polyethylene glycols, albumin, fatty acids, and polyamino acids.

In one embodiment, the amylin agonist compounds described herein are linked to peptides that have different therapeutic activity and/or that target different receptors than the amylin agonist compound. Exemplary peptides include GLP-1 receptor agonists (e.g., exendins, exendin analogs, GLP-1(7-37), GLP-1(7-37) analogs); PYY; PYY analogs; leptin; leptin analogs; GIP; GIP analogs, and the like. Amylin agonist compounds linked (e.g., directly or through amino acid(s) and/or chemical moiety linkers) to another peptide may be referred to as hybrid peptides. Examples of hybrid peptides comprising amylin agonist peptides linked to other therapeutic peptides are described, for example, in WO 2005/077072 and WO 2007/022123, the disclosures of which are incorporated by reference herein.

In one embodiment, the compounds described herein are linked to saccharides (e.g., N-acetyl-galactosamine, N-acetyl-glucosamine, galactose, sialic acid, glucose, fucose, mannose, and the like) to produce glycans. Such compounds may be referred to as glycosylated peptides. The amylin agonist peptides can be glycosylated (e.g., N-linked glycosylation, O-linked glycosylation) at, e.g., an Asn amino acid residue, a Ser amino acid residue, a Thr amino acid residue, or any combination of two or more thereof. Methods for the glycosylation of amino acids are known in the art and described, for example, in U.S. Pat. No. 5,854,391, the disclosure of which is incorporated herein by reference.

In one embodiment, the compounds described herein are linked to one or two polyethylene glycols, preferably one polyethylene glycol. The polyethylene glycol can have a molecular weight from about 5,000 daltons to about 40,000 daltons. In one embodiment, the compounds described herein are linked to a polyamino acid. Exemplary polyamino acids include poly-lysine (e.g., poly-D-lysine and/or poly-L-lysine), poly-aspartic acid, poly-serine, poly-glutamic acid, and the like. In one embodiment, the compounds described herein are linked to a fatty acid. The fatty acid may be a C₄-C₂₈ fatty acid chain that is saturated or unsaturated, and branched or linear.

When the compounds described herein are linked to one or more polymers, any linking group known in the art can be used. The linking group may comprise any chemical group(s) suitable for linking the compound to the polymer. Exemplary linking groups include amino acids, maleimido groups, dicarboxylic acid groups, succinimide groups, or a combination of two or more thereof Alternatively, the compound can be directly attached to the polymer without any linking group. Methods for linking compounds to one or more polymers are known in the art and described, for example, in U.S. Pat. No. 6,329,336; U.S. Pat. No. 6,423,685; U.S. Pat. No. No. 6,924,264; WO 2007/022123; WO 2007/053946; WO 2008/058461; and WO 2008/082274, the disclosures of which are incorporated by reference herein.

In other embodiment, the compounds may have other chemical modification that may involve adding chemical moieties, creating new bonds, and removing chemical moieties. Examplary modifications at amino acid side groups include acylation of lysine ε-amino groups, N-alkylation of arginine, histidine, or lysine, alkylation of glutamic or aspartic carboxylic acid groups, and deamidation of glutamine or asparagine. Exemplary modifications of the terminal amino group include the desamino, N-lower alkyl, N-di-lower alkyl, and N-acyl modifications, such as alkyl acyls, branched alkylacyls, alkylaryl-acyls. Exemplary modifications of the terminal carboxy group include the amide, lower alkyl amide, dialkyl amide, arylamide, alkylarylamide and lower alkyl ester modifications. Lower alkyl is C₁₋₄ alkyl. Furthermore, one or more side groups, or terminal groups, may be protected by protective groups known to the skilled artisan. The α-carbon of an amino acid may be mono- or dimethylated.

Pharmaceutical compositions containing the amylin agonist compounds described herein may be provided in the form of solutions suitable for peripheral administration, including parenteral (including intravenous, intraarterial, intramuscular, subcutaneous), nasal, or oral administration. Pharmaceutical compositions containing the amylin agonist compounds described herein can be prepared following the teachings in, for example, U.S. Pat. No. 5,998,367 and U.S. Pat. No. 6,410,511, the disclosures of which are incorporated by reference herein. Other references for preparing pharmaceutical compositions containing compounds can be considered, including, for example, Remington's Pharmaceutical Sciences by Martin; and Wang et al, Journal of Parenteral Science and Technology, Technical Report No. 10, Supp. 42:2S (1988), the disclosures of which are incorporated by reference herein.

The pharmaceutical formulations may be stabilized at neutral pH. Since the amylin agonist compounds are amphoteric they may be utilized as free bases, as acid addition salts, or as metal salts. A wide variety of pharmaceutically acceptable acid addition salts are available, as described above. These include those prepared from both organic and inorganic acids, preferably mineral acids. Typical acids which may be mentioned by way of example include acetic, citric, succinic, lactic, hydrochloric and hydrobromic acids. Such products are readily prepared by procedures well known in the art.

The amylin agonist compounds will normally be provided as parenteral compositions for injection or infusion. They can, for example, be suspended in an inert oil, suitably a vegetable oil such as sesame, peanut, or olive oil. Alternatively, they can be suspended in an aqueous isotonic buffer solution at a pH of about 5.6 to 7.4. Useful buffers include sodium citrate-citric acid and sodium phosphate-phosphoric acid. A form of repository or “depot” slow release preparation may be used so that therapeutically effective amounts of the preparation are delivered into the bloodstream over many hours or days following parenteral injection.

The desired isotonicity of the formulations may be accomplished using sodium chloride or other pharmaceutically acceptable agents such as dextrose, boric acid, sodium tartrate, propylene glycol, polyols (such as mannitol and sorbitol), or other inorganic or organic solutes. Sodium chloride is preferred for buffers containing sodium ions.

If desired, solutions of the above compositions may be thickened with a thickening agent such as methylcellulose. They may be prepared in emulsified form, either water in oil or oil in water. Any of a wide variety of pharmaceutically acceptable emulsifying agents may be employed including, for example, acacia powder, a non-ionic surfactant, or an ionic surfactant, such as alkali polyether alcohol sulfates or sulfonates.

The pharmaceutical compositions may be prepared by mixing the ingredients following generally accepted procedures. For example, the selected components may be simply mixed in a blender or other standard device to produce a concentrated mixture which may then be adjusted to the final concentration and viscosity by the addition of water or thickening agent and possibly a buffer to control pH or an additional solute to control tonicity.

Typical therapeutically effective amounts of the amylin agonist compounds for use in parenteral formulations are from about 1 μg to about 5 mg; from about 10 μg to about 3 mg; from about 50 μg to about 2 mg; or from about 100 μg to about 1 mg. The dosages may be administered daily (e.g., BID or TID), weekly, or monthly. The therapeutically effective amount of the amylin agonist compound will depend on the formulation and frequency of administration (e.g., immediate release, sustained release). The therapeutically effective amount of the amylin agonist compounds for use in nasal or oral formulations may be about 5-fold to 15-fold greater than the amount used in parenteral formulations.

### Therapeutic Methods and Uses

In a general aspect, provided are methods of treating estrogen deficiency in mammals in need thereof by administering to the estrogen-deficient mammals therapeutically effective amounts of amylin agonist compounds or pharmaceutical compositions comprising amylin agonist compounds. Exemplary methods described herein include (i) treating obesity in estrogen-deficient mammals; (ii) treating overweight in estrogen-deficient mammals; (iii) reducing weight in estrogen-deficient mammals; (iv) reducing body fat in estrogen-deficient mammals; (v) reducing body fat while maintaining lean muscle mass in estrogen-deficient mammals; (vi) increasing satiety in estrogen-deficient mammals; (vii) reducing appetite in estrogen-deficient mammals; (viii) delaying gastric emptying in estrogen-deficient mammals; (ix) reducing gastric motility in estrogen-deficient mammals; (x) reducing ovariectomized weight gain and/or body fat in female mammals; (xi) treating ovariectomized obesity or overweight in female mammals; (xii) reducing menopausal weight gain and/or body fat in female humans; (xiii) reducing menopausal weight gain and/or body fat while maintaining lean muscle mass in female humans; (xiv) reducing postmenopausal weight gain and/or body fat in female humans; (xv) reducing postmenopausal weight gain and/or body fat while maintaining lean muscle mass in female humans; (xvi) reducing perimenopausal weight gain and/or body fat in female humans; (xvii) reducing postmenopausal weight gain and/or body fat while maintaining lean muscle mass in female humans; (xviii) treating menopausal obesity in female humans; (xix) treating perimenopausal obesity in female humans; (xx) treating postmenopausal obesity in female humans; (xxi) treating menopausal weight gain in female humans; (xxii) treating perimenopausal weight gain in female humans; (xxiii) treating postmenopausal weight gain in female humans; and (xxiv) increasing the levels of brain-derived neurotrophic factor (Bdnf) in estrogen-deficient mammals. The therapeutically effective amounts preferably provide at least a minimum therapeutically effective plasma level of the amylin agonist compounds in the mammals.

The methods described herein may further comprise improving glycemic control in the estrogen-deficient mammals by administering to the estrogen-deficient mammals therapeutically effective amounts of amylin agonist compounds or pharmaceutical compositions comprising amylin agonist compounds. Improving glycemic control includes lowering blood glucose levels, reducing hemoglobin A1c (HbA1c) levels, and the like. In one embodiment, the estrogen-deficient mammals have diabetes, such as type 2 diabetes, and are overweight or obese.

Within the context of the methods of treating estrogen deficiency and estrogen-deficient mammals mentioned above, the methods disclosed herein are used to increase the metabolic rate in an estrogen-deficient mammal, decrease a reduction in the metabolic rate in an estrogen-deficient mammal, or preserve the metabolic rate in an estrogen-deficient mammal. In certain embodiments, the metabolic rate may involve the preferential use of the body's fat as an energy source over lean body tissue. In one aspect, lean body mass is not decreased following administration of an amylin agonist as provided herein. In another aspect, a reduction in the lean body mass is lessened or prevented following administration of amylin agonist as provided herein. In still another aspect, lean body mass is increased following administration of an amylin agonist as provided herein. Such preference for fat as the energy source may be determined by comparing the amount of fatty tissue to lean body tissue, ascertained by measuring total body weight and fat content at the beginning and end of the treatment period. An increase in metabolic rate is a higher level of the use of calories or another energy source by an estrogen-deficient mammal over a period of time compared with the level of use of calories or other energy source by the estrogen-deficient mammal over another period of time under substantially similar or identical conditions without administration of an amylin agonist as provided herein. In certain embodiments, the metabolic rate is increased at least about 5% in an estrogen-deficient mammal, in other embodiments, the metabolic rate is increased at least about 10%, 15%, 20% 25%, 30%, or 35% in an estrogen-deficient mammal compared with the level of use of calories or other energy source by the estrogen-deficient mammal over another period of time under substantially similar or identical conditions without administration of an amylin agonist as provided herein. The increase in metabolic rate can be measured using a respiratory calorimeter, for example.

In another embodiment, the methods provided are effective to elicit a reduction of a decrease in metabolic rate in a an estrogen deficient mammal. Such a decrease in metabolic rate can be the primary result of estrogen deficiency experienced by an estrogen-deficient mammal, or may also be a secondary result from a nutritional or physical regimen engaged in by such an estrogen-deficient mammal, which leads to a reduction in metabolic rate, for example, due to a reduced calorie diet, a restricted diet, or weight loss. A restricted diet includes allowances or prohibitions, or both on the types of food or the amounts of food or both permitted in a diet, not necessarily based on calories. For example, as in individual diets, the body compensates with a reduced metabolic rate based on the lower caloric intake. In essence, the body down-regulates the requirement for food, thereby subsisting on less food. As dieting continues, the threshold for caloric intake is reduced. When dieting has ended, the individual typically gains weight while eating a normal diet because of the lowered caloric intake threshold and lower-basal metabolic rate (NIH Technology Assessment Conference Panel (1992) Ann. Intern. Med. 116:942-949; Wadden (1993) Ann. Intern. Med. 119:688-693). In one aspect, a method is provided to reduce the loss of metabolic rate in an estrogen-deficient mammal, where the loss of metabolic rate is the result of a reduced calorie diet or weight loss. By using such a method, the estrogen-deficient mammal's reduction in metabolic rate is decreased by at least about 10%, 15%, 20% 25%, 30%, 35%, 40%, 50%, 60%, 70%, 80%, 90%, or 95%. For such methods, it may be desirable to administer an amylin agonist as provided herein at the time the condition or nutritional or physical regimen is initiated which leads to a loss or reduction in metabolic rate. However, it is also contemplated that administration of an amylin agonist as provided herein is commenced before the condition or nutritional or physical regimen is initiated. In one instance, metabolic rate is measured using a respiratory calorimeter.

In another aspect, methods for reducing metabolic plateaus in an estrogen-deficient mammal are provided, where a method comprises administering an effective amount an amylin agonist as provided herein to such an estrogen-deficient mammal. In certain embodiments, the estrogen-deficient mammal is losing weight, or has lost weight, for example, due to a reduced calorie diet, increased exercise or a combination thereof By “metabolic plateau” is meant time intervals of steady metabolic rate while the body adjusts to changes in caloric or energy input. Changes in caloric input or expenditure can be the result of, for example, reduced calorie diets or increased physical activity. Such plateaus can be observed, for example, during a weight loss regimen when weight loss slows or stops. In certain embodiments, a method of the present invention reduces the duration of a metabolic plateau in an estrogen-deficient mammal compared with the duration of metabolic plateaus in an otherwise identical subject over the same period of time under substantially similar or identical conditions without administration of an amylin agonist as provided herein. In other embodiments, a method of the present invention reduces the frequency of metabolic plateaus compared with the frequency of metabolic plateaus in an otherwise identical estrogen-deficient mammal over the same period of time under substantially similar or identical conditions without administration of an amylin agonist as provided herein. In still other embodiments, a method of the present invention delays the onset of a metabolic plateau compared with the onset of a metabolic plateau in an otherwise identical estrogen-deficient mammal over the same period of time under substantially similar or identical conditions without administration of an amylin agonist as provided herein. In certain embodiments, metabolic plateaus are identified by charting periods of reduced or no weight loss. In certain embodiments, at least one metabolic plateau is reduced. In other embodiments, at least two, three, four, five, six, seven, eight, nine, or ten metabolic plateaus are reduced. In another aspect, metabolic plateaus are delayed one day as compared to an estrogen-deficient mammal not administered an amylin agonist as provided herein under identical or similar conditions. In other aspects, metabolic plateaus are delayed 2 days, 3 days, 4 days, 5 days, 6 days, 1 week, 10 days, 2 weeks or 3 weeks in an estrogen-deficient mammal.

In yet other embodiments, a method is provided to preserve the metabolic rate in an estrogen-deficient mammal. In certain embodiments, the estrogen-deficient mammal may be at risk of losing metabolic rate, for example, due to the initiation of a reduced calorie diet, restricted diet, or anticipated weight loss. A preservation of metabolic rate is a maintenance of the level of the use of calories or another energy source by an estrogen-deficient mammal over a period of time compared with the level of use of calories or other energy source by an otherwise identical estrogen-deficient mammal over the same period of time under substantially similar or identical conditions without administration of an amylin agonist as provided herein. In one aspect, the metabolic rate is maintained within 15% of the estrogen-deficient mammal's metabolic rate prior to the initiation of the event that results in the decrease in metabolic rate. In other aspects, the metabolic rate is maintained within 10%, within 7%, within 5%, within 3% or less of the estrogen-deficient mammal's metabolic rate. In one aspect, an amylin agonist as provided herein is administered at the initiation of a reduced calorie diet, restricted diet, or exercise regimen.

Metabolic rates can be assessed using any method available for determining such rates, for example by using a respiratory calorimeter. Such methods and devices for assaying metabolic rates are known in the art and are described, for example, in U.S. Pat. Nos. 4,572,208, 4,856,531, 6,468,222, 6,616,615, 6,013,009, and 6,475,158. Alternatively, the metabolic rate of an animal can be assessed by measuring the amount of lean tissue versus fatty tissue catabolized by the animal following the diet period. Thus, total body weight and fat content can be measured at the end of the dietary period. In rats, a frequently used method to determine total body fat is to surgically remove and weigh the retroperitoneal fat pad, a body of fat located in the retroperitoneum, the area between the posterior abdominal wall and the posterior parietal peritoneum. The pad weight is considered to be directly related to percent body fat of the animal. Since the relationship between body weight and body fat in rats is linear, obese animals have a correspondingly higher percent of body fat and retroperitoneal fat pad weight.

In another aspect of the present invention, methods for reducing fat mass by increasing the metabolic rate in an estrogen-deficient mammal are provided, where the methods comprise administering an amylin agonist as provided herein in an amount effective to reduce fat mass by increasing the estrogen-deficient mammal's metabolic rate. Fat mass can be expressed as a percentage of the total body mass. In some aspects, the fat mass is reduced by at least 1%, at least 5%, at least 10%, at least 15%, at least 20%, or at least 25% over the course of treatment. In one aspect, the estrogen-deficient mammal's lean mass is not decreased over the course of the treatment. In another aspect, the estrogen-deficient mammal's lean mass is maintained or increased over the course of the treatment. In another aspect, the estrogen-deficient mammal is on a reduced calorie diet or restricted diet. By “reduced calorie diet” is meant that the estrogen-deficient mammal is ingesting fewer calories per day than compared to the same estrogen-deficient mammal's normal diet. In one instance, the estrogen-deficient mammal is consuming at least 50 fewer calories per day. In other instances, the estrogen-deficient mammal is consuming at least 100, 150, 200, 250, 300, 400, 500, 600, 700, 800, 900, or 1000 fewer calories per day.

In certain embodiments of the present invention, a method for altering the fat distribution in an estrogen-deficient mammal is provided where the method comprises administering an amylin agonist as provide herein in an amount effective to alter fat distribution in the estrogen-deficient mammal. In one aspect, the alteration results from an increased metabolism of visceral or ectopic fat, or both in the estrogen-deficient mammal. In some embodiments, the method involves the metabolism of visceral or ectopic fat or both at a rate of at least about 5%, 10%, 15%, 20%, 25%, 30%, 40%, or 50% greater than for subcutaneous fat. In one aspect, the methods result in a favorable fat distribution. In certain embodiments, favorable fat distribution is an increased ratio of subcutaneous fat to visceral fat, ectopic fat, or both. In one aspect, the method involves an increase in lean body mass, for example, as a result of an increase in muscle cell mass.

In other embodiments, methods for reducing the amount of subcutaneous fat in an estrogen-deficient mammal are provided, wherein the method comprises administering, to an estrogen-deficient mammal in need thereof, an amylin agonist as provided herein in an amount effective to reduce the amount of subcutaneous fat in the estrogen-deficient mammal. In one instance, the amount of subcutaneous fat is reduced in an estrogen-deficient mammal by at least about 5%. In other instances, the amount of subcutaneous fat is reduced by at least about 10%, 15%, 20%, 25%, 30% 40%, or 50% compared to the estrogen-deficient mammal prior to administration of an amylin agonist as provided herein.

The methods described herein can be used to reduce the amount of visceral fat in an estrogen-deficient mammal. In one instance, the visceral fat is reduced in an estrogen-deficient mammal by at least about 5%. In other instances, the visceral fat is reduced in the estrogen-deficient mammal by at least about 10%, 15%, 20%, 25%, 30% 40%, or 50% compared to the estrogen-deficient mammal prior to administration of an amylin agonist as provided herein. Visceral fat can be measured through any means available to determine the amount of visceral fat in an estrogen-deficient mammal. Such methods include, for example, abdominal tomography by means of CT scanning and MRI. Other methods for determining visceral fat are described, for example, in U.S. Pat. Nos. 6,864,415, 6,850,797, and 6,487,445.

In certain embodiments, a method for preventing the accumulation of ectopic fat or reducing the amount of ectopic fat in an estrogen-deficient mammal is provided, wherein the method comprises administering, to an estrogen-deficient mammal in need thereof, an amylin agonist as provided herein in an amount effective to prevent accumulation of ectopic fat or to reduce the amount of ectopic fat in the estrogen-deficient mammal. In one instance, the amount of ectopic fat is reduced in an estrogen-deficient mammal by at least about 5% compared to the estrogen-deficient mammal prior to administration of an amylin agonist as provided herein. In other instances, the amount of ectopic fat is reduced in a estrogen-deficient mammal by at least about 10%, or by at least about 15%, 20%, 25%, 30% 40%, or 50%. Alternatively, the amount of ectopic fat is proportionally reduced 5%, 10%, 15%, 20%, 25%, 30%, 40%, 50%, 60%, 70%, 80%, 90%, or 100% in comparison to subcutaneous fat in an estrogen-deficient mammal. Ectopic fat can be measured in an estrogen-deficient mammal using any method available for measuring ectopic fat.

In other embodiments, methods are provided for producing a more favorable fat distribution in an estrogen-deficient mammal, where the method comprises administering to an estrogen-deficient mammal an amylin agonist as provided hereinin amounts effective to produce a favorable fat distribution. In certain embodiments, administration of an amylin agonist as provided herein reduces the amount of visceral fat or ectopic fat, or both, in an estrogen-deficient mammal. Such methods result in a higher ratio of subcutaneous fat to visceral fat or ectopic fat. Such improved ratios may result in a reduced risk of the development of cardiovascular diseases, polycystic ovary syndrome, metabolic syndrome, or any combinations thereof In certain embodiments, ectopic or visceral fat is metabolized at a rate 5% greater than subcutaneous fat. In other embodiments, ectopic or visceral fat is metabolized at a rate at least 10% 15%, 20%, 25%, 30% 50%, 60%, 70%, 80%, 90%, or 100% greater than subcutaneous fat.

Also provided are methods to reduce weight in a morbidly obese subject by first reducing the estrogen-deficient mammal's weight to a level below that of being morbidly obese, then administering to the estrogen-deficient mammal an amylin agonist as provided herein in an effective amount to further reduce the estrogen-deficient mammal's weight. Methods for reducing an estrogen-deficient mammal's weight to below that of morbid obesity include reducing caloric intake, increasing physical activity, drug therapy, bariatric surgery, such as gastric bypass surgery, or any combinations of the preceeding methods. In one aspect, administering an amylin agonist as provided herein further reduces the weight of the estrogen-deficient mammal. In other embodiments, methods are provided for reducing the body mass index in an estrogen-deficient mammal having a body mass index of 40 or less by administering an amylin agonist as provided hereinin effective amounts to further reduce the estrogen-deficient mammal's weight.

By reducing weight it is meant that the estrogen-deficient mammal loses a portion of his/her total body weight over the course of treatment, whether the course of treatment be days, weeks, months or years. Alternatively, reducing weight can be defined as a decrease in proportion of fat mass to lean mass (in other words, the estrogen-deficient mammal has lost fat mass, but maintained or gained lean mass, without necessarily a corresponding loss in total body weight). An effective amount of an amylin agonist as provided herein in these embodiments is an amount effective to reduce an estrogen-deficient mammal's body weight over the course of the treatment, or alternatively an amount effective to reduce the estrogen-deficient mammal's percentage of fat mass over the course of the treatment. In certain embodiments, the estrogen-deficient mammal's body weight is reduced, over the course of treatment, by at least about 1%, by at least about 5%, by at least about 10%, by at least about 15%, or by at least about 20%. Alternatively, the estrogen-deficient mammal's percentage of fat mass is reduced, over the course of treatment, by at least 1%, at least 5%, at least 10%, at least 15%, at least 20%, or at least 25%.

In certain embodiments, methods of reducing nutrient availability, e.g., reducing weight, in an estrogen-deficient mammal comprise administering to the estrogen-deficient mammal an effective amount of an amylin agonist as provided herein in a bolus dose one or more times a day. A bolus dose is an intermittent dosage of medicine (as opposed to a continuous infusion). An estrogen-deficient mammal can be administered one or more bolus doses per day. The bolus dose can be the same no matter when it is administered to the estrogen-deficient mammal, or can be adjusted such that the estrogen-deficient mammal is administered a larger bolus dose at certain times of the day as compared to others. Administration of an agent in certain formulations, e.g., sustained-release formulations, a bolus dose can be administered less frequently, for example, once every three days, once per week, twice a month, once every month. Furthermore, the time between bolus doses is preferably long enough to allow the drug administered in the previous bolus dose to clear the estrogen-deficient mammal's blood stream.

In other embodiments, methods of reducing nutrient availability, e.g., reducing weight, in an estrogen-deficient mammal comprise administering to the estrogen-deficient mammal an effective amount of an amylin agonist as provided herein in continuous doses. By continuous dose it is intended to mean the continuous infusion of the drug by, for example, intravenous injection or a transdermal patch. Alternatively, a continuous dose can be administered orally in the form of a controlled release capsule or tablet which releases the drug into the estrogen-deficient mammal's system over a period of time. When administered by a continuous dose, the drug is released over a period of about 1 hour, in some cases the drug is released over a period of about 2, 3, 4, 5, 6, 7, 8, 9, 10, 11, 12, 18, or 24 hours.

The present invention is yet further directed to methods of increasing oxidative metabolism in an estrogen-deficient mammal, the method comprising administering to an estrogen-deficient mammal in need thereof an effective amount of an amylin agonist as provided herein. Oxidative metabolism is the process by which oxygen is used to make energy from carbohydrates (sugars).

In another aspect, a method of inducing a feeling of fullness in an estrogen-deficient mammal is provided, wherein the method comprises administering an effective amount of an amylin agonist as provided herein to said estrogen-deficient mammal.

In yet another aspect, a method of controlling hunger in an estrogen-deficient mammal is provided, wherein the method comprises administering an effective amount of an amylin agonist as provided herein to said estrogen-deficient mammal.

In yet a further aspect, a method of prolonging a feeling of satiation in an estrogen-deficient mammal is provided, wherein the method comprises administering an effective amount of an amylin agonist as provided herein to said estrogen-deficient mammal.

In yet a further aspect, a method of reducing caloric intake of an estrogen-deficient mammal by reducing the size of a meal is provided, wherein the method comprises administering an effective amount of an amylin agonist as provided herein to said estrogen-deficient mammal.

In another aspect, a method of controlling food intake of an estrogen-deficient mammal is provided, wherein the method comprises administering an effective amount of an amylin agonist as provided herein to said estrogen-deficient mammal.

In yet another aspect, a method for ensuring or assisting in compliance of an estrogen-deficient mammal with a reduced calorie or restrictive diet is provided, wherein the method comprises administering an effective amount of an amylin agonist as provided herein to said estrogen-deficient mammal.

In a further aspect, a method of adjusting an estrogen-deficient mammal's set point so that the body's propensity for homeostasis is adjusted to a healthier set point is provided, wherein the method comprises administering an effective amount of an amylin agonist as provided herein to said estrogen-deficient mammal.

In yet a further aspect, a method is provided for maintaining weight loss or maintaining the weight lost in an estrogen-deficient mammal which has experienced or undergone or is undergoing a weight-reducing treatment therapy or regimen, wherein the method comprises administering an effective amount of an amylin agonist as provided herein to said estrogen-deficient mammal. In other embodiments of this aspect of the invention, the weight loss is maintained by re-setting the estrogen-deficient mammal's set point.

In certain embodiments, methods of the invention are of use in treating and/or preventing metabolic conditions or disorders that benefit from a reduction in nutrient availability in an estrogen-deficient mammal in need of such treatment or prevention. Accordingly, these methods may be useful in treating and/or preventing of obesity, diabetes (e.g., type 2 or non-insulin dependent diabetes, type 1 diabetes, and gestational diabetes), eating disorders, insulin-resistance syndrome, and cardiovascular disease.

In certain embodiments, methods of use in altering fat distribution, reducing fat mass, or both in an estrogen-deficient mammal are provided. Accordingly, subjects for whom altering body composition is of benefit can also benefit from the present methods. Altered body composition, as intended herein, includes loss or maintenance of body fat, with minimization of loss, maintenance, or gain of lean body mass. In such situations, weight may increase as well as decrease. Accordingly, subjects may be lean, overweight, or obese as these terms are generally used in the art. Methods of the invention may also include reducing fat in non-adipose tissue while sparing lean mass. Uses for this method include treating diseases such as nonalcoholic steatohepatitis (NASH) or lipodystrophy.

In other embodiments of the methods described herein, the estrogen-deficient mammal (e.g., female human) may further be administered an effective amount of leptin or a leptin analog. The leptin or leptin analog may be administered in the same pharmaceutical composition as the amylin agonist compound or may be administered in a separate pharmaceutical composition. In one embodiment, the leptin or leptin analog is metreleptin. The leptin and leptin analogs that may be employed in the methods disclosed herein are describe in, for example, U.S. Pat. No. 5,594,101, U.S. Pat. No. 5,851,995, U.S. Pat. No. 5,691,309, U.S. Pat. No. 5,580,954, U.S. Pat. No. 5,554,727, U.S. Pat. No. 5,552,523, U.S. Pat. No. 5,559,208, U.S. Pat. No. 5,756,461, U.S. Pat. No. 6,309,853, and PCT Published Application Nos. WO 96/23517, WO 96/005309, WO 2004/039832, WO 98/55139, WO 98/12224, and WO 97/02004, each of which is incorporated herein in its entirety and for all purposes. Any leptin or leptin analog known in the art may be used in accordinace with the disclosed methods. Representative leptins contemplated for use in the polypeptide conjugates and method described herein include the following leptins:

Murine leptin: VPIQKVQDDTKTLIKTIVTRINDISHT-Xaa-SVSSKQKVTGLDFIPGLHPILTLSKMDQTLAVYQQILTSMPSRNVIQISNDLENLRDLLHV LAFSKSCHLPQASGLETLESLGGVLEASGYSTEVVALSRLQGSLQDMLQQLDLSPGC, wherein Xaa at position 28 is Q or absent (SEQ ID NO: 3).

Porcine leptin: VPIWRVQDDTKTLIKTIVTRISDISHMQSVSSKQRVTGLDFIPGLHPVLSLSKMDQTLAIY QQILTSLPSRNVIQISNDLENLRDLLHLLASSKSCPLPQARALETLESLGGVLEASLYSTEV VALSRLQGALQDMLRQLDLSPGC (SEQ ID NO: 15).

Bovine leptin: VPICKVQDDTKTLIKTIVTRINDISHT-Xaa-SVSSKQRVTGLDFIPGLHPLLSLSKMDQTLAIYQQILTSLPSRNVVQISNDLENLRDLLHL LAASKSCPLPQVRALESLESLGVVLEASLYSTEVVALSRLQGSLQDMLRQLDLSPGC, wherein Xaa at position 28 is Q or absent (SEQ ID NO: 16).

Human leptin: VPIQKVQDDTKTLIKTIVTRINDISH-Xaa-Xaa-SVSSKQKVTGLDFIPGLHPILTLSKMDQTLAVYQQILTSMPSRNVIQISNDLENLRDLLHV LAFSKSCHLPWASGLETLDSLGGVLEASGYSTEVVALSRLQGSLQDMLWQLDLSPGC, wherein: Xaa at position 27 is T or A; and Xaa at position 28 is Q or absent (SEQ ID NO: 2).

Rhesus Leptin: VPIQKVQSDTKTLIKTIVTRINDISHTQSVSSKQRVTGLDFIPGLHPVLTLSQMDQTLAIYQ QILINLPSRNVIQISNDLENLRDLLHLLAFSKSCHLPLASGLETLESLGDVLEASLYSTEVV ALSRLQGSLQDMLWQLDLSPGC (SEQ ID NO: 17).

Rat leptin: VPIHKVQDDTKTLIKTIVTRINDISHTQSVSARQRVTGLDFIP GLHPILSLSKMDQTLAVYQQILTSLPSQNVLQIAHDLENLRDLLHLLAFSKSCSLPQTRGL QKPESLDGVLEASLYSTEVVALSRLQGSLQDILQQLDLSPEC (SEQ ID NO: 4).

Platypus leptin: The mature playpus leptin sequence follows: ISIEKIQADTKTLTKTIITRIIQLSTQNGVSTDQRVSGLDFIPGNQQFQNLADMDQTLAVYQ QILSSLPMPDRTQISNDLENLRSLFALLATLKNCPFTRSDGLDTMEIWGGIVEESLYSTEV VTLDRLRKSLKNIE KQLDHIQG (SEQ ID NO: 18). A full length sequence of platypus leptin, including a 21-residue N-terminal signal sequence follows: MRCILLYGFLCVWQHLYYSHPISIEKIQADTKTLTKTIITRIIQLSTQNGVSTDQRVSGLDFI PGNQQFQNLADMDQTLAVYQQILSSLPMPDRISNDLENLRSLFALLATLKNCPFTRSDGL DTMEIWGGIVEESLYSTEVVTLDRLRKSLKNI EKQLDHIQG (SEQ ID NO: 19).

Metreleptin (rmet-Hu-leptin; A100): MVPIQKVQDDTKTLIKTIVTRINDISHTQSVSSKQKVTGLDFIPGLHPILTLSKMDQTLAV YQQILTSMPSRNVIQISNDLENLRDLLHVLAFSKSCHLPWASGLETLDSLGGVLEASGYS TEVVALSRLQGSLQDMLWQLDLSPGC (SEQ ID NO:1).

Leptin A200: Leptin A200 is an Fc antibody fragment condensation product with leptin, as known in the art. See e.g., Lo et al., 2005, Protein Eng. Design & Selection, 18:1-10 (SEQ ID NO: 7).

Leptin A300: Leptin A300 is metreleptin with substitutions W101Q and W139Q (N-terminal *Met counted as residue 1): MVPIQKVQDDTKTLIKTIVTRINDISHTQSVSSKQKVTGLDFIPGLHPILTLSKMDQTLAV YQQILTSMPSRNVIQISNDLENLRDLLHVLAFSKSCHLPQASGLETLDSLGGVLEASGYST EVVALSRLQGSLQDMLQQLDLSPGC (SEQ ID NO:6).

Leptin A500: Research by a number of investigators including the inventors have focused on the effects on aggregation of residue substitution in leptin. See e.g., Ricci et al., 2006. “Mutational approach to improve physical stability of protein therapeutics susceptible to aggregation: Role of altered conformation in irreversible precipitation,” Book Chapter. In: Misbehaving Proteins: Protein (Mis)Folding, Aggregation, and Stability. Murphy R M, Tsai A M, Eds. New York. Springer, pp. 331-350, which is incorporated herein by reference and for all purposes. Accordingly, leptin A500 with sequence following has been used in the compounds and methods described herein: VPIQKVQDDTKTLIKTIVTRINDISHTQSVSSKQKVTGLEFIPGLHPILTLSKMDQTLAVYQ QILTSMPSRNVIQISNDLENLRDLLHVLAFSKSCHLPQASGLETLESLGGVLEASGYSTEV VALSRLQGSLQDMLQQLDLSPGC (SEQ ID NO:5).

Other exemplary leptins and leptin analogs and related molecules are reported in the following publications; however, no representation is made with regard to the activity of any composition reported: U.S. Pat. Nos. 5,521,283; 5,525,705; 5,532,336; 5,552,522; 5,552,523; 5,552,524; 5,554,727; 5,559,208; 5,563,243; 5,563,244; 5,563,245; 5,567,678; 5,567,803; 5,569,743; 5,569,744; 5,574,133; 5,580,954; 5,594,101; 5,594,104; 5,605,886; 5,614,379; 5,691,309; 5,719,266; PCT Publication Nos. WO96/23513; WO96/23514; WO96/23515; WO96/23516; WO96/23517; WO96/23518; WO96/23519; WO96/34111; WO96 37517; WO96/27385; WO97/00886; WO97/20933; WO97/16550; WO96/35787; WO96/34885; WO97/46585; WO96/22308; European Patent Publication Nos. EP 725078; EP 725079; EP 744408; EP 745610; EP 835879; EP 736599; EP 741187.

### Additional Embodiments

Embodiment 1. A method for treating estrogen deficiency in a mammal in need thereof comprising administering to the estrogen-deficient mammal a therapeutically effective amount of an amylin agonist compound.

Embodiment 2. A method for treating obesity in an estrogen-deficient mammal comprising administering to the estrogen-deficient mammal a therapeutically effective amount of an amylin agonist compound.

Embodiment 3. A method for treating overweight in an estrogen-deficient mammal comprising administering to the estrogen-deficient mammal a therapeutically effective amount of an amylin agonist compound.

Embodiment 4. A method for reducing weight in an estrogen-deficient mammal comprising administering to the estrogen-deficient mammal a therapeutically effective amount of an amylin agonist compound.

Embodiment 5. A method for reducing body fat in an estrogen-deficient mammal comprising administering to the estrogen-deficient mammal a therapeutically effective amount of an amylin agonist compound.

Embodiment 6. A method for reducing body fat while maintaining lean muscle mass in an estrogen-deficient mammal comprising administering to the estrogen-deficient mammal a therapeutically effective amount of an amylin agonist compound.

Embodiment 7. A method for reducing weight and/or body fat while maintaining lean muscle mass in an estrogen-deficient mammal comprising administering to the estrogen-deficient mammal a therapeutically effective amount of an amylin agonist compound.

Embodiment 8. A method for maintaining weight in an estrogen-deficient mammal comprising administering to the estrogen-deficient mammal a therapeutically effective amount of an amylin agonist compound.

Embodiment 9. A method for delaying gastric emptying in an estrogen-deficient mammal comprising administering to the estrogen-deficient mammal a therapeutically effective amount of an amylin agonist compound.

Embodiment 10. A method for reducing gastric motility in an estrogen-deficient mammal comprising administering to the estrogen-deficient mammal a therapeutically effective amount of an amylin agonist compound.

Embodiment 11. A method for reducing appetite in an estrogen-deficient mammal comprising administering to the estrogen-deficient mammal a therapeutically effective amount of an amylin agonist compound.

Embodiment 12. A method for increasing satiety in an estrogen-deficient mammal comprising administering to the estrogen-deficient mammal a therapeutically effective amount of an amylin agonist compound.

Embodiment 13. A method for reducing ovariectomized weight gain in a female mammal comprising administering to the estrogen-deficient mammal a therapeutically effective amount of an amylin agonist compound.

Embodiment 14. A method for treating ovariectomized obesity and/or overweight in a female mammal comprising administering to the estrogen-deficient mammal a therapeutically effective amount of an amylin agonist compound.

Embodiment 15. A method for reducing menopausal weight gain and/or body fat in a female mammal comprising administering to the menopausal female mammal a therapeutically effective amount of an amylin agonist compound.

Embodiment 16. A method for reducing menopausal weight gain and/or body fat while maintaining lean muscle mass in a female mammal comprising administering to the menopausal female mammal a therapeutically effective amount of an amylin agonist compound.

Embodiment 17. A method for reducing postmenopausal weight gain and/or body fat in a female mammal comprising administering to the postmenopausal female mammal a therapeutically effective amount of an amylin agonist compound.

Embodiment 18. A method for reducing postmenopausal weight gain and/or body fat while maintaining lean muscle mass in a female mammal comprising administering to the postmenopausal female mammal a therapeutically effective amount of an amylin agonist compound.

Embodiment 19. A method for reducing perimenopausal weight gain and/or body fat in a female mammal comprising administering to the perimenopausal female mammal a therapeutically effective amount of an amylin agonist compound.

Embodiment 20. A method for reducing perimenopausal weight gain and/or body fat while maintaining lean muscle mass in a female mammal comprising administering to the perimenopausal female mammal a therapeutically effective amount of an amylin agonist compound.

Embodiment 21. A method for treating menopausal obesity or overweight in a female mammal comprising administering to the menopausal female mammal a therapeutically effective amount of an amylin agonist compound.

Embodiment 22. A method for treating postmenopausal obesity or overweight in a female mammal comprising administering to the postmenopausal female mammal a therapeutically effective amount of an amylin agonist compound.

Embodiment 23. A method for treating perimenopausal obesity or overweight in a female mammal comprising administering to the perimenopausal female mammal a therapeutically effective amount of an amylin agonist compound.

Embodiment 24. A method for increasing Bdnf levels in an estrogen-deficient mammal in need thereof comprising administering to the estrogen-deficient mammal in need of increased Bdnf levels a therapeutically effective amount of an amylin agonist compound.

Embodiment 25. A method of increasing Bdnf levels in a mammal in need thereof comprising administering to the mammal in need of increased Bdnf levels a therapeutically effective amount of an amylin agonist compound.

Embodiment 26. The method of any one of Embodiments 1-25 further comprising administering an effective amount of leptin or a leptin analog.

Embodiment 27. The method of any one of Embodiments 1-25 further comprising administering an effective amount of metreleptin.

Embodiment 28. The method of any one of Embodiments 1-25 further comprising administering an effective amount of a GLP-1 receptor agonist or an analog thereof; PYY or an analog thereof; GIP or an analog thereof; or CCK or an analog thereof.

Embodiment 29. The method of Embodiment 28, wherein the GLP-1 receptor agonist is GLP-1(7-37) or exendin-4.

Embodiment 30. The method of any one of Embodiments 1-25, wherein the mammal is a female human.

Embodiment 31. The method of any one of Embodiments 1-25, wherein the amylin agonist compound is a compound of Formula (I).

Embodiment 32. The method of any one of Embodiments 1-25, wherein the amylin agonist compound is a compound of Formula (II).

Embodiment 33. The method of any one of Embodiments 1-25, wherein the amylin agonist compound is a compound of Formula (III).

Embodiment 34. The method of any one of Embodiments 1-25, wherein the amylin agonist compound is a compound of Formula (IV).

Embodiment 35. The method of any one of Embodiments 1-25, wherein the amylin agonist compound is a compound of Formula (V).

Embodiment 36. The method of any one of Embodiments 1-25, wherein the amylin agonist compound is a compound of Formula (VI).

Embodiment 37. The method of any one of Embodiments 1-25, wherein the amylin agonist compound comprises the amino acid sequence of any one of SEQ ID NOs:1-139 or a pharmaceutically acceptable salt thereof.

Embodiment 38. The method of any one of Embodiments 1-25, wherein the amylin agonist compound has at least 90% sequence identity to the amino acid sequence of any one of SEQ ID NOs:1-139.

Embodiment 39. The method of any one of Embodiments 1-25, wherein the amylin agonist compound comprises the amino acid sequence of any one of SEQ ID NOS: 1-11, 20-39, 138, and 139.

Embodiment 40. The method of any one of Embodiments 1-25, wherein the amylin agonist compound has at least 90% sequence identity to the amino acid sequence of any one of SEQ ID NOS: 1-11, 20-39, 138, and 139.

Embodiment 41. The method of any one of Embodiments 1-25, wherein the amylin agonist compound comprises the amino acid sequence of SEQ ID NO:20 or a pharmaceutically acceptable salt thereof.

Embodiment 42. The method of any one of Embodiments 1-25, wherein the amylin agonist compound comprises the amino acid sequence of any one of SEQ ID NOS:40-137 or a pharmaceutically acceptable slat thereof.

Embodiment 43. The method of any one of Embodiments 1-25, wherein the amylin agonist compound has at least 90% sequence identity to the amino acid sequence of any one of SEQ ID NOS:40-137.

Embodiment 44. The method of any one of Embodiments 1-25, wherein the amylin agonist compound comprises the amino acid sequence of SEQ ID NO:137.

Embodiment 45. The method of any one of Embodiments 1-25, wherein the amylin agonist compound is peripherally administered to the mammal.

Embodiment 46. The method of any one of Embodiments 1-25, wherein the amylin agonist compound is parenterally administered to the mammal.

Embodiment 47. The method of any one of Embodiments 1-25, wherein the amylin agonist compound is subcutaneously administered to the mammal.

Embodiment 48. The method of any one of Embodiments 1-25, wherein the amylin agonist compound is intravenously administered to the mammal.

Embodiment 49. The method of any one of Embodiments 1-25, wherein the amylin agonist compound is intramuscularly administered to the mammal.

Embodiment 50. The method of any one of Embodiments 1-25, wherein the amylin agonist compound is intraarterialy administered to the mammal.

Embodiment 51. The method of any one of Embodiments 1-25, wherein the amylin agonist compound is nasally administered to the mammal.

Embodiment 52. The method of any one of Embodiments 1-25, wherein the amylin agonist compound has an IC₅₀ of about 200 or less in an amylin receptor binding assay.

Embodiment 53. The method of any one of Embodiments 1-25, wherein the amylin agonist compound has an IC₅₀ of about 100 or less in an amylin receptor binding assay.

Embodiment 54. The method of any one of Embodiments 1-25, wherein the amylin agonist compound has an IC₅₀ of about 50 or less in an amylin receptor binding assay.

Embodiment 55. The method of any one of Embodiments 1-25, wherein the amylin agonist compound has an EC₅₀ of about 20 or less in soleus muscle assay.

Embodiment 56. The method of any one of Embodiments 1-25, wherein the amylin agonist compound has an EC₅₀ of about 15 or less in soleus muscle assay.

Embodiment 57. The method of any one of Embodiments 1-25, wherein the amylin agonist compound has an EC₅₀ of about 5 or less in soleus muscle assay.

Embodiment 58. The method of any one of Embodiments 1-25, wherein the amylin agonist compound is linked to a peptide, a carbohydrate, a saccharide, polyethylene glycol, albumin, a fatty acid, a polyamino acid, dextran, gelatin, a polyvinyl pyrrolidone, a polyvinyl alcohol, an N-(2-hydroxypropyl)-methacrylamide, or a combination of two or more thereof.

Embodiment 59. The method of any one of Embodiments 1-25, wherein the amylin agonist compound is linked to a peptide, a carbohydrate, a saccharide, a polyethylene glycol, albumin, a fatty acid, or a polyamino acid.

Embodiment 60. The method of Embodiment 58 or 59, wherein the peptides is an exendin, an exendin analog, a GLP-1, a GLP-1 analog, a CCK, a CCK analog, a PYY, a PYY analog, a GIP, or a GIP analog.

Embodiment 61. The method of any one of Embodiments 1-25, wherein the amylin agonist compound is glycosylated at one, two, three, or four, amino acid residues.

Embodiment 62. The method of any one of Embodiments 1-25, wherein the mammal is not being administered hormone replacement therapy.

Embodiment 63. The method of any one of Embodiments 1-25, wherein the mammal is perimenopausal.

Embodiment 64. The method of any one of Embodiments 1-25, wherein the mammal is menopausal.

Embodiment 65. The method of any one of Embodiments 1-25, wherein the mammal is postmenopausal.

Embodiment 66. The method of any one of Embodiments 1-25, wherein the mammal has an ovarian dysfunction, or has had an overectomy or a hysterectomy.

Embodiment 67. The method of any one of Embodiments 30, wherein the female human has an estradiol level of about 30 or less and a follicle stimulating hormone level of about 30 or more.

Embodiment 68. The method of any one of Embodiments 1-25 wherein the therapeutically effective amount of the amylin agonist compound is from 1 μg to 5 mg.

Embodiment 69. The method of any one of Embodiments 1-25 wherein the therapeutically effective amount of the amylin agonist compound is from 1 μg/day to 5 mg/day.

Embodiment 70. The method of any one of Embodiments 1-25, wherein the therapeutically effective amount of the amylin agonist compound is from 1 μg/week to 5 mg/week.

Embodiment 71. The method of any one of Embodiments 1-25, wherein the therapeutically effective amount of the amylin agonist compound is from 1 μg/month to 5 mg/month.

Embodiment 72. The method of any one of Embodiments 1-25 wherein the therapeutically effective amount of the amylin agonist compound is from 10 μg to 3 mg.

Embodiment 73. The method of any one of Embodiments 1-25 wherein the therapeutically effective amount of the amylin agonist compound is from 10 μg/day to 3 mg/day.

Embodiment 74. The method of any one of Embodiments 1-25, wherein the therapeutically effective amount of the amylin agonist analog is from 10 μg/week to 3 mg/week.

Embodiment 75. The method of any one of Embodiments 25, wherein the therapeutically effective amount of the amylin agonist analog is from 10 μg/month to 3 mg/month.

Embodiment 76. The method of any one of Embodiments 1-25, wherein the therapeutically effective amount of the amylin agonist compound is from 50 μg to 2 mg.

Embodiment 77. The method of any one of Embodiments 1-25, wherein the therapeutically effective amount of the amylin agonist compound is from 50 μg/day to 2 mg/day.

Embodiment 78. The method of any one of Embodiments 1-25 wherein the therapeutically effective amount of the amylin agonist compound is from 50 μg/week to 2 mg/week.

Embodiment 79. The method of any one of Embodiments 1-25, wherein the therapeutically effective amount of the amylin agonist compound is from 50 μg/month to 2 mg/month.

Embodiment 80. The method of any one of Embodiments 1-25, wherein the therapeutically effective amount of the amylin agonist compound is from 100 μg to 1 mg.

Embodiment 81. The method of any one of Embodiments 1-25, wherein the therapeutically effective amount of the amylin agonist compound is from 100 μg/day to 1 mg/day.

Embodiment 82. The method of any one of Embodiments 1-25, wherein the therapeutically effective amount of the amylin agonist analog is administered daily in a single or divided dose.

Embodiment 83. The method of any one of Embodiments 1-60 wherein the therapeutically effective amount of the amylin agonist analog is from 100 μg/week to 1 mg/week.

Embodiment 84. The method of any one of Embodiments 1-60 wherein the therapeutically effective amount of the amylin agonist analog is from 100 μg/month to 1 mg/month.

Embodiment 85. The method of any one of Embodiments 1-25, wherein the therapeutically effective amount of the amylin agonist analog provides at least a therapeutically effective minimum plasma level of the amylin agonist compound.

Embodiment 86. The method of any one of Embodiments 1-25, wherein the amylin agonist compound is a compound of Formula (VII).

Embodiment 87. The method of any one of Embodiments 1-25, wherein the amylin agonist compound is a compound of Formula (VIII).

Embodiment 88. The method of any one of Embodiments 1-25, wherein the amylin agonist compound is a compound of Formula (IX).

Embodiment 89. The method of any one of Embodiments 1-25, wherein the amylin agonist compound is a compound of Formula (X).

Embodiment 90. The method of any one of Embodiments 1-25, wherein the amylin agonist compound comprises a fragment of a human amylin amino acid sequence, a fragment of a rat amino acid sequence, a fragment of a salmon calcitonin amino acid sequence, a fragment of a human calcitonin sequence, or a combination of two or more thereof.

Embodiment 91. The method of any one of Embodiments 1-25, wherein the amylin agonist compound comprises (a) a loop region; (b) an α helix loop type I; and (c) a C-terminal tail.

Embodiment 92. The method of any one of Embodiments 1-25, wherein the amylin agonist compound comprises (i) an amylin peptide fragment and (ii) a calcitonin peptide fragment.

Embodiment 93. The method of Embodiment 92, wherein the amylin is human amylin or rat amylin; and the calcitonin is human calcitonin or salmon calcitonin.

Embodiment 94. The method of any one of Embodiments 1-25, wherein the mammal has diabetes.

Embodiment 95. The method of any one of Embodiments 1-14 and 94, which further comprises improving glycemic control in the estrogen-deficient mammal in need thereof.

Embodiment 96. The method of Embodiment 95, wherein the method of improving glycemic control is a method of lowering blood glucose levels or reducing hemoglobin A1c (HbA1c) levels.

Embodiment 97. The method of any one of Embodiments 15-24 and 94, which further comprises improving glycemic control in the menopausal, postmenopausal, or perimenopausal female mammal in need thereof.

Embodiment 98. The method of Embodiment 97, wherein the method of improving glycemic control is a method of lowering blood glucose levels or reducing hemoglobin A1c (HbA1c) levels.

Embodiment 99. The method of any one of Embodiments 1-98, wherein the amylin agonist compound comprises the amino acid sequence of SEQ ID NO:142 or a pharmaceutically acceptable salt thereof.

### EXAMPLES

The following examples are for purposes of illustration only and are not intended to limit the scope of the disclosure or the claims.

### Example 1

The amylin receptor binding assay, a competition assay that measures the ability of compounds to bind specifically to membrane-bound amylin receptors, is described in U.S. Pat. No. 5,686,411 (e.g., Example 18), the disclosure of which is incorporated by reference herein. A preferred source of the membrane preparations used in the assay is the basal forebrain which comprises membranes from the nucleus accumbens and surrounding regions. Compounds being assayed compete for binding to these receptor preparations with ¹²⁵I Bolton Hunter rat amylin. Competition curves, wherein the amount bound (B) is plotted as a function of the log of the concentration of ligand, are analyzed by computer using analyses by nonlinear regression to a 4-parameter logistic equation (Inplot program; GraphPAD Software, San Diego, Calif.) or the ALLFIT program of DeLean et al. (ALLFIT, Version 2.7 (NIH, Bethesda, Md. 20892)). Munson and Rodbard, Anal. Biochem. 107:220-239 (1980).

### Example 2

Assays of biological activity of amylin agonist compounds in the soleus muscle may be performed using methods described in U.S. Pat. No. 5,686,411 (e.g., Example 19), the disclosure of which is incorporated by reference herein, in which amylin agonist activity may be assessed by measuring the inhibition of insulin-stimulated glycogen synthesis. In brief, an exemplary method includes soleus muscle strips prepared from 12-h fasted male Wistar rats. The tendons of the muscles are ligated before attachment to stainless steel clips. Muscle strips are pre-incubated in Erlenmeyer flasks containing 3.5 ml Krebs-Ringer bicarbonate buffer, 7 mM N-2-hydroxyethyl-peperazine-N′-2-ethane-sulphonic acid, pH 7.4, and 5.5 mM pyruvate. Flasks are sealed and gassed continuously with O₂ and CO₂ in the ratio 19:1 (v/v). After pre-incubation of muscles in this medium for 30 min at 37° C. in an oscillating water bath, the muscles strips are transferred to similar vials containing identical medium (except pyruvate) with added [U-¹⁴C] glucose (0.5 μCi/ml) and insulin (100 μU/ml). The flasks are sealed and re-gassed for an initial 15 min in a 1-h incubation. At the end of the incubation period, muscles are blotted and rapidly frozen in liquid N₂. The concentration of lactate in the incubation medium can be determined spectrophotometrically and [U-¹⁴C]glucose incorporation in glycogen measured.

All studies described in the Examples below were approved by the Institutional Animal Care and Use Committee at Amylin Pharmaceuticals, Inc., in accordance with Animal Welfare Act guidelines. Animals were housed in standard caging at 22° C. on a 12-hour light, 12-hour dark cycle. Additionally, all data were analyzed using a one- or two-way analysis of variance (ANOVA) with Neuman-Keuls or Bonferroni post hoc analyses respectively. Statistical significance was assumed for p<0.05. Graphs were generated using Prism 4 for Windows (Graphpad Software, San Diego, Calif.). All data are presented as mean±SEM.

### Example 3

### Materials and Methods.

Female obesity-prone rats (n=36; CRL:CD-OP, Charles River, Wilmington, Mass.) were pre-fattened for approximately 4 months on 32% kcal/fat purified high fat diet (D12366B, Research Diets, New Brunswick, N.J.) to induce obesity in this model. Rats were then weight-matched (mean body weight 311±4 g) and subjected to either bilateral ovariectomy surgery (OVX; n=24), or sham surgery (SHAM; n=12). Rats were allowed approximately 3 weeks to recover from surgery, and after one week recovery a cohort of the OVX group (n=12) began to receive estrogen replacement (OVX+E; 2 ug β-estradiol (Sigma-Aldrich, St Louis, Mo.) in peanut oil subcutaneously every four days). Approximately 3 weeks post-surgery, half of each group had a single osmotic minipump (Alzet model 2ML4; Durect Corporation, Cupertino, Calif.) implanted subcutaneously delivering either vehicle (50% dimethylsulfoxide in sterile water), or rat amylin (50 ug/kg/d, Amylin Pharmaceuticals, Inc., San Diego, Calif.) for 28 days. Food intake and body weight were measured weekly. Body composition was assessed on day −1 and at termination by NMR (Echo Medical Systems, Houston, Tex.). On day 28 animals were euthanized by isoflurane overdose and cardiac blood collected.

### Results.

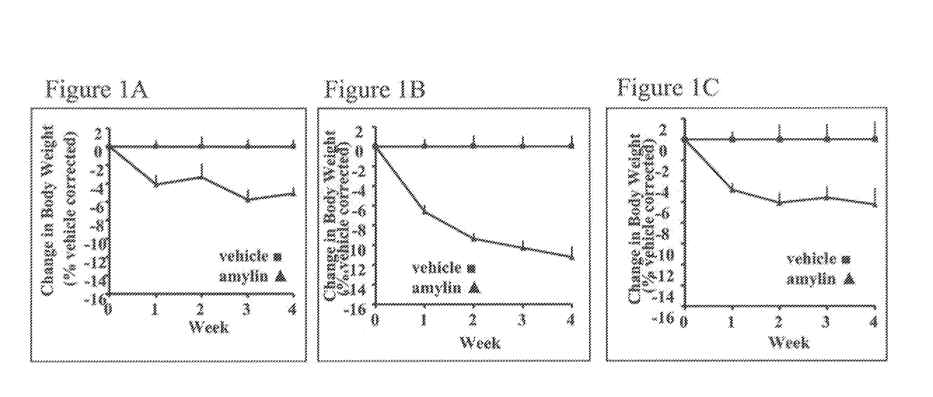

In the sham-treated female rats (SHAM), sustained infusion of rat amylin (SEQ ID NO: 15) (50 μg/kg/day for 4 weeks) induced sustained vehicle-corrected weight loss of 5.1±1.1% (FIG. 1A), in agreement with previous studies in male DIO rats at this dose and timepoint. In a group of twelve bilateral-ovariectomized (OVX) female rats, sustained infusion of rat amylin (SEQ ID NO: 15) (50 μg/kg/day for 4 weeks) induced sustained weight loss of 11.2±1.1% same dose of amylin in OVX rats, however, induced −11.2±1.1% body weight loss (p<0.001 from weeks 2-4 vs. SHAM; FIG. 1B). A second group of twelve OVX rats, which were treated with a sustained infusion of 17-β estradiol (OVX-E) at a dosage of 2 μg SC every 4 days in addition to being administered sustained infusion of rat amylin (SEQ ID NO:15) (50 μg/kg/day) throughout a 4-week period, sustained weight loss of 6.3±1.6% (p<0.01 weeks 3-4 vs. OVX group; ns vs. SHAM at any timepoint; FIG. 1C); thus, replacement of estrogen in OVX rats (OVX+E) abrogated the effect with amylin infusion observed in OVX rats in which no estrogen was administered.

The observed effect of amylin on body weight in the estrogen-deficient state described above is better understood by accounting for the effect of OVX surgery itself on promoting obesity. Body weight gain in grams over the 28 day treatment period was: SHAM—vehicle, 18.5±2.3 g; OVX—vehicle, 30.2±3.3 g; and OVX+E—vehicle, 22.2±4.2 g (p<0.05 for OVX vs. SHAM, but not OVX+E, at week 4; not shown). For amylin-treated groups the change in body weight in grams was: SHAM—amylin, 2.4±2.1 g; OVX—amylin, −7.7±3.6 g; and OVX+E, 3.6±4.4 g (p<0.05 for OVX vs. OVX+E, but not SHAM, at week 4; not shown). These data suggest that although obese OVX rats demonstrated susceptibility to weight gain over the treatment period, amylin infusion not only counteracted this propensity, but actively induced body weight loss below baseline levels.

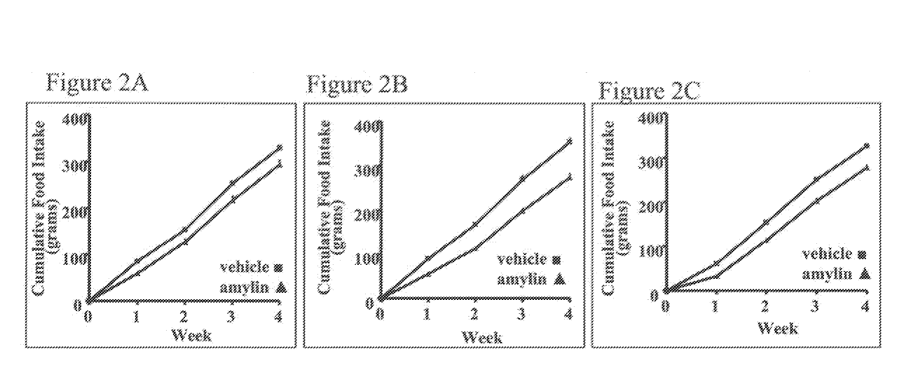

Amylin infusion in these rats was associated with reduced cumulative food intake relative to vehicle controls across all surgical groups. Specifically: the SHAM rats that received amylin infusion experienced a reduction in cumulative food intake of 10.9±3.4% relative to SHAM rats that received vehicle only (FIG. 2A); the OVX rats that received amylin infusion experienced a reduction of cumulative food intake of 23.0±2.0% relative to OVX rats that received vehicle only (FIG. 2B); and the OVX+E rats that received amylin infusion experienced a reduction in cumulative food intake of approximately 15% relative to OVX+E rats that received vehicle only (FIG. 2C). The amylin-induced reduction in cumulative food intake was not enhanced in the OVX group relative to either the SHAM group or the OVX+E group. Additionally, OVX vehicle-treated animals consumed more food than both SHAM vehicle-treated animals and OVX+E vehicle treated animals at week 4 (SHAM vehicle-treated, 329±7 g; OVX vehicle-treated, 354±11 g; OVX+E vehicle-treated, 326±7 g; p<0.05 for OVX vs. SHAM and OVX+E groups at week 4); however, there was no difference in cumulative food intake between amylin-treated groups.

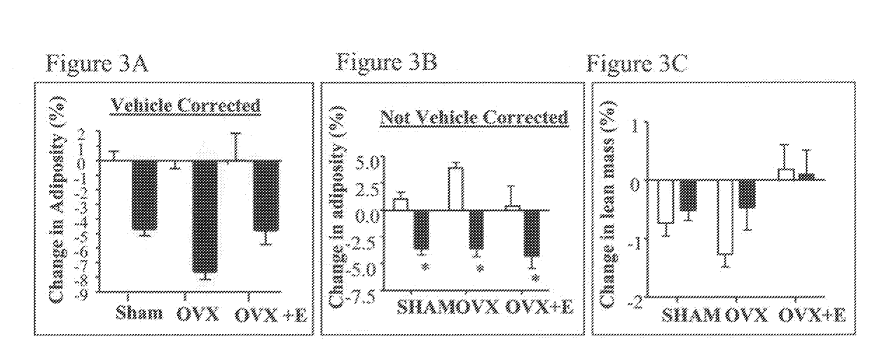

Amylin treatment was also associated with significant reductions in adiposity (percent fat mass) as depicted in FIGS. 3A (vehicle corrected) and 3B (not vehicle corrected). In the OVX female rats, amylin infusion induced a reduction in adiposity of about 7.5±0.6% compared to vehicle. In the OVX+E female rats; , amylin infusion induced a reduction in adiposity of about 4.7% compared to vehicle. When the data is view without correcting for vehicle (FIG. 3B) As reflected in the non-vehicle corrected presentation of the dataset, as in FIG. 3B, there appears to be no effect of surgery, or an interaction between drug and surgery, or change in adiposity (compare unfilled bars in FIG. 3B), despite a trend for an increase in OVX—vehicle controls (see filled basr, FIG. 3B). There was also no effect of drug or surgery on change in percent lean mass (FIG. 3C).

Fhus, as summarized in part in Table 1, below, results observed after four weeks of treatment with amylin, demonstrated that OVX female rats experienced superior weight loss, superior reduction in food intake, and superior reduction in adiposity when compared to sham-treated female rats (SHAM) and OVX female rats administered estrogen (OVX+E). The approximately 2-fold observed increase in amylin's efficacy in inducing weight loss, reduction in cumulative food intake, and decrease in adiposity in OVX female rats was reversed in OVX female rats administered 17-β estradiol back to an equivalent level observed in SHAM rats.

TABLE 1

| Amylin | | Reduction | Reduction in |
|---|---|---|---|
| administration | Sustained Weight | in Food | Adiposity |
| 50 μg/kg/d | Loss after 4 weeks | Intake | (Vehicle Corrected) |
| Sham Rats | 5.1 ± 1.1% | 10.9 ± 3.4% | 4.6 ± 0.5% |
| OVX Rats | 11.2 ± 1.9% | 23.0 ± 2.0% | 7.5 ± 0.6% |
| OVX + E Rats | 6% | 15% | 4.7% |

(p < 0.05 vs. all groups)

The decreased adiposity was associated with a significant reduction in leptin levels in all amylin-treated groups. Amylin also tended to reduce insulin, in all groups, and reduced glucose in SHAM but not OVX or OVX+E rats. Levels of the orexigenic hormone ghrelin were unaltered by amylin treatment at 28 days.

To explore the central mechanisms whereby exogenous amylin elicited enhanced weight loss in the OVX state compared to SHAM or OVX+E animals, mediobasal hypothalamic gene expression was examined. Levels of brain-derived neurotrophic factor (Bdnf) mRNA, a key neurotropin that can act as an anorexigenic neuropeptide highly expressed in the ventromedial hypothalamus, were increased (1.7-fold) in amylin-treated OVX rats compared to SHAM or OVX+E animals. No difference in the expression of key neuropeptides proopiomelanocortin (Pomc), neuropeptide Y (Npy), or agouti-related peptide (Agrp), or of estrogen receptor-α (ERα), were observed. These data imply a role for estrogen in the modulation of amylin signaling, the mechanisms for which may involve Bdnf function.

### Example 4

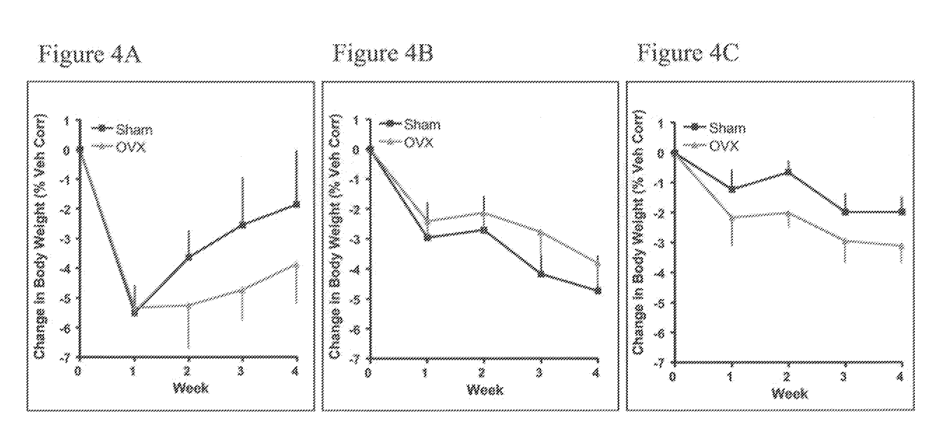

Female obesity-prone rats were treated as described above in Example 3 to generate another set of SHAM, OVX, and OVX+E rats. Each group of rats was administered either vehicle or one of the following amylin agonists: 50 μg/kg/day of SEQ ID NO: 20; 2 μg/kg/day of SEQ ID NO: 137; or 5 μg/kg/day of SEQ ID NO: 142. The results, depicted in FIGS. 4A, 4B, and 4C, demonstrate that, each of these amylin agonists tested exhibited either enhanced similar weight loss (taking into account the standard deviation of the datapoints) when administered to OVX rats relative to the weight loss observed when administered to the SHAM rats. Thus, consistent with the observations described in Example 3, these exemplary amylin agonists at least attenuate typical weight gain that is associated with estrogen deficiency and can even elicit enhanced weight loss in the setting of estrogen deficiency relative to the SHAM contrils, which have normal estrogen levels.

### Example 5

Enhanced Body Weight Loss Induced by Amylin Infusion is Associated with Improved Metabolism in OVX Rats.

### Materials and Methods.

To further characterize the apparent enhanced sensitivity to amylin-mediated weight loss in the OVX state the metabolism of SHAM or OVX rats treated with vehicle or amylin during days 1-4 of treatment was monitored.

A similar experimental design to that described in Examples 3 and 4 was followed. Female CRL:CD-OP rats (n=24) were pre-fattened for approximately 4 months (mean body weight 295±3 g), and then subjected to either SHAM (n=12) or OVX (n=12) surgery. Approximately 3 weeks post-surgery rats were implanted with a single osmotic minipump (Alzet model 2ML2, Durect Corporation) delivering either vehicle (n=6 per surgical group), or amylin (50 ug/kg/d; n=6 per surgical group). Animals were acclimated to the metabolic cages for 3 hr the day before pump implant, and were returned to the same chamber the morning after pump implant surgery. Rats were housed in the metabolic chambers for 2 nights and rates of oxygen consumption (VO₂), a proxy for metabolic rate, and carbon dioxide production (VCO₂) were measured using a high-speed Oxymax indirect calorimetry system (Columbus Instruments, Columbus, Ohio). Metabolic rate was normalized for initial body weight of the animal. Respiratory quotient (RQ) was calculated as VCO₂/VO₂. Total physical activity in the X-axis (laser beam breaks) was also measured throughout the experiment. An automated feeding system apparatus allowed accurate continuous monitoring of food intake. After two nights, rats were returned to home caging and utilized for assessment of neurogenesis (described below).

The effect of amylin infusion to OVX rats on energy metabolism compared to a yoked-fed control group was also assessed. For this study, female CRL:CD-OP rats (n=24) were pre-fattened for approximately 4 months (mean body weight 348±4 g), and then subjected to OVX surgery. Approximately 2 weeks post-surgery rats were implanted with a single osmotic minipump (Alzet model 2ML1, Durect Corporation) delivering either vehicle (n=16), or amylin (50 ug/kg/d; n=8). Animals were acclimated to the metabolic cages for 3 hr the day before pump implant, and were returned to the same chamber the morning after pump implant surgery. Rats were housed in the metabolic chambers for 5 nights and VO₂ and VCO₂ normalized to initial body weight were measured using a high-speed Oxymax indirect calorimetry system (Columbus Instruments). Total physical activity in the X-axis (laser beam breaks) was also measured. An automated feeding system restricted the amount of food allowed to half the vehicle group such that they consumed the mean amount of food consumed by the amylin-treated animals every 30 mins. In effect, this control group is therefore truly food-matched, or yoked-fed, as they consumed the same amount of food at approximately the same time as the test (amylin-treated) group, rather than in a traditional pair-fed group where animals consume their daily allotment of food quickly after presentation.

### Results.

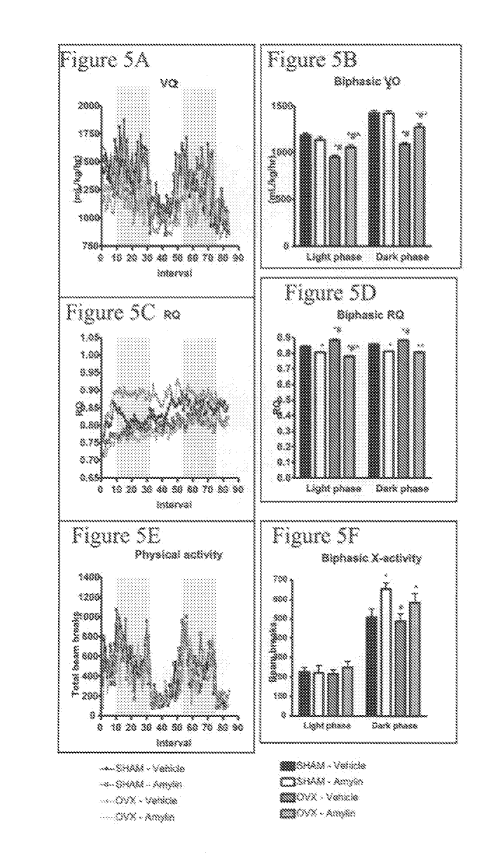

During the period in the metabolic chambers body weight change for each group was: SHAM—vehicle, 9.8±4.7 g; SHAM—amylin, 5.4±3.0 g; OVX—vehicle, 9.2±5.4 g; OVX—amylin, 6.4±1.4 g (no significant difference between groups). At these early timepoints body weights had not significantly diverged from baseline. We were able to continuously monitor metabolic rate (as VO₂), substrate utilization (as RQ) and physical activity in the X-axis (as beam breaks) for the duration of this experiment. Metabolic rate was not altered by amylin administration in SHAM-treated DIO female rats, in agreement with amylin effects on metabolism in male DIO rats (15). OVX surgery, however, significantly reduced VO₂ during both the light and dark phases (FIGS. 5A and 5B). OVX—vehicle controls exhibited reduced VO₂ compared to both SHAM—vehicle and SHAM—amylin groups. Amylin administration to OVX females significantly increased VO₂ but metabolic rate remained lower compared to SHAM groups (FIG. 5B). RQ values were significantly reduced by amylin administration in SHAM animals (FIGS. 5C and 5D), indicating a preference for fat utilization. OVX surgery resulted in an increase in RQ relative to both SHAM groups, however amylin treatment also reduced RQ in the OVX state, during the light phase RQ was lowered even beyond that of SHAM—amylin group (FIG. 5D). During the light phase there was no difference in physical activity between the groups, however during the dark phase when overall activity markedly increased, amylin treatment was associated with increased activity in both the SHAM and OVX state (FIGS. 5E and 5F). Taken together, OVX was associated with reduced metabolic rate and reduced preference for utilizing fat relative to SHAM controls, and amylin administration to OVX females exerted pro-metabolic consequences including increasing metabolic rate and fat oxidation, as well as physical activity, that likely contribute to the overall enhanced weight loss observed in this model.

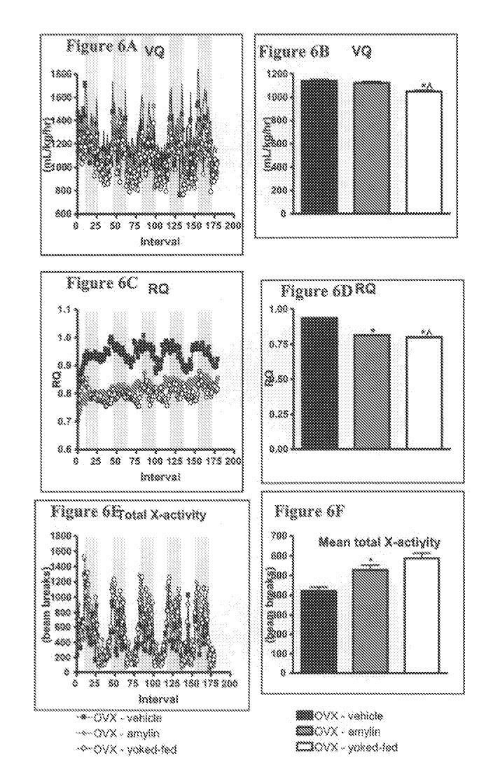

The metabolic consequence of amylin administration to OVX female DIO rats relative to vehicle controls, as well as yoked-fed controls (OVX—YF) during days 1-6 of treatment was next examined. During the period in the metabolic chambers food intake was matched between the OVX—amylin (50.1±6.7 g) and OVX—YF groups (43.4±0.9 g; both p<0.05 vs. OVX—vehicle controls, 82.0±3.9 g), and body weight was reduced by amylin treatment and yoked feeding: OVX—vehicle, 25.0±2.4 g; OVX—amylin, 4.5±6.6 g; OVX—YF, −3.2±2.3 g (p<0.05 for amylin and YF groups vs. vehicle). Similar to the previous experiment amylin administration had no significant effect on VO₂ levels, however OVX—YF rats demonstrated reduced VO₂ compared to OVX—vehicle and OVX—amylin groups (FIGS. 6A and 6B). A profound reduction in RQ was again evident with both amylin treatment and YF rats, which was modestly but significantly lower relative to the OVX—amylin group (FIGS. 6C and 6D). Total physical activity levels were increased by amylin treatment compared to vehicle controls, with OVX—YF rats also exhibiting similar increased activity (FIGS. 3E and 3F).

### Example 6

### Central Mechanisms Underlying Amylin-Mediated Exaggerated Weight Loss in Estrogen-Deficient Obese Rats.

### Materials and Methods.

Assessment of neuronal activation in DIO estrogen-deficient rats following an acute amylin challenge. Twenty female rats underwent either OVX or SHAM surgery as described in the above Examples. After recovering for 2 weeks, animals were then injected with amylin (10 μg/kg IP; Peptisyntha, Torrance, Calif.; n=5 OVX and 5 SHAM) or saline (n=5 OVX and 5 SHAM) beginning at 2 hours into the light cycle. Two hours later, rats were fully anesthetized and were perfused with saline followed by cold 4% PFA. Tissue was postfixed overnight and cryoprotected in 30% sucrose for 24 hours. Brains were frozen on dry ice and stored at −80° C. until sectioning, when five series of 30 μm thick sections were cut on a freezing microtome.

Immunohistochemistry was performed on free-floating sections. For c-Fos, blocking was performed with 1% bovine serum albumin (BSA). Rabbit polyclonal antibody against amino acids 210-335 of human c-Fos protein that are not cross-reactive with FosB, Fra-1, and Fra-2 (Santa Cruz Biotechnology, Santa Cruz, Calif.) were used in a dilution of 1:4,000. Sections were incubated overnight at 4° C. The next day tissue was incubated with goat anti-rabbit Alexa-Fluor 488 secondary antibody (1:250, Molecular Probes, Eugene, Oreg.) in PBS/Triton for 2 hr in the dark. After washing with PBS, tissue was mounted immediately onto slides and coverslipped with Vectashield Prolong Gold antifade reagent (Molecular Probes, Eugene, Oreg.) and stored at −20° C. Fluorescent images were captured using a Leica LSM 710 confocal microscope and Zen 2008 software. Analysis of positive cells in each region was conducted by a blinded investigator, and counting of c-Fos-positive nuclei was performed manually. The results are expressed as the mean number of cells per 3 sections per animal.

Assessment of Neurogenesis in DIO Estrogen-Deficient Rats.

After metabolic analysis SHAM—vehicle, SHAM—amylin, OVX—vehicle and OVX—amylin rats (n=6 per group), were relocated to home caging and administered twice daily i.p. injections of bromodeoxyuridine (BrdU; 75 mg/kg) 12 hrs apart from days 5-12 post pump implantation. On day 14 animals were fully anesthetized with isoflurane and were perfused with saline followed by cold 4% paraformaldehyde. Brains were post-fixed overnight, cryoprotected in 30% sucrose for 24 h, then frozen on dry ice and stored at −80° C. until sectioning. Five series of 30 micron sections were cut on a freezing microtome.

Staining was performed on free-floating sections. Following PBS washes, sections were incubated in 50% formamide at 65° C. for two hours. DNA was denatured with 2N HCl for 30 min at 37° C. and then the acid was neutralized with 0.1M borate buffer. After washing with PBS, tissue was blocked in PBS/Triton/BSA, and then incubated overnight at 4° C. with rat monoclonal anti-BrdU (1:100, Accurate Chemical). The next day tissue was incubated with goat anti-rat Alexa-Fluor 488 secondary antibody (1:250, Molecular Probes, Eugene, Oreg.) in PBS/Triton for 2 hr in the dark. After washing with PBS, tissue was mounted immediately onto slides and coverslipped with Vectashield Prolong Gold antifade reagent (Molecular Probes) and stored at −20° C. Fluorescent images were captured and counted as described above.

Tissue from these rats was subsequently double labeled for BrdU along with NeuN, a marker for mature neurons, so that the type of cell could be accurately determined. The protocol was identical to that described above, except that sections were also incubated with mouse monoclonal primary anti-NeuN, (1:100, Chernicon International), and then with goat anti-mouse Alexa-Fluor 594 secondary antibody (1:250, Molecular Probes).

### Results.

c-Fos in the brainstem of OVX vs. SHAM rats, was first examined. No significant differences were found between amylin-treated groups (OVX=90±4 cells, SHAM=71±5 cells) or vehicle-treated groups (OVX=3±1 cells, SHAM=2±1 cells) in the AP, or between amylin-treated groups (OVX=30±1 cells, SHAM=34±2 cells) or vehicle-treated groups (OVX=4±1 cells, SHAM=1±0 cells) in the NTS. Thus, the differences in weight loss and food intake seen after amylin in OVX vs. SHAM rats were not correlated with short-term increases in amylin signaling, but rather are mediated by a longer-term mechanism developing over time.

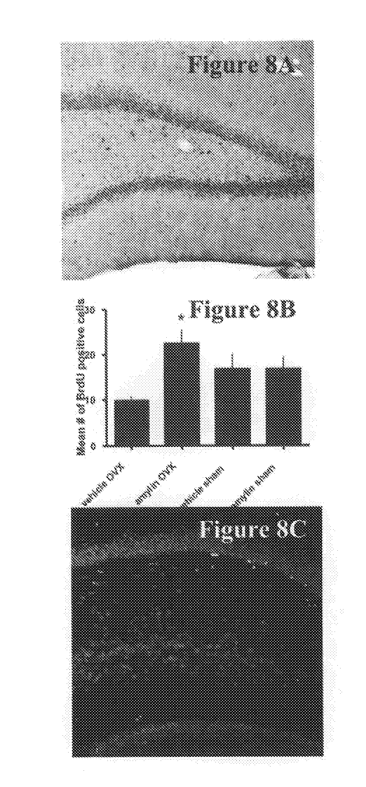

Ovariectomy is known to decrease neurogenesis in the hippocampus, a deficit that can be corrected with estrogen replacement (18). To explore the potential relationship between amylin, estrogen and neurogenesis, BrdU-immunoreactivity was measured within the area postrema (AP), the nucleus of the solitary tract (NTS) and the hippocampus, specific brain regions known to show spontaneous neurogenesis (19, 20). In the hippocampus—considered a positive control region—as expected, OVX treatment reduced neurogenesis below baseline and sustained infusion of amylin was able to restore it (p<0.05) (depicted quantitatively by graph in FIG. 8B; BrdU staining with DAB in hippocampous of an OVX/amylin-treated animal illustrated in FIG. 8A; BrdU/neuN staining in the hippocampus of a different OVX/amylin-treated animal illustrated in FIG. 8C).

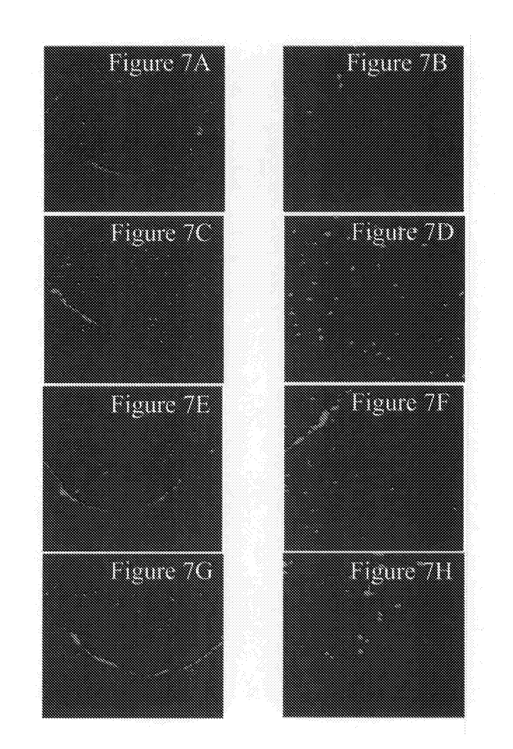

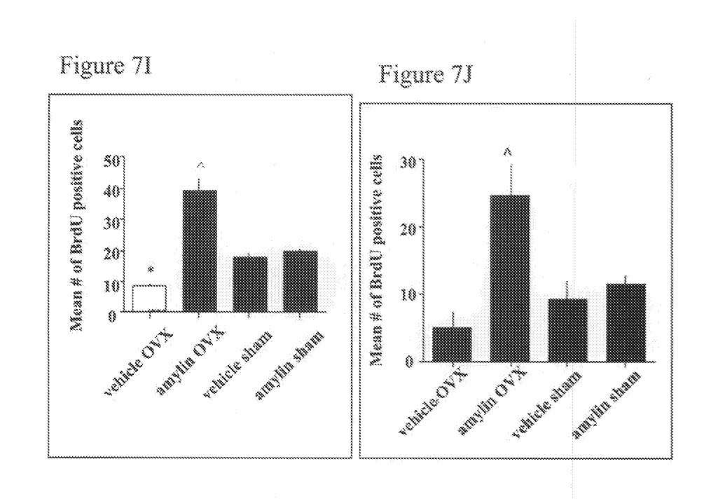

In the AP, compared to SHAM controls, OVX again significantly reduced neurogenesis in vehicle-treated animals (p<0.01), and amylin treatment was able to reverse this effect and even increase neurogenesis above baseline levels (p<0.001) in OVX animals (depicted quantitatively by graph in FIG. 7J; see, e.g., FIGS. 7A, 7C, 7E, and 7G). Additionally, double-labeling for BrdU+the neuron-specific marker NeuN showed that approximately 60% of the BrdU-positive cells in the AP were also positive for NeuN, and thus were mature neurons (not shown), while 38% of BrdU-positive neurons in the hippocampus were also positive for NeuN (FIGS. 8A-8C).

In the NTS, amylin significantly increased the number of BrdU-positive cells above that of OVX-vehicle, and both SHAM groups (p<0.05) (depicted quantitatively by graph in FIG. 71; see, e.g., FIGS. 7B, 7D, 7F, and 7H).

There were no other significant differences between groups in the NTS, and BrdU staining was not present in any other regions of the brain.

### Example 7

Invesitgation of a Role for Amylin-Dependent Increase in Leptin Sensitivity as a Mediator of Amylin's Improved Efficacy in Estrogen-Deficient Rats.

### Materials and Methods.

Amylin infusion to lean or obese, diabetic ZDF rats. Obese diabetic female ZDF rats (Charles River Laboratories) were maintained on 32% kcal/fat purified diet for approximately 6 weeks, and at 10 weeks of age were sorted into two weight-matched groups. One group was subjected to a bilateral ovariectomy (n=12) or SHAM surgery (n=12). Two weeks post-surgery rats were implanted with a single osmotic minipump (Alzet model 2ML4, Durect Corporation) delivering either vehicle or rat amylin (50 ug/kg/d) to half of each surgical group for 28 days. Lean (mean body weight 226±3 g) ZDF controls (Charles River) maintained on standard laboratory rodent chow (LM-485, 5% kcal/fat; Harlan Teklad) were sorted into weight-matched groups and implanted with a single osmotic minipump (Alzet model 2ML4, Durect Corporation) delivering either vehicle (n=9) or rat amylin (50 ug/kg/d; n=9) for 28 days. For both studies food intake and body weight were monitored weekly, and body composition was assessed at baseline and termination using NMR (Echo Medical Systems). After 28 days rats were euthanized by isoflurane overdose and cardiac blood collected.

Plasma metabolite analyses. Terminal plasma insulin (Crystal Chem Inc., Downer's Grove, Ill.), and leptin, total adiponectin and total ghrelin (Millipore, Billerica, Md.) levels were measured using commercially available ELISA. Plasma glucose, triglyceride, and total cholesterol levels were measured using an Olympus bioanalyzer (Olympus America Diagnostics, Center Valley, Pa.). Plasma amylin levels were measured using an internal IEMA.

### Results.

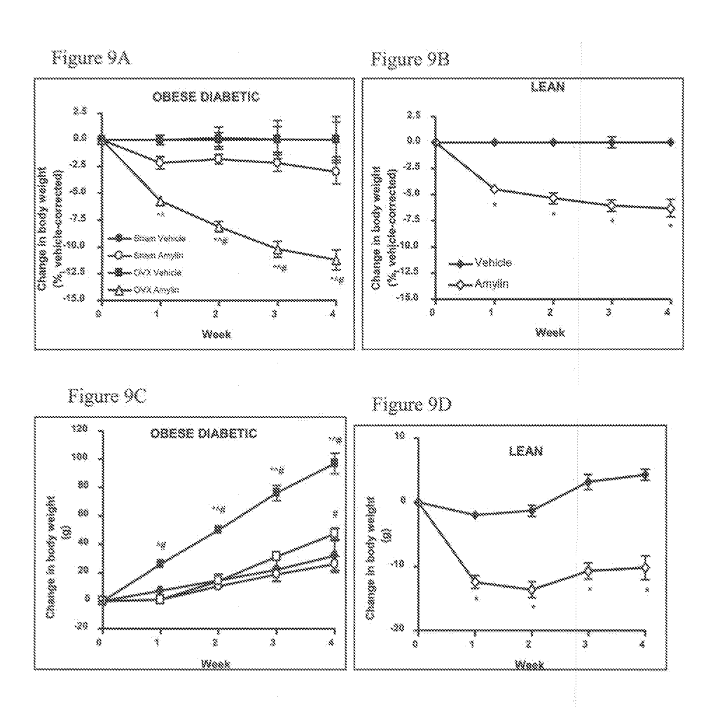

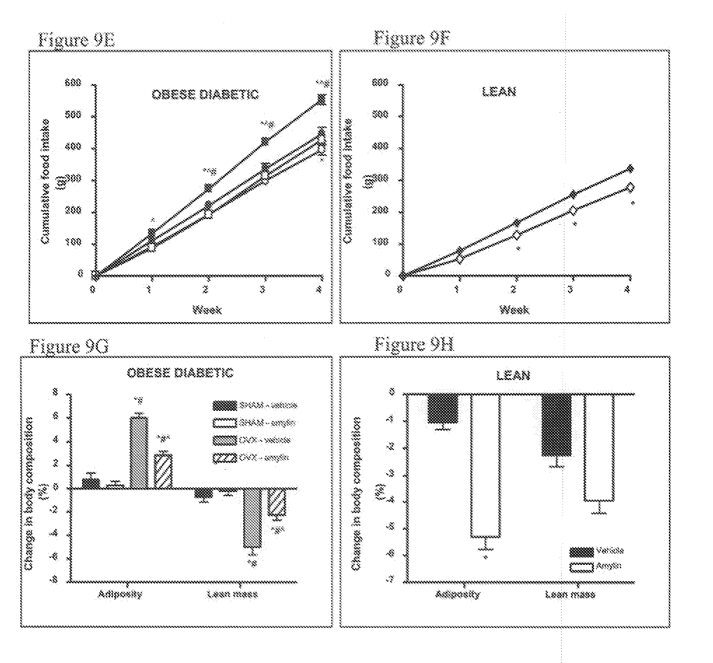

The OVX state is known to profoundly increase body weight as well as body fat mass and corresponding plasma leptin levels. To explore whether the exaggerated body weight loss in OVX rats was due at least in part to amylin sensitizing to elevations in peripheral leptin levels post-OVX surgery the effects of amylin infusion to ZDF rats which are obese and diabetic due to a non-functioning leptin receptor were examined. Sustained amylin infusion to SHAM ZDF rats did not significantly reduce body weight over 28 days (FIG. 9A). At the same dose, amylin infusion to OVX ZDF rats significantly reduced body weight relative to all other groups (FIG. 9A). Analysis of raw body weight change, however, revealed that the differences were due largely to the profound weight gain of ZDF rats post OVX surgery. Amylin pharmacology, therefore, did not so much induce actual weight loss, but rather significantly attenuated the gain in body weight of OVX ZDF rats (FIG. 9C). The drive to weight gain was evident even in amylin-treated OVX ZDF rats such that they exhibited significantly greater weight gain relative to SHAM—vehicle controls after 4 weeks (FIG. 9C). Changes in body weight were associated with inhibition of food intake (FIG. 9E). While amylin did not reduce body weight in SHAM ZDF rats, food intake after 4 weeks was inhibited relative to vehicle controls, however OVX ZDF—vehicle rats consumed more food than all other rats from weeks 1-4 (FIG. 9E). ZDF OVX rats also exhibited significantly increased fat mass that was attenuated by amylin administration, with corresponding reduction in lean mass (FIG. 9G).

As amylin infusion to SHAM ZDF rats appeared less effective at reducing body weight compared to DIO rats, the impact of amylin administration to lean rats on the same background strain as the ZDF rats was examined in order to rule out a strain-specific effect. In lean controls, amylin infusion significantly reduced body weight, reflected as true weight loss (FIGS. 9B and 9D), inhibited food intake (FIG. 9F) and specifically reduced fat mass (FIG. 9H), in a manner similar to that of DIO rats.

Analysis of plasma amylin levels revealed that hyperamylinemia in ZDF rats may contribute to the reduced efficacy of amylin in this model. Whereas all amylin-treated rats had similar circulating levels of amylin (Table 3), ZDF rats, irrespective of surgical status, exhibited significantly higher levels of amylin compared to lean controls (Table 2). Pharmacological levels of amylin were therefore ˜50-fold greater in lean rats, but in ZDF rats only a ˜12-fold increase in plasma amylin was evident. In ZDF and lean rats there was no effect of surgery or pharmacology on plasma glucose levels, and insulin was significantly reduced by amylin treatment in ZDF OVX and lean control rats (Table 3). Plasma triglycerides were reduced in OVX rats compared to SHAM ZDF controls and, unlike in lean animals where amylin infusion reduced plasma triglycerides, there was no effect of amylin (Table 3). Total cholesterol levels were reduced in SHAM ZDF rats, but were overall increased by OVX, with no change in lean animals (Table 3).

TABLE 2
Plasma parameters of DIO sham-operated (SHAM) controls, ovariectomized
(OVX) rats, or OVX plus estrogen (OVX + E) rats continuously infused
with either vehicle or rat amylin (50 μg/kg/d) for four weeks.

| | SHAM - | SHAM - | OVX - | OVX - | OVX + E - | OVX + E - |
|---|---|---|---|---|---|---|
| | vehicle | amylin | vehicle | amylin | vehicle | amylin |
| Glucose | 153 ± 4 | 139 ± 3 | 149 ± 7 | 155 ± 8 | 135 ± 7 | 131 ± 6 |
| (mg/dL) | | | | | | |
| Insulin | 1.1 ± 0.3 | 0.7 ± 0.1 | 1.6 ± 0.5 | 0.8 ± 0.1 | 0.8 ± 0.1 | 0.5 ± 0.1 |
| (ng/mL) | | | | | | |
| Triglycerides | 294 ± 59 | 161 ± 13 | 176 ± 24 | 162 ± 35 | 351 ± 93 | 138 ± 23* |
| (mg/dL) | | | | | | |
| Total | 89 ± 3 | 89 ± 6 | 100 ± 4 | 104 ± 8 | 116 ± 15 | 93 ± 7 |
| cholesterol | | | | | | |
| (mg/dL) | | | | | | |
| Leptin | 13.0 ± 2.3 | 6.0 ± 0.8* | 16.7 ± 2.2 | 9.0 ± 1.3* | 8.9 ± 1.4^{b} | 5.1 ± 0.9 |
| (ng/mL) | | | | | | |
| Adiponectin | 200 ± 22 | 185 ± 13 | 297 ± 29^{a} | 230 ± 35* | 175 ± 16^{b} | 155 ± 4 |
| (μg/mL) | | | | | | |
| Ghrelin | 1.9 ± 0.2 | 2.0 ± 0.1 | 1.4 ± 0.2 | 1.9 ± 0.3 | 2.0 ± 0.2 | 1.8 ± 0.1 |
| (ng/mL) | | | | | | |

*p < 0.05 vs. vehicle control within surgical group;
^{a}p < 0.05 vs. SHAM within drug treatment;
^{b}p < 0.05 vs. OVX within treatment.

TABLE 3
Plasma parameters of sham-operated (SHAM) or ovariectomized (OVX) ZDF rats, or lean controls,
continuously infused with either vehicle or rat amylin (50 μg/kg/d) for four weeks.

| | ZDF | ZDF | | | | |
|---|---|---|---|---|---|---|
| | SHAM - | SHAM - | ZDF OVX - | ZDF OVX - | Lean - | Lean - |
| | vehicle | amylin | vehicle | amylin | vehicle | amylin |
| Glucose | 267 ± 81 | 198 ± 60 | 155 ± 7 | 174 ± 15 | 143 ± 3 | 145 ± 3 |
| (mg/dL) | | | | | | |
| Insulin | 10.4 ± 1.9 | 8.1 ± 0.7 | 13.2 ± 2.2 | 7.5 ± 1.0* | 1.3 ± 0.1 | 0.6 ± 0.1* |
| (ng/mL) | | | | | | |
| Triglycerides | 1352 ± 171 | 1611 ± 183 | 619 ± 118^{a,b} | 584 ± 29^{a,b} | 202 ± 16 | 101 ± 19* |
| (mg/dL) | | | | | | |
| Total | 289 ± 29 | 209 ± 24* | 319 ± 18^{b} | 320 ± 25^{b} | 108 ± 3 | 106 ± 4 |
| cholesterol | | | | | | |
| (mg/dL) | | | | | | |
| Amylin | 292 ± 62 | 3452 ± 401* | 256 ± 43 | 3279 ± 430* | 60.9 ± 34^{a,c} | 3425 ± 392* |
| (pg/mL) | | | | | | |

*p < 0.05 vs. vehicle control within surgical group or lean group;
^{a}p < 0.05 vs. SHAM - vehicle;
^{b}p < 0.05 vs. SHAM - amylin;
^{c}p < 0.05 vs. OVX - vehicle.

### Discussion of Examples 3 Through 7

The impact of estrogen signaling on feeding behavior and overall energy homeostasis has been widely known for many years (1, 2). In addition to the specific actions of estrogen itself to attenuate food intake and reduce body weight, experimentally-induced estrogen deficiency results in hyperphagia and significant weight gain (1, 3, 21). OVX-mediated obesity is associated with tremendous gain in adipose tissue, particularly visceral fat (4), and the ability of estrogen to influence regional adipose deposition has been proposed (22). The mechanisms via which estrogen, or the absence of estrogen, mediate such pronounced effects on energy balance has been the subject of much investigation. The increased appreciation that the overarching control of body weight is regulated by multiple converging signaling pathways, incorporating putative short- and long-term signals of energy stores (23), implies that estrogen signaling may overlap with these systems at one or more levels to mediate its effects. The observations that estrogen status alters sensitivity to key regulators of energy balance such as NPY (10, 11), leptin (4, 7) and CCK (5, 6) confirms this hypothesis. While the evidence supporting a role for the pancreatic peptide amylin as a satiety signal continues to mount (13, 15), the impact of estrogen on amylin-mediated food intake and body weight regulation has not been explored.

If amylin-induced weight loss occurred independently of estrogen signaling, we would expect that body weight loss would be approximately equal between SHAM or OVX rats. However, we observed that at the same dose, amylin-mediated weight reduction was approximately 2-fold greater in OVX rats than in SHAM animals. Replacing estrogen to OVX rats clearly attenuated the ability of amylin to reduce body weight, in effect normalizing the amylin response. While OVX surgery itself induced significant body weight (˜40% more relative to SHAM controls) and fat mass gain, amylin-treated OVX rats did demonstrate “true” body weight loss, with negative weight change over the treatment period. In this respect, the impact of OVX on amylin signaling appears opposite than for other satiogenic neuropeptides such as CCK which exhibits reduced potency in OVX animals, and enhanced potency after estrogen administration (5, 6, 24). To explore potential mechanisms that might explain this unique phenomonon we focussed on three main areas: 1) peripheral metabolic consequences of amylin administration in SHAM and OVX DIO rats; 2) the contribution of enhanced amylin-dependent leptin action; and 3) amylin modulation of central responses to estrogen-deficiency, specifically the impact of OVX and subsequent amylin infusion on hindbrain neurogenesis.

Previous reports from our group and others have illustrated the potential for amylin maintain energy expenditure/metabolic rate in the face of hypophagia-driven weight loss (15, 25). We therefore characterized the metabolic component of enhanced amylin-mediated body weight loss in OVX DIO rats using indirect calorimetry. This analysis revealed a significantly enhanced pro-metabolic function for amylin in OVX animals. Relative to SHAM controls, OVX rats exhibited lower metabolic rate and increased RQ, indicating an enhanced metabolic efficiency, or ability to conserve and store more calories. This is in contrast to a previous study which reported only a trend for reduced energy expenditure in ad libitum fed OVX rats relative to SHAM controls after approximately 3 weeks, and no difference in RQ at any timepoint (26). These differences may be explained by the fact that we examined the consequence of OVX in obese rats, whereas Chen & Heiman, examined only periodic 24 hr calorimetry in normal weight animals (26). The metabolic consequences of estrogen deficiency may be more pronounced in states of obesity when OVX-induced positive energy balance may be more impactful relative to SHAMs than in leaner animals where all animals may be more prone to gaining weight. Indeed, a recent study has shown that leaner animals exhibit higher post-OVX feed efficiency (weight gain relative to food intake) in the first 3 weeks post surgery than do more obese rats, although this did not translate into a significantly increased rate of weight gain (27). Furthermore, OVX has been associated with reduced brown adipose tissue expression of uncoupling protein-1 mRNA, a common marker of metabolism, suggesting that metabolic rate was suppressed by OVX (28). Reduced metabolism and fat utilization in OVX rats could contribute significantly to the overall enhanced weight and adipose tissue gain in this model. As previously reported in DIO male rats (15), amylin agonism to SHAM rats maintained, but did not increase, metabolic rate and reduced RQ. There was also a significant increase in X-axis physical activity during the dark phase. In OVX rats however, amylin significantly increased, but did not fully normalize, metabolic rate and also reduced RQ and increased dark phase locomotor activity. Taken together it is apparent that the enhanced weight loss induced by amylin infusion to OVX rats is associated with a significant pro-metabolic effect. Comparisons to control OVX animals matched for food intake to amylin-treated OVX rats revealed that the weight loss can explain some, but not all, of these benefits. Yoked-fed OVX rats exhibited equally reduced RQ, but demonstrated a reduction in metabolic rate, a normal physiological response to caloric restriction. Therefore, while amylin-induced weight loss can account for benefits in substrate utilization, it cannot account for the apparent metabolic contribution of amylin agonism in this model.

There are strong links between estrogen signaling and leptin physiology (29). OVX-induced weight gain is associated with increased circulating leptin, likely due to the increased amount of fat, and has been proposed to induce a degree of central leptin resistance (10), although some reports have suggested that leptin can prevent/attenuate OVX-induced gain in weight and fat (26, 30). Amylin has been recently shown to act synergistically with leptin to profoundly reduce body weight in DIO rats. A dose of leptin that is ineffective at reducing body weight, when coupled with an effective dose of amylin (-5% body weight loss), results in ˜15% body weight loss in DIO rats (31, 32). The synergistic weight loss is fat-specific and reflective of recaptured leptin sensitivity in a previously leptin-resistant model: enhanced metabolic rate and increased fat utilization, and greater fat loss, relative to pair-fed controls (32). We explored the possibility that amylin reduced body weight to a greater extent in OVX rats by increasing sensitivity to endogenous leptin by infusing amylin to SHAM or OVX ZDF rats that exhibit an obesity syndrome due to a non-functioning leptin receptor. We hypothesized that as the entire signaling capacity of leptin is disrupted in this model any weight loss effects would be due solely to amylin agonism exclusive of any indirect leptin component. We observed that in ZDF rats, similar to DIO Sprague Dawley rats, amylin infusion was approximately 2-fold more potent at reducing body weight. In this model, “true” weight loss was not observed, rather amylin prevented the profound weight gain associated with OVX. Interestingly, amylin infusion to SHAM ZDF rats was also less efficacious compared to lean animals of the same background strain, possibly due to the innate hyperamylinemia of ZDF animals. Overall, the consistently observed increase in amylin efficacy for body weight loss in OVX animals with deficient leptin signaling implies that that this was not due to recruitment of the leptin pathway.

Beyond the peripheral consequences of enhanced amylin-mediated weight loss in states of estrogen deficiency, we explored some potential central mechanisms whereby these effects might be mediated. Adult neurogenesis in the hippocampus is believed to have possible importance for learning and memory (33), regulation of stress (34), and the positive effects of some antidepressant drugs (35). OVX is one of several experimental manipulations that has been found to decrease neurogenesis, along with stress (34), stress hormones such as glucocorticoids (36), aging (37) and sleep deprivation (36). Estrogen replacement can reverse this decrease in OVX animals (18, 38). However, previously published reports have been limited to observations within the hippocampus; the impact of estrogen on neurogenesis in other brain areas has not been reported. Recent studies have found spontaneous neurogenesis to be present in several hindbrain areas, including the AP and the nucleus tractus solitarius (NTS), similar to that seen in the hippocampus (20). We wanted to determine whether OVX would also attenuate spontaneous neurogenesis in the hindbrain, and whether amylin would reverse this effect. We observed that OVX decreased neurogenesis in the AP/NTS as well as the hippocampus, and that amylin was able to correct this deficit. Furthermore, in OVX rats, amylin appeared to increase neurogenesis beyond the level of that seen in SHAM animals after both vehicle and amylin treatment. Thus, it may be that the large increase in new neurons in the AP following amylin treatment led to the profound amylin-mediated weight loss in OVX animals, however this hypothesis remains to be tested. The observation that acute amylin-induced levels of c-Fos were not different between OVX and SHAM rats suggests that amylin's differing effects in the AP may be temporally or pharmacologically dependent. It will be important to determine whether if, after prolonged amylin treatment, the new neurons in the AP are amylin-responsive, or whether the enhanced weight loss seen after amylin in OVX animals can be attenuated if neurogenesis is prevented from occurring. Furthermore, a more direct link between amylin and estrogen signalling, such as may be confirmed by exploring the co-localization pattern of estrogen receptors and amylin-responsive neurons, in the AP as well as other hindbrain regions, remains to be determined. Colocalization of amylin-induced c-Fos with estrogen receptors in the AP, as well as examining whether an amylin antagonist would inhibit estrogen-induced reductions in food intake would help to establish whether such a direct link exists.

Exercise has been shown to increase hippocampal neurogenesis (37), whereas OVX has been linked with reductions in voluntary exercise (39). Caloric restriction also increases neurogenesis in the hippocampus (40). The extent to which caloric restriction and exercise can increase neurogenesis in the hindbrain has yet not yet been explored; likewise the role of weight loss per se on promoting hindbrain neurogenesis rather than direct amylin signaling cannot be delineated from these studies. DIO rats demonstrate significantly lower levels of neurogenesis in all the brains areas mentioned compared to lean rats (inventors' unpublished observations). Whether amylin- or weight-loss mediated changes in hindbrain neurogenesis are similarly affected by the predisposing weight status of the animal also remains unclear. Overall, the physiological consquences of increased hindbrain neuronal growth in the context of enhanced amylin-mediated weight loss in states of obesity-associated estrogen deficiency remain to be fully characterized.

Although the disclosure has been set forth in detail, one skilled in the art will appreciate that changes and modifications can be made without departing from the spirit and scope thereof.

Sequence Listing

SEQ ID NO: 1

KCNTATCATQRLANFLVHSSNNFGAILSSTNVGSNTY

Where ²Cys and ⁷Cys are linked by a disulfide bond; and ³⁷Tyr is optionally amidated.

SEQ ID NO: 2

CNTATCATQRLANFLVHSSNNFGAILSSTNVGSNTY

Where ¹Cys and ⁶Cys are optionally linked by a disulfide bond; and ³⁷Tyr is optionally amidated.

SEQ ID NO: 3

KCNTATCATQRLANFLIRSSNNLGAILSSTNVGSNTY

Where ²Cys and ⁷Cys are optionally linked by a disulfide bond; and ³⁷Tyr is optionally amidated.

SEQ ID NO: 4

KCNTATCATQRLANFLIRSSNNLGAVLSPTNVGSNTY

Where ²Cys and ⁷Cys are optionally linked by a disulfide bond; and ³⁷Tyr is optionally amidated.

SEQ ID NO: 5

KCNTATCATQRLANFLVHSSNNFGPILSSTNVGSNTY

Where ²Cys and ⁷Cys are optionally linked by a disulfide bond; and ³⁷Tyr is optionally amidated.

SEQ ID NO: 6

KCNTATCATQRLANFLVHSSNNFGAILPSTNVGSNTY

Where ²Cys and ⁷Cys are optionally linked by a disulfide bond; and ³⁷Tyr is optionally amidated.

SEQ ID NO: 7

KCNTATCATQRLANFLVHSSNNFGAILSPTNVGSNTY

Where ²Cys and ⁷Cys are optionally linked by a disulfide bond; and ³⁷Tyr is optionally amidated.

SEQ ID NO: 8

KCNTATCATQRLTNFLVRSSHNLGAALSPTDVGSNTY

Where ²Cys and ⁷Cys are optionally linked by a disulfide bond; and ³⁷Tyr is optionally amidated.

SEQ ID NO: 9

KCNTATCATQRLTNFLVHSSHNLGAALLPTDVGSNTY

Where ²Cys and ⁷Cys are optionally linked by a disulfide bond; and ³⁷Tyr is optionally amidated.

SEQ ID NO: 10

KCNTATCATQRLTNFLVHSSHNLGAALSPTDVGSNTY

Where ²Cys and ⁷Cys are optionally linked by a disulfide bond; and ³⁷Tyr is optionally amidated.

SEQ ID NO: 11

CNTATCATQRLTNFLVHSSHNLGAALSPTDVGSNTY

Where ¹Cys and ⁶Cys are optionally linked by a disulfide bond; and ³⁷Tyr is optionally amidated.

SEQ ID NO: 12

Lys Cys Asn Thr Ala Thr Cys Ala Thr Gln Arg Leu Ala Asn Phe Leu Arg His Ser Ser Asn Asn Phe Gly Thr Ile Leu Ser Ser Thr Asn Val Gly Ser Asp Thr Tyr

Where ²Cys and ⁷Cys are linked by a disulfide bond; and ³⁷Tyr is optionally amidated.

SEQ ID NO: 13

Lys Cys Asn Thr Ala Thr Cys Ala Thr Gln Arg Leu Ala Asn Phe Leu Ile Arg Ser Ser Asn Asn Leu Gly Ala Ile Leu Ser Pro Thr Asn Val Gly Ser Asn Thr Tyr

Where ²Cys and ⁷Cys are linked by a disulfide bond; and ³⁷Tyr is optionally amidated.

SEQ ID NO: 14

Lys Cys Asn Thr Ala Thr Cys Ala Thr Gln Arg Leu Ala Asn Phe Leu Val Arg Thr Ser Asn Asn Leu Gly Ala Ile Leu Ser Pro Thr Asn Val Gly Ser Asn Thr Tyr

Where ²Cys and ⁷Cys are linked by a disulfide bond; and ³⁷Tyr is optionally amidated.

SEQ ID NO: 15

Lys Cys Asn Thr Ala Thr Cys Ala Thr Gln Arg Leu Ala Asn Phe Leu Val Arg Ser Ser Asn Asn Leu Gly Pro Val Leu Pro Pro Thr Asn Val Gly Ser Asn Thr Tyr

Where ²Cys and ⁷Cys are linked by a disulfide bond; and ³⁷Tyr is optionally amidated.

SEQ ID NO: 16

Lys Cys Asn Thr Ala Thr Cys Ala Thr Gln Arg Leu Ala Asn Phe Leu Val Arg Ser Ser Asn Asn Leu Gly Pro Val Leu Pro Pro Thr Asn Val Gly Ser Asn Thr Tyr

Where ²Cys and ⁷Cys are linked by a disulfide bond; and ³⁷Tyr is optionally amidated.

SEQ ID NO: 17

Lys Cys Asn Thr Ala Thr Cys Ala Thr Gln Arg Leu Ala Asn Phe Leu Val Arg Ser Ser Asn Asn Leu Gly Pro Val Leu Ser Pro Thr Asn Val Gly Ser Asn Thr Tyr

Where ²Cys and ⁷Cys are linked by a disulfide bond; and ³⁷Tyr is optionally amidated.

SEQ ID NO: 18

Lys Cys Asn Thr Ala Thr Cys Ala Thr Gln Arg Leu Thr Asn Phe Leu Val Arg Ser Ser His Asn Leu Gly Ala Ala Leu Leu Pro Thr Asn Val Gly Ser Asn Thr Tyr

Where ²Cys and ⁷Cys are linked by a disulfide bond; and ³⁷Tyr is optionally amidated.

SEQ ID NO: 19

Lys Cys Asn Thr Ala Thr Cys Ala Thr Gln Arg Leu Thr Asn Phe Leu Val Arg Ser Ser Asn Asn Leu Gly Ala Ala Leu Leu Pro Thr Lys Val Gly Ser Asn Thr Tyr

Where ²Cys and ⁷Cys are linked by a disulfide bond; and ³⁷Tyr is optionally amidated.

SEQ ID NO: 20

KCNTATCATQRLANFLVHSSNNFGPILPPTNVGSNTY

Where ²Cys and ⁷Cys are optionally linked by a disulfide bond; and ³⁷Tyr is optionally amidated.

SEQ ID NO: 21

CNTATCATQRLANFLVHSSNNFGPILPPTNVGSNTY

Where ¹Cys and ⁶Cys are optionally linked by a disulfide bond; and ³⁷Tyr is optionally amidated.

SEQ ID NO: 22

KCNTATCATQRLANFLVRSSNNFGPILPPTNVGSNTY

Where ²Cys and ⁷Cys are optionally linked by a disulfide bond; and ³⁷Tyr is optionally amidated.

SEQ ID NO: 23

CNTATCATQRLANFLVRSSNNFGPVLPPTNVGSNTY

Where ¹Cys and ⁶Cys are optionally linked by a disulfide bond; and ³⁷Tyr is optionally amidated.

SEQ ID NO: 24

KCNTATCATQRLANFLVHSSNNFGPILPPTNVGSNTY

Where ²Cys and ⁷Cys are optionally linked by a disulfide bond; and ³⁷Tyr is optionally amidated.

SEQ ID NO: 25

KCNTATCATQRLANFLIHSSNNFGPILPPTNVGSNTY p Where ²Cys and ⁷Cys are optionally linked by a disulfide bond; and ³⁷Tyr is optionally amidated.

SEQ ID NO: 26

KCNTATCATQRLANFLVHSSNNLGPVLPPTNVGSNTY

Where ²Cys and ⁷Cys are optionally linked by a disulfide bond; and ³⁷Tyr is optionally amidated.

SEQ ID NO: 27

KCNTATCATQRLANFLVRSSNNLGPILPPTNVGSNTY

Where ²Cys and ⁷Cys are optionally linked by a disulfide bond; and ³⁷Tyr is optionally amidated.

SEQ ID NO: 28

KCNTATCATQRLANFLIHSSNNLGPILPPTNVGSNTY

Where ²Cys and ⁷Cys are optionally linked by a disulfide bond; and ³⁷Tyr is optionally amidated.

SEQ ID NO: 29

CNTATCATQRLANFLIHSSNNLGPILPPTNVGSNTY

Where ¹Cys and ⁶Cys are optionally linked by a disulfide bond; and ³⁷Tyr is optionally amidated.

SEQ ID NO: 30

KCNTATCATQRLANFLIRSSNNRGPVLPPTNVGSNTY

Where ²Cys and ⁷Cys are optionally linked by a disulfide bond; and ³⁷Tyr is optionally amidated.

SEQ ID NO: 31

KCNTATCATQRLTNFLVRSSHNLGPALPPTDVGSNTY

Where ²Cys and ⁷Cys are optionally linked by a disulfide bond; and ³⁷Tyr is optionally amidated.

SEQ ID NO: 32

KCNTATCATQRLANFLVRSSNNFGPILPSTNVGSNTY

Where ²Cys and ⁷Cys are optionally linked by a disulfide bond; and ³⁷Tyr is optionally amidated.

SEQ ID NO: 33

CNTATCATQRLANFLVRSSNNFGPILPSTNVGSNTY

Where ¹Cys and ⁶Cys are optionally linked by a disulfide bond; and ³⁷Tyr is optionally amidated.

SEQ ID NO: 34

KCNTATCATQRLANFLVRSSNNLGPILPSTNVGSNTY

Where ²Cys and ⁷Cys are optionally linked by a disulfide bond; and ³⁷Tyr is optionally amidated.

SEQ ID NO: 35

KCNTATCATQRLANFLVHSSNNLGPVLPSTNVGSNTY

Where ²Cys and ⁷Cys are optionally linked by a disulfide bond; and ³⁷Tyr is optionally amidated.

SEQ ID NO: 36

CNTATCATQRLANFLVHSSNNLGPVLPSTNVGSNTY

Where ¹Cys and ⁶Cys are optionally linked by a disulfide bond; and ³⁷Tyr is optionally amidated.

SEQ ID NO: 37

KCNTATCATQRLANFLVRSSNNLGPVLPSTNVGSNTY

Where ²Cys and ⁷Cys are optionally linked by a disulfide bond; and ³⁷Tyr is optionally amidated.

SEQ ID NO: 38

KCNTATCATQRLANFLVHSSNNFGPILPSTNVGSNTY

Where ²Cys and ⁷Cys are optionally linked by a disulfide bond; and ³⁷Tyr is optionally amidated.

SEQ ID NO: 39

KCNTATCATQRLTNFLVRSSHNLGAILPPTDVGSNTY

Where ²Cys and ⁷Cys are optionally linked by a disulfide bond; and ³⁷Tyr is optionally amidated.

SEQ ID NO:40

KCNTATCVLGKLSQELHRLQTYPRTNTGSNTY

Where ²Cys and ⁷Cys are optionally linked by a disulfide bond; and ³²Tyr is optionally amidated.

SEQ ID NO:41

KCNTATCVLGRLSQELHRLQTLPRTNTGSNTY

Where ²Cys and ⁷Cys are optionally linked by a disulfide bond; and ³²Tyr is optionally amidated.

SEQ ID NO:42

KCNTATCVLGRLSQELHRLQTYPPTNTGSNTY

Where ²Cys and ⁷Cys are optionally linked by a disulfide bond; and ³²Tyr is optionally amidated.

SEQ ID NO:43

KCNTATCVLGRLSQELHRLQTYPRTNVGSNTY

Where ²Cys and ⁷Cys are optionally linked by a disulfide bond; and ³²Tyr is optionally amidated.

SEQ ID NO:44

KCNTATCVLGRLSQELHRLQTLPPTNVGSNTY

Where ²Cys and ⁷Cys are optionally linked by a disulfide bond; and ³²Tyr is optionally amidated.

SEQ ID NO:45

KCNTATCVLGRLANFLHRLQTYPRTNTGSNTY

Where ²Cys and ⁷Cys are optionally linked by a disulfide bond; and ³²Tyr is optionally amidated.

SEQ ID NO:46

ACNTATCVLGRLSQELHRLQTYPRTNTGSNTY

Where ²Cys and ⁷Cys are optionally linked by a disulfide bond; and ³²Tyr is optionally amidated.

SEQ ID NO:47

KCNAATCVLGRLSQELHRLQTYPRTNTGSNTY

Where ²Cys and ⁷Cys are optionally linked by a disulfide bond; and ³²Tyr is optionally amidated.

SEQ ID NO:48

KCNTAACVLGRLSQELHRLQTYPRTNTGSNTY

Where ²Cys and ⁷Cys are optionally linked by a disulfide bond; and ³²Tyr is optionally amidated.

SEQ ID NO:49

CANLSTCVLGRLSQELHRLQTYPRTNTGSNTY

Where ²Cys and ⁷Cys are optionally linked by a disulfide bond; and ³²Tyr is optionally amidated.

SEQ ID NO:50

isocaproyl-STAVLGRLSQELHRLQTYPRTNTGSNTY

Where ²Cys and ⁷Cys are optionally linked by a disulfide bond; and ³²Tyr is optionally amidated.

SEQ ID NO:51

CSNASTCVLGRLSQELHRLQTYPRTNTGSNTY

Where ²Cys and ⁷Cys are optionally linked by a disulfide bond; and ³²Tyr is optionally amidated.

SEQ ID NO:52

CSNLATCVLGRLSQELHRLQTYPRTNTGSNTY

Where ²Cys and ⁷Cys are optionally linked by a disulfide bond; and ³²Tyr is optionally amidated.

SEQ ID NO:53

CSNLSACVLGRLSQELHRLQTYPRTNTGSNTY

Where ²Cys and ⁷Cys are optionally linked by a disulfide bond; and ³²Tyr is optionally amidated.

SEQ ID NO:54

KCNTATCVLGRLSQELHKLQTYPRTNTGSNTY

Where ²Cys and ⁷Cys are optionally linked by a disulfide bond; and ³²Tyr is optionally amidated.

SEQ ID NO:55

KCNTATCVLGRLSQELHRLQTYPRTNTGSGTP

Where ²Cys and ⁷Cys are optionally linked by a disulfide bond; and ³²Pro is optionally amidated.

SEQ ID NO:56

CSALSTCVLGRLSQELHRLQTYPRTNTGSNTY

Where ¹Cys and ⁶Cys are optionally linked by a disulfide bond; and ³²Tyr is optionally amidated.

SEQ ID NO:57

Ac-(Agy)SNLST(Agy)VLGRLSQELHRLQTYPRTNTGSNTY

Where the C-terminal Tyr is optionally amidated.

SEQ ID NO:58

Ac-K(Agy)NTAT(Agy)VLGRLSQELHRLQTYPRTNTGSNTY

Where the C-terminal Tyr is optionally amidated.

SEQ ID NO:59

Isocaproyl-STAVL(Aib)RLSQELRLQTYPRTNTGSGTP

Where the C-terminal Pro is optionally amidated.

SEQ ID NO:60

Isocaproyl-STAVLG[K(For)]LSQELH[K(For)]LQTYPRTNTGSGTP

Where the C-terminal Pro is optionally amidated.

SEQ ID NO:61

Isocaproyl-STAVL(Aib)[K(For)]LSQEL(Aib)[K(For)]LQTYPRTNTGSNTY

Where the C-terminal Tyr is optionally amidated.

SEQ ID NO:62

Isocaproyl-STAVL(Aib)[K(For)]LSQEL(Aib)[K(For)]LQTYPRTNVGSNTY

Where the C-terminal Tyr is optionally amidated.

SEQ ID NO:63

KCNTATCLLQQLQKLLQKLKQYPRTNTGSNTY Where ²Cys and ⁷Cys are optionally linked by a disulfide bond; and ³²Tyr is optionally amidated.

SEQ ID NO:64

KCNTASCVLGRLSQELHRLQTYPRTNTGSNTY Where ²Cys and ⁷Cys are optionally linked by a disulfide bond; and ³²Tyr is optionally amidated.

SEQ ID NO:65

KCNTAVCVLGRLSQELHRLQTYPRTNTGSNTY Where ²Cys and ⁷Cys are optionally linked by a disulfide bond; and ³²Tyr is optionally amidated.

SEQ ID NO:66

KCNTATCVLGRLSQELHRYPRTNTGSNTY Where ²Cys and ⁷Cys are optionally linked by a disulfide bond; and ²⁹Tyr is optionally amidated.

SEQ ID NO:67

KCNTATCVLGK(For)LSQELHK(For)LQTYPRTNTGSNTY Where ²Cys and ⁷Cys are optionally linked by a disulfide bond; and the C-terminal Tyr is optionally amidated.

SEQ ID NO:68

KCNTA(d-Thr)CVLGRLSQELHRLQTYPRTNTGSNTY Where ²Cys and ⁷Cys are optionally linked by a disulfide bond; and ³²Tyr is optionally amidated.

SEQ ID NO:69

KCNTA(dAh)CVLGRLSQELHRLQTYPRTNTGSNTY Where ²Cys and ⁷Cys are optionally linked by a disulfide bond; and ³²Tyr is optionally amidated.

SEQ ID NO:70

Ac-ACNTATCVLGRLSQELHK(PEG5000)LQTYPRTNTGSNTY Where ²Cys and ⁷Cys are optionally linked by a disulfide bond; and ³²Tyr is optionally amidated.

SEQ ID NO:71

KCNTATCVLGRLSQELHRLQTLQTYPRTNTGSNTY Where ²Cys and ⁷Cys are optionally linked by a disulfide bond; and ³⁵Tyr is optionally amidated.

SEQ ID NO:72

KCNTATCVLGRLSQELHRLQTLLQTYPRTNTGSNTY Where ²Cys and ⁷Cys are optionally linked by a disulfide bond; and ³⁵Tyr is optionally amidated.

SEQ ID NO:73

KCNTATCVLGKLSQELHKLQTYPRTNTGSNTY Where ²Cys and ⁷Cys are optionally linked by a disulfide bond; and ³²Tyr is optionally amidated.

SEQ ID NO:74

KCNTSTCVLGRLSQELHRLQTYPRTNTGSNTY Where ²Cys and ⁷Cys are optionally linked by a disulfide bond; and ³²Tyr is optionally amidated.

SEQ ID NO:75

KCNTATCATQRLSQELHRLQTYPRTNTGSNTY Where ²Cys and ⁷Cys are optionally linked by a disulfide bond; and ³²Tyr is optionally amidated.

SEQ ID NO:76

KCNTATCATQRLSQELHRLQTYPRTNVGSNTY Where ²Cys and ⁷Cys are optionally linked by a disulfide bond; and ³²Tyr is optionally amidated.

SEQ ID NO:77

KCNTSTCATQRLANELVRLQTYPRTNVGSNTY Where ²Cys and ⁷Cys are optionally linked by a disulfide bond; and ³²Tyr is optionally amidated.

SEQ ID NO:78

KCNTA(Hse)CVLGRLSQELHRLQTYPRTNTGSNTY Where ²Cys and ⁷Cys are optionally linked by a disulfide bond; and ³²Tyr is optionally amidated.

SEQ ID NO:79

KCNTA(Ahb)CVLGRLSQELHRLQTYPRTNTGSNTY Where ²Cys and ⁷Cys are optionally linked by a disulfide bond; and ³²Tyr is optionally amidated.

SEQ ID NO:80

KCNTA(Ahp)CVLGRLSQELHRLQTYPRTNTGSNTY Where ²Cys and ⁷Cys are optionally linked by a disulfide bond; and ³²Tyr is optionally amidated.

SEQ ID NO:81

KCNTAT(OPO3H2)CVLGRLSQELHRLQTYPRTNTGSNTY

Where ²Cys and ⁷Cys are optionally linked by a disulfide bond; and ³²Tyr is optionally amidated.

SEQ ID NO:82

KCNTATCVLG(Orn)LSQELH(Orn)LQTYPRTNTGSNTY

Where ²Cys and ⁷Cys are optionally linked by a disulfide bond; and ³²Tyr is optionally amidated.

SEQ ID NO:83

KCNTATCVLG(Cit)LSQELH(Cit)LQTYPRTNTGSNTY

Where ²Cys and ⁷Cys are optionally linked by a disulfide bond; and ³²Tyr is optionally amidated.

SEQ ID NO:84

KCNTATCVLG(homoK)LSQELH(homoK)LQTYPRTNTGSNTY

Where ²Cys and ⁷Cys are optionally linked by a disulfide bond; and ³²Tyr is optionally amidated.

SEQ ID NO:85

L-OctylglycineKCNTATCVLGRLSQELHRLQTYPRTNTGSNTY

Where ²Cys and ⁷Cys are optionally linked by a disulfide bond; and ³²Tyr is optionally amidated.

SEQ ID NO:86

N-3,6-dioxaoctanoyl-CNTATCVLGRLSQELHRLQTVPRTNTGSNTY

Where ²Cys and ⁷Cys are optionally linked by a disulfide bond; and ³²Tyr is optionally amidated.

SEQ ID NO:87

KCNTATCMLGRYTQDFHRLQTYPRTNTGSNTY

Where ²Cys and ⁷Cys are optionally linked by a disulfide bond; and ³²Tyr is optionally amidated.

SEQ ID NO:88

DSNLSTKVLGRLSQELHRLQTYPRTNTGSNTY

Where ³²Tyr is optionally amidated.

SEQ ID NO:89

KDNTATKVLGRLSQELHRLQTYPRTNTGSNTY

Where ³²Tyr is optionally amidated.

SEQ ID NO:90

CNTATCVLGRLSQELHRLQTYPRTNTGSNTY

Where ¹Cys and ⁶Cys are optionally linked by a disulfide bond; and ³¹Tyr is optionally amidated.

SEQ ID NO:91

KCNTATCVLGRLSQELHRLQTYPRTNTGSNTY(9Anc)

Where ²Cys and ⁷Cys are optionally linked by a disulfide bond; and ³²Tyr is optionally amidated.

SEQ ID NO:92

KCNTATCVLGRLSQELHRLQTYPRTNTGSNTY(L-octylglycine)

Where ²Cys and ⁷Cys are optionally linked by a disulfide bond; and ³²Tyr is optionally amidated.

SEQ ID NO:93

N-isocaproyl-KCNTATCVLGRLSQELHRLQTYPRTNTGSNTY

Where ²Cys and ⁷Cys are optionally linked by a disulfide bond; and ³²Tyr is optionally amidated.

SEQ ID NO:94

KCNTATCVLG(homoR)LSQELH(homoR)LQTYPRTNTGSNTY

Where ²Cys and ⁷Cys are optionally linked by a disulfide bond; and ³²Tyr is optionally amidated.

SEQ ID NO:95

FCNTATCVLGRLSQELHRLQTYPRTNTGSNTY

Where ²Cys and ⁷Cys are optionally linked by a disulfide bond; and ³²Tyr is optionally amidated.

SEQ ID NO:96

KCNTATCVLGRLSQELH(Cit)LQTYPRTNTGSNTY

Where ²Cys and ⁷Cys are optionally linked by a disulfide bond; and ³²Tyr is optionally amidated.

SEQ ID NO:97

KCNTATCVLGRLSQELH(Orn)LQTYPRTNTGSNTY

Where ²Cys and ⁷Cys are optionally linked by a disulfide bond; and ³²Tyr is optionally amidated.

SEQ ID NO:98

ICNTATCVLGRLSQELHRLQTYPRTNTGSNTY

Where ²Cys and ⁷Cys are optionally linked by a disulfide bond; and ³²Tyr is optionally amidated.

SEQ ID NO:99

1-Octylglycine-CNTATCVLGRLSQELHRLQTYPRTNTGSNTY

Where ²Cys and ⁷Cys are optionally linked by a disulfide bond; and ³²Tyr is optionally amidated.

SEQ ID NO:100

Isocaproyl-CNTATCVLGRLSQELHRLQTYPRTNTGSNTY

Where ²Cys and ⁷Cys are optionally linked by a disulfide bond; and ³²Tyr is optionally amidated.

SEQ ID NO:101

KCNTATCVLG(Cit)LSQELHRLQTYPRTNTGSNTY

Where ²Cys and ⁷Cys are optionally linked by a disulfide bond; and ³²Tyr is optionally amidated.

SEQ ID NO:102

KCNTATCVLGRLSQELHRLQTYPRTNTGSNTY(4ABU)

Where ²Cys and ⁷Cys are optionally linked by a disulfide bond; and ³²Tyr is optionally amidated.

SEQ ID NO:103

Isocaproyl-KCNTATCVLGRLSQELHRLQTYPRTNTGSNTY(4ABU)

Where ²Cys and ⁷Cys are optionally linked by a disulfide bond; and ³²Tyr is optionally amidated.

SEQ ID NO:104

KCNTSTCATQRLANELVRLQTYPRTNVGSEAF

Where ²Cys and ⁷Cys are optionally linked by a disulfide bond; and ³²Phe is optionally amidated.

SEQ ID NO:105

KCNTATCVLGRLSQELHRLQTYPTNVGSEAF

Where ²Cys and ⁷Cys are optionally linked by a disulfide bond; and ³²Phe is optionally amidated.

SEQ ID NO:106

KCNTATCVLGRLSRSLHRLQTYPRTNTGSNTY

Where ²Cys and ⁷Cys are optionally linked by a disulfide bond; and ³²Tyr is optionally amidated.

SEQ ID NO:107

KCNTATCVTHRLSQELHRLQTYPRTNTGSNTY

Where ²Cys and ⁷Cys are optionally linked by a disulfide bond; and ³²Tyr is optionally amidated.

SEQ ID NO:108

KCNTATCVLGRLADFLHRLQTYPRTNTGSNTY

Where ²Cys and ⁷Cys are optionally linked by a disulfide bond; and ³²Tyr is optionally amidated.

SEQ ID NO:109

CNTATCVLGRLSQELHRLQTYPRTNTGSNT

Where ¹Cys and ⁶Cys are optionally linked by a disulfide bond; and ³⁰Thr is optionally amidated.

SEQ ID NO:110

KCNTATCVLGRLSQELHRLQNFVPRTNTGSNTY

Where ²Cys and ⁷Cys are optionally linked by a disulfide bond; and ³²Tyr is optionally amidated.

SEQ ID NO:111

KCNTATCVLGRLSQELHRLQTYPRTNTGSETF

Where ²Cys and ⁷Cys are optionally linked by a disulfide bond; and ³²Phe is optionally amidated.

SEQ ID NO:112

ACDTATCVLGRLSQELHRLQTYPRTNTGSNTY

Where ²Cys and ⁷Cys are optionally linked by a disulfide bond; and ³²Tyr is optionally amidated.

SEQ ID NO:113

KCNTATCVLGRLSQELHRLQTYPRTNTGSKAF

Where ²Cys and ⁷Cys are optionally linked by a disulfide bond; and ³²Phe is optionally amidated.

SEQ ID NO:114

KCDTATCVTHRLAGLLSRSQTYPRTNTGSNTY

Where ²Cys and ⁷Cys are optionally linked by a disulfide bond; and ³²Tyr is optionally amidated.

SEQ ID NO:115

KCNTATCVLGRLADALHRLQTYPRTNTGSNTY

Where ²Cys and ⁷Cys are optionally linked by a disulfide bond; and ³²Tyr is optionally amidated.

SEQ ID NO:116

KCNTATCVLGRLAAFLHRLQTYPRTNTGSNTY

Where ²Cys and ⁷Cys are optionally linked by a disulfide bond; and ³²Tyr is optionally amidated.

SEQ ID NO:117

SCNTATCVLGRLADFLHRLQTYPRTNTGSNTY

Where ²Cys and ⁷Cys are optionally linked by a disulfide bond; and ³²Tyr is optionally amidated.

SEQ ID NO:118

KCNTATCVLGRLSQELHRLQTMPRTNTGSNTY

Where ²Cys and ⁷Cys are optionally linked by a disulfide bond; and ³²Tyr is optionally amidated.

SEQ ID NO:119

KCNTATCVLGRLSQELHRLQTVPRTNTGSNTY

Where ²Cys and ⁷Cys are optionally linked by a disulfide bond; and ³²Tyr is optionally amidated.

SEQ ID NO:120

KCNTATCVLGRLNEYLHRLQTYPRTNTGSNTY

Where ²Cys and ⁷Cys are optionally linked by a disulfide bond; and ³²Tyr is optionally amidated.

SEQ ID NO:121

SCNTATCVLGRLSQELHRLQTYPRTNTGSNTY

Where ²Cys and ⁷Cys are optionally linked by a disulfide bond; and ³²Tyr is optionally amidated.

SEQ ID NO:122

KCNTATCVLGRLTEFLHRLQTYPRTNTGSNTY

Where ²Cys and ⁷Cys are optionally linked by a disulfide bond; and ³²Tyr is optionally amidated.

SEQ ID NO:123

KCNTATCVLGRLAEFLHRLQTYPRTNTGSNTY

Where ²Cys and ⁷Cys are optionally linked by a disulfide bond; and ³²Tyr is optionally amidated.

SEQ ID NO:124

KCNTATCVLGRLTDYLHRLQTYPRTNTGSNTY

Where ²Cys and ⁷Cys are optionally linked by a disulfide bond; and ³²Tyr is optionally amidated.

SEQ ID NO:125

KCNTATCVLGRLAQFLHRLQTYPRTNTGSNTY

Where ²Cys and ⁷Cys are optionally linked by a disulfide bond; and ³²Tyr is optionally amidated.

SEQ ID NO:126

KCNTATCVLGRLADFLHRFQTFPRTNTGSNTY

Where ²Cys and ⁷Cys are optionally linked by a disulfide bond; and ³²Tyr is optionally amidated.

SEQ ID NO:127

KCNTATCVLGRLADFLHRFHTFPRTNTGSNTY

Where ²Cys and ⁷Cys are optionally linked by a disulfide bond; and ³²Tyr is optionally amidated.

SEQ ID NO:128

KCNTATCVLGRLADFLHRFQTFPRTNTGSGTP

Where ²Cys and ⁷Cys are optionally linked by a disulfide bond; and ³²Phe is optionally amidated.

SEQ ID NO:129

CNTATCVLGRLADFLHRLQTYPRTNTGSNTY

Where ²Cys and ⁷Cys are optionally linked by a disulfide bond; and ³²Tyr is optionally amidated.

SEQ ID NO:130

KCDTATCVLGRLSQELHRLQTYPRTNTGSNTY

Where ²Cys and ⁷Cys are optionally linked by a disulfide bond; and ³²Tyr is optionally amidated.

SEQ ID NO:131

KCNTATCVLGRLFDFLHRLQTYPRTNTGSNTY

Where ²Cys and ⁷Cys are optionally linked by a disulfide bond; and ³²Tyr is optionally amidated.

SEQ ID NO:132

KCNTATCVLGRLAAALHRLQTYPRTNTGSNTY

Where ²Cys and ⁷Cys are optionally linked by a disulfide bond; and ³²Tyr is optionally amidated.

SEQ ID NO:133

TCDTATCVLGRLSQELHRLQTYPRTNTGSNTY

Where ²Cys and ⁷Cys are optionally linked by a disulfide bond; and ³²Tyr is optionally amidated.

SEQ ID NO:134

CSNLSTCATQRLANELVRLQTYPRTNVGSNTY

Where ²Cys and ⁷Cys are optionally linked by a disulfide bond; and ³²Tyr is optionally amidated.

SEQ ID NO:135

KCNTATCATQRLANELVRLQTYPRTNVGSNTY

Where ²Cys and ⁷Cys are optionally linked by a disulfide bond; and ³²Tyr is optionally amidated.

SEQ ID NO:136

CSNLSTCVLGRLSQELHRLQTYPRTNTGSNTY

Where ²Cys and ⁷Cys are optionally linked by a disulfide bond; and ³²Tyr is optionally amidated.

SEQ ID NO:137

KCNTATCVLGRLSQELHRLQTYPRTNTGSNTY

Where ²Cys and ⁷Cys are optionally linked by a disulfide bond; and ³²Tyr is optionally amidated.

SEQ ID NO: 138

KCNTATCATQRLANFLVHSSNNFGAILPPTNVGSNTY

Where ²Cys and ⁷Cys are optionally linked by a disulfide bond; and ³⁷Tyr is optionally amidated.

SEQ ID NO: 139

KCNTATCATQRLANFLVHSSNNFGPILSPTNVGSNTY

Where ²Cys and ⁷Cys are optionally linked by a disulfide bond; and ³⁷Tyr is optionally amidated.

SEQ ID NO: 140

CGNLSTCMLGTYTQDFNKFHTFPQTAIGVGAP

Where ¹Cys and ⁷Cys are optionally linked by a disulfide bond; and ³²Pro is optionally amidated.

SEQ ID NO: 141

CSNLSTCVLGKLSQELHKLQTYPRTNTGSGTP

Where ¹Cys and ⁷Cys are optionally linked by a disulfide bond; and ³²Pro is optionally amidated.

SEQ ID NO: 142

HGEGTFTSDLSKQLEEEAVRLFIEWLKNGGPSSGAPPPSGGGKCNTATCVLGRLSQELH RLQTYPRTNTGSNTY

Where ⁴⁴Cys and ⁴⁹Cys are optionally linked by a disulfide bond; and ³²Pro is optionally amidated.

1-99. (canceled)

100. A method for treating estrogen deficiency in a mammal in need thereof comprising administering to the estrogen-deficient mammal a therapeutically effective amount of an amylin agonist compound.

101. The method of claim 100, wherein the amylin agonist compound is a compound of Formula (I), (II), (III), (IV), (VI), (VI), (VII), (VIII), (IX), or (X).

102. The method of claim 100, wherein the amylin agonist compound comprises the amino acid sequence of any one of SEQ ID NOs:1-139 or a pharmaceutically acceptable salt thereof

103. The method of claim 100, wherein the amylin agonist compound is linked to a peptide, a carbohydrate, a saccharide, a polyethylene glycol, albumin, a fatty acid, or a polyamino acid.

104. The method of claim 100, wherein the amylin agonist compound is pramlintide.

105. The method of claim 100, further comprising administering an effective amount of leptin or a leptin analog.

106. A method for treating obesity or overweight in an estrogen-deficient mammal comprising administering to the estrogen-deficient mammal a therapeutically effective amount of an amylin agonist compound to treat the obesity or overweight in the mammal.

107. The method of claim 106, wherein the amylin agonist compound is a compound of Formula (I), (II), (III), (IV), (VI), (VI), (VII), (VIII), (IX), or (X).

108. The method of claim 106, wherein the amylin agonist compound comprises the amino acid sequence of any one of SEQ ID NOs:1-139 or a pharmaceutically acceptable salt thereof

109. The method of claim 106, wherein the amylin agonist compound is pramlintide.

110. The method of claim 106, wherein the amylin agonist compound is linked to a peptide, carbohydrate, a saccharide, a polyethylene glycol, albumin, a fatty acid, or a polyamino acid.

111. The method of claim 106, further comprising administering an effective amount of leptin or a leptin analog.

112. A method for reducing weight in an estrogen-deficient mammal comprising administering to the estrogen-deficient mammal a therapeutically effective amount of an amylin agonist compound.

113. The method of claim 112, wherein the amylin agonist compound is a compound of Formula (I), (II), (III), (IV), (VI), (VI), (VII), (VIII), (IX), or (X).

114. The method of claim 112, wherein the amylin agonist compound comprises the amino acid sequence of any one of SEQ ID NOs:1-139 or a pharmaceutically acceptable salt thereof.

115. The method of claim 112, wherein the amylin agonist compound is pramlintide.

116. The method of claim 112, wherein the amylin agonist compound is linked to a peptide, a carbohydrate, a saccharide, a polyethylene glycol, albumin, a fatty acid, or a polyamino acid.

117. A method of increasing Bdnf levels in a mammal in need thereof comprising administering to the mammal a therapeutically effective amount of an amylin agonist compound to increase Bdnf levels in the mammal.

118. The method of claim 117, wherein the amylin agonist compound is a compound of Formula (I), (II), (III), (IV), (VI), (VI), (VII), (VIII), (IX), or (X).

119. The method of claim 117, wherein the amylin agonist compound is pramlintide.


SEQUENCE LISTING (190 sequences)

SEQ ID NO: 1 - protein, 37 aa, Homo sapiens [sequence omitted]

SEQ ID NO: 2 - protein, 36 aa, Artificial Sequence
  Description of Artificial Sequence Synthetic polypeptide
CNTATCATQRLANFLVHSSNNFGAILSSTNVGSNTY

SEQ ID NO: 3 - protein, 37 aa, Artificial Sequence
  Description of Artificial Sequence Synthetic polypeptide
KCNTATCATQRLANFLIRSSNNLGAILSSTNVGSNTY

SEQ ID NO: 4 - protein, 37 aa, Artificial Sequence
  Description of Artificial Sequence Synthetic polypeptide
KCNTATCATQRLANFLIRSSNNLGAVLSPTNVGSNTY

SEQ ID NO: 5 - protein, 37 aa, Artificial Sequence
  Description of Artificial Sequence Synthetic polypeptide
KCNTATCATQRLANFLVHSSNNFGPILSSTNVGSNTY

SEQ ID NO: 6 - protein, 37 aa, Artificial Sequence
  Description of Artificial Sequence Synthetic polypeptide
KCNTATCATQRLANFLVHSSNNFGAILPSTNVGSNTY

SEQ ID NO: 7 - protein, 37 aa, Artificial Sequence
  Description of Artificial Sequence Synthetic polypeptide
KCNTATCATQRLANFLVHSSNNFGAILSPTNVGSNTY

SEQ ID NO: 8 - protein, 37 aa, Artificial Sequence
  Description of Artificial Sequence Synthetic polypeptide
KCNTATCATQRLTNFLVRSSHNLGAALSPTDVGSNTY

SEQ ID NO: 9 - protein, 37 aa, Artificial Sequence
  Description of Artificial Sequence Synthetic polypeptide
KCNTATCATQRLTNFLVHSSHNLGAALLPTDVGSNTY

SEQ ID NO: 10 - protein, 37 aa, Artificial Sequence
  Description of Artificial Sequence Synthetic polypeptide
KCNTATCATQRLTNFLVHSSHNLGAALSPTDVGSNTY

SEQ ID NO: 11 - protein, 36 aa, Artificial Sequence
  Description of Artificial Sequence Synthetic polypeptide
CNTATCATQRLTNFLVHSSHNLGAALSPTDVGSNTY

SEQ ID NO: 12 - protein, 37 aa, Unknown
  Description of Unknown Monkey amylin sequence
KCNTATCATQRLANFLRHSSNNFGTILSSTNVGSDTY

SEQ ID NO: 13 - protein, 37 aa, Unknown
  Description of Unknown Cat amylin sequence
KCNTATCATQRLANFLIRSSNNLGAILSPTNVGSNTY

SEQ ID NO: 14 - protein, 37 aa, Canis sp.
  DISULFID (2)..(7)
  C-term optionally amidated
KCNTATCATQRLANFLVRTSNNLGAILSPTNVGSNTY

SEQ ID NO: 15 - protein, 37 aa, Rattus sp.
  DISULFID (2)..(7)
  C-term optionally amidated
KCNTATCATQRLANFLVRSSNNLGPVLPPTNVGSNTY

SEQ ID NO: 16 - protein, 37 aa, Mus sp.; identical to SEQ ID NO: 15
  DISULFID (2)..(7)
  C-term optionally amidated

SEQ ID NO: 17 - protein, 37 aa, Unknown
  Description of Unknown Hamster amylin sequence
KCNTATCATQRLANFLVRSSNNLGPVLSPTNVGSNTY

SEQ ID NO: 18 - protein, 37 aa, Unknown
  Description of Unknown Guinea pig amylin sequence
KCNTATCATQRLTNFLVRSSHNLGAALLPTNVGSNTY

SEQ ID NO: 19 - protein, 37 aa, Unknown
  Description of Unknown Degu amylin sequence
KCNTATCATQRLTNFLVRSSNNLGAALLPTKVGSNTY

SEQ ID NO: 20 - protein, 37 aa, Artificial Sequence
  Description of Artificial Sequence Synthetic polypeptide
KCNTATCATQRLANFLVHSSNNFGPILPPTNVGSNTY

SEQ ID NO: 21 - protein, 36 aa, Artificial Sequence
  Description of Artificial Sequence Synthetic polypeptide
CNTATCATQRLANFLVHSSNNFGPILPPTNVGSNTY

SEQ ID NO: 22 - protein, 37 aa, Artificial Sequence
  Description of Artificial Sequence Synthetic polypeptide
KCNTATCATQRLANFLVRSSNNFGPILPPTNVGSNTY

SEQ ID NO: 23 - protein, 36 aa, Artificial Sequence
  Description of Artificial Sequence Synthetic polypeptide
CNTATCATQRLANFLVRSSNNFGPILPPTNVGSNTY

SEQ ID NO: 24 - protein, 37 aa, Artificial Sequence
  Description of Artificial Sequence Synthetic polypeptide
KCNTATCATQRLANFLVHSSNNFGPVLPPTNVGSNTY

SEQ ID NO: 25 - protein, 37 aa, Artificial Sequence
  Description of Artificial Sequence Synthetic polypeptide
KCNTATCATQRLANFLIHSSNNFGPILPPTNVGSNTY

SEQ ID NO: 26 - protein, 37 aa, Artificial Sequence
  Description of Artificial Sequence Synthetic polypeptide
KCNTATCATQRLANFLVHSSNNLGPVLPPTNVGSNTY

SEQ ID NO: 27 - protein, 37 aa, Artificial Sequence
  Description of Artificial Sequence Synthetic polypeptide
KCNTATCATQRLANFLVRSSNNLGPILPPTNVGSNTY

SEQ ID NO: 28 - protein, 37 aa, Artificial Sequence
  Description of Artificial Sequence Synthetic polypeptide
KCNTATCATQRLANFLIHSSNNLGPILPPTNVGSNTY

SEQ ID NO: 29 - protein, 36 aa, Artificial Sequence
  Description of Artificial Sequence Synthetic polypeptide
CNTATCATQRLANFLIHSSNNLGPILPPTNVGSNTY

SEQ ID NO: 30 - protein, 37 aa, Artificial Sequence
  Description of Artificial Sequence Synthetic polypeptide
KCNTATCATQRLANFLIRSSNNLGPVLPPTNVGSNTY

SEQ ID NO: 31 - protein, 37 aa, Artificial Sequence
  Description of Artificial Sequence Synthetic polypeptide
KCNTATCATQRLTNFLVRSSHNLGPALPPTDVGSNTY

SEQ ID NO: 32 - protein, 37 aa, Artificial Sequence
  Description of Artificial Sequence Synthetic polypeptide
KCNTATCATQRLANFLVRSSNNFGPILPSTNVGSNTY

SEQ ID NO: 33 - protein, 36 aa, Artificial Sequence
  Description of Artificial Sequence Synthetic polypeptide
CNTATCATQRLANFLVRSSNNFGPILPSTNVGSNTY

SEQ ID NO: 34 - protein, 37 aa, Artificial Sequence
  Description of Artificial Sequence Synthetic polypeptide
KCNTATCATQRLANFLVRSSNNLGPILPSTNVGSNTY

SEQ ID NO: 35 - protein, 37 aa, Artificial Sequence
  Description of Artificial Sequence Synthetic polypeptide
KCNTATCATQRLANFLVHSSNNLGPVLPSTNVGSNTY

SEQ ID NO: 36 - protein, 36 aa, Artificial Sequence
  Description of Artificial Sequence Synthetic polypeptide
CNTATCATQRLANFLVHSSNNLGPVLPSTNVGSNTY

SEQ ID NO: 37 - protein, 37 aa, Artificial Sequence
  Description of Artificial Sequence Synthetic polypeptide
KCNTATCATQRLANFLVRSSNNLGPVLPSTNVGSNTY

SEQ ID NO: 38 - protein, 37 aa, Artificial Sequence
  Description of Artificial Sequence Synthetic polypeptide
KCNTATCATQRLANFLVHSSNNFGPILPSTNVGSNTY

SEQ ID NO: 39 - protein, 37 aa, Artificial Sequence
  Description of Artificial Sequence Synthetic polypeptide
KCNTATCATQRLTNFLVRSSHNLGAILPPTDVGSNTY

SEQ ID NO: 40 - protein, 32 aa, Artificial Sequence
  Description of Artificial Sequence Synthetic polypeptide
KCNTATCVLGKLSQELHRLQTYPRTNTGSNTY

SEQ ID NO: 41 - protein, 32 aa, Artificial Sequence
  Description of Artificial Sequence Synthetic polypeptide
KCNTATCVLGRLSQELHRLQTLPRTNTGSNTY

SEQ ID NO: 42 - protein, 32 aa, Artificial Sequence
  Description of Artificial Sequence Synthetic polypeptide
KCNTATCVLGRLSQELHRLQTYPPTNTGSNTY

SEQ ID NO: 43 - protein, 32 aa, Artificial Sequence
  Description of Artificial Sequence Synthetic polypeptide
KCNTATCVLGRLSQELHRLQTYPRTNVGSNTY

SEQ ID NO: 44 - protein, 32 aa, Artificial Sequence
  Description of Artificial Sequence Synthetic polypeptide
KCNTATCVLGRLSQELHRLQTLPPTNVGSNTY

SEQ ID NO: 45 - protein, 32 aa, Artificial Sequence
  Description of Artificial Sequence Synthetic polypeptide
KCNTATCVLGRLANFLHRLQTYPRTNTGSNTY

SEQ ID NO: 46 - protein, 32 aa, Artificial Sequence
  Description of Artificial Sequence Synthetic polypeptide
ACNTATCVLGRLSQELHRLQTYPRTNTGSNTY

SEQ ID NO: 47 - protein, 32 aa, Artificial Sequence
  Description of Artificial Sequence Synthetic polypeptide
KCNAATCVLGRLSQELHRLQTYPRTNTGSNTY

SEQ ID NO: 48 - protein, 32 aa, Artificial Sequence
  Description of Artificial Sequence Synthetic polypeptide
KCNTAACVLGRLSQELHRLQTYPRTNTGSNTY

SEQ ID NO: 49 - protein, 32 aa, Artificial Sequence
  Description of Artificial Sequence Synthetic polypeptide
CANLSTCVLGRLSQELHRLQTYPRTNTGSNTY

SEQ ID NO: 50 - protein, 28 aa, Artificial Sequence
  Description of Artificial Sequence Synthetic peptide
STAVLGRLSQELHRLQTYPRTNTGSNTY

SEQ ID NO: 51 - protein, 32 aa, Artificial Sequence
  Description of Artificial Sequence Synthetic polypeptide
CSNASTCVLGRLSQELHRLQTYPRTNTGSNTY

SEQ ID NO: 52 - protein, 32 aa, Artificial Sequence
  Description of Artificial Sequence Synthetic polypeptide
CSNLATCVLGRLSQELHRLQTYPRTNTGSNTY

SEQ ID NO: 53 - protein, 32 aa, Artificial Sequence
  Description of Artificial Sequence Synthetic polypeptide
CSNLSACVLGRLSQELHRLQTYPRTNTGSNTY

SEQ ID NO: 54 - protein, 32 aa, Artificial Sequence
  Description of Artificial Sequence Synthetic polypeptide
KCNTATCVLGRLSQELHKLQTYPRTNTGSNTY

SEQ ID NO: 55 - protein, 32 aa, Artificial Sequence
  Description of Artificial Sequence Synthetic polypeptide
KCNTATCVLGRLSQELHRLQTYPRTNTGSGTP

SEQ ID NO: 56 - protein, 32 aa, Artificial Sequence
  Description of Artificial Sequence Synthetic polypeptide
CSALSTCVLGRLSQELHRLQTYPRTNTGSNTY

SEQ ID NO: 57 - protein, 32 aa, Artificial Sequence
  Description of Artificial Sequence Synthetic polypeptide
XSNLSTXVLGRLSQELHRLQTYPRTNTGSNTY

SEQ ID NO: 58 - protein, 32 aa, Artificial Sequence
  Description of Artificial Sequence Synthetic polypeptide
KXNTATXVLGRLSQELHRLQTYPRTNTGSNTY

SEQ ID NO: 59 - protein, 27 aa, Artificial Sequence
  Description of Artificial Sequence Synthetic peptide
STAVLXRLSQELRLQTYPRTNTGSGTP

SEQ ID NO: 60 - protein, 28 aa, Artificial Sequence
  Description of Artificial Sequence Synthetic peptide
STAVLGKLSQELHKLQTYPRTNTGSGTP

SEQ ID NO: 61 - protein, 28 aa, Artificial Sequence
  Description of Artificial Sequence Synthetic peptide
STAVLXKLSQELXKLQTYPRTNTGSNTY

SEQ ID NO: 62 - protein, 28 aa, Artificial Sequence
  Description of Artificial Sequence Synthetic peptide
STAVLXKLSQELXKLQTYPRTNVGSNTY

SEQ ID NO: 63 - protein, 32 aa, Artificial Sequence
  Description of Artificial Sequence Synthetic polypeptide
KCNTATCLLQQLQKLLQKLKQYPRTNTGSNTY

SEQ ID NO: 64 - protein, 32 aa, Artificial Sequence
  Description of Artificial Sequence Synthetic polypeptide
KCNTASCVLGRLSQELHRLQTYPRTNTGSNTY

SEQ ID NO: 65 - protein, 32 aa, Artificial Sequence
  Description of Artificial Sequence Synthetic polypeptide
KCNTAVCVLGRLSQELHRLQTYPRTNTGSNTY

SEQ ID NO: 66 - protein, 29 aa, Artificial Sequence
  Description of Artificial Sequence Synthetic peptide
KCNTATCVLGRLSQELHRYPRTNTGSNTY

SEQ ID NO: 67 - protein, 32 aa, Artificial Sequence
  Description of Artificial Sequence Synthetic polypeptide
KCNTATCVLGKLSQELHKLQTYPRTNTGSNTY

SEQ ID NO: 68 - protein, 32 aa, Artificial Sequence
  Description of Artificial Sequence Synthetic polypeptide
KCNTATCVLGRLSQELHRLQTYPRTNTGSNTY

SEQ ID NO: 69 - protein, 32 aa, Artificial Sequence
  Description of Artificial Sequence Synthetic polypeptide
KCNTAXCVLGRLSQELHRLQTYPRTNTGSNTY

SEQ ID NO: 70 - protein, 32 aa, Artificial Sequence
  Description of Artificial Sequence Synthetic polypeptide
ACNTATCVLGRLSQELHKLQTYPRTNTGSNTY

SEQ ID NO: 71 - protein, 35 aa, Artificial Sequence
  Description of Artificial Sequence Synthetic polypeptide
KCNTATCVLGRLSQELHRLQTLQTYPRTNTGSNTY

SEQ ID NO: 72 - protein, 36 aa, Artificial Sequence
  Description of Artificial Sequence Synthetic polypeptide
KCNTATCVLGRLSQELHRLQTLLQTYPRTNTGSNTY

SEQ ID NO: 73 - protein, 32 aa, Artificial Sequence; identical to SEQ ID NO: 67
  Description of Artificial Sequence Synthetic polypeptide

SEQ ID NO: 74 - protein, 32 aa, Artificial Sequence
  Description of Artificial Sequence Synthetic polypeptide
KCNTSTCVLGRLSQELHRLQTYPRTNTGSNTY

SEQ ID NO: 75 - protein, 32 aa, Artificial Sequence
  Description of Artificial Sequence Synthetic polypeptide
KCNTATCATQRLSQELHRLQTYPRTNTGSNTY

SEQ ID NO: 76 - protein, 32 aa, Artificial Sequence
  Description of Artificial Sequence Synthetic polypeptide
KCNTATCATQRLSQELHRLQTYPRTNVGSNTY

SEQ ID NO: 77 - protein, 32 aa, Artificial Sequence
  Description of Artificial Sequence Synthetic polypeptide
KCNTSTCATQRLANELVRLQTYPRTNVGSNTY

SEQ ID NO: 78 - protein, 32 aa, Artificial Sequence; identical to SEQ ID NO: 69
  Description of Artificial Sequence Synthetic polypeptide

SEQ ID NO: 79 - protein, 32 aa, Artificial Sequence; identical to SEQ ID NO: 69
  Description of Artificial Sequence Synthetic polypeptide

SEQ ID NO: 80 - protein, 32 aa, Artificial Sequence; identical to SEQ ID NO: 69
  Description of Artificial Sequence Synthetic polypeptide

SEQ ID NO: 81 - protein, 32 aa, Artificial Sequence; identical to SEQ ID NO: 68
  Description of Artificial Sequence Synthetic polypeptide

SEQ ID NO: 82 - protein, 32 aa, Artificial Sequence
  Description of Artificial Sequence Synthetic polypeptide
KCNTATCVLGXLSQELHXLQTYPRTNTGSNTY

SEQ ID NO: 83 - protein, 32 aa, Artificial Sequence; identical to SEQ ID NO: 82
  Description of Artificial Sequence Synthetic polypeptide

SEQ ID NO: 84 - protein, 32 aa, Artificial Sequence; identical to SEQ ID NO: 67
  Description of Artificial Sequence Synthetic polypeptide

SEQ ID NO: 85 - protein, 33 aa, Artificial Sequence
  Description of Artificial Sequence Synthetic polypeptide
GKCNTATCVLGRLSQELHRLQTYPRTNTGSNTY

SEQ ID NO: 86 - protein, 31 aa, Artificial Sequence
  Description of Artificial Sequence Synthetic polypeptide
CNTATCVLGRLSQELHRLQTVPRTNTGSNTY

SEQ ID NO: 87 - protein, 32 aa, Artificial Sequence
  Description of Artificial Sequence Synthetic polypeptide
KCNTATCMLGRYTQDFHRLQTYPRTNTGSNTY

SEQ ID NO: 88 - protein, 32 aa, Artificial Sequence
  Description of Artificial Sequence Synthetic polypeptide
DSNLSTKVLGRLSQELHRLQTYPRTNTGSNTY

SEQ ID NO: 89 - protein, 32 aa, Artificial Sequence
  Description of Artificial Sequence Synthetic polypeptide
KDNTATKVLGRLSQELHRLQTYPRTNTGSNTY

SEQ ID NO: 90 - protein, 31 aa, Artificial Sequence
  Description of Artificial Sequence Synthetic polypeptide
CNTATCVLGRLSQELHRLQTYPRTNTGSNTY

SEQ ID NO: 91 - protein, 33 aa, Artificial Sequence
  Description of Artificial Sequence Synthetic polypeptide
KCNTATCVLGRLSQELHRLQTYPRTNTGSNTYX

SEQ ID NO: 92 - protein, 33 aa, Artificial Sequence
  Description of Artificial Sequence Synthetic polypeptide
KCNTATCVLGRLSQELHRLQTYPRTNTGSNTYG

SEQ ID NO: 93 - protein, 32 aa, Artificial Sequence; identical to SEQ ID NO: 68
  Description of Artificial Sequence Synthetic polypeptide

SEQ ID NO: 94 - protein, 32 aa, Artificial Sequence; identical to SEQ ID NO: 68
  Description of Artificial Sequence Synthetic polypeptide

SEQ ID NO: 95 - protein, 32 aa, Artificial Sequence
  Description of Artificial Sequence Synthetic polypeptide
FCNTATCVLGRLSQELHRLQTYPRTNTGSNTY

SEQ ID NO: 96 - protein, 32 aa, Artificial Sequence
  Description of Artificial Sequence Synthetic polypeptide
KCNTATCVLGRLSQELHXLQTYPRTNTGSNTY

SEQ ID NO: 97 - protein, 32 aa, Artificial Sequence; identical to SEQ ID NO: 96
  Description of Artificial Sequence Synthetic polypeptide

SEQ ID NO: 98 - protein, 32 aa, Artificial Sequence
  Description of Artificial Sequence Synthetic polypeptide
ICNTATCVLGRLSQELHRLQTYPRTNTGSNTY

SEQ ID NO: 99 - protein, 32 aa, Artificial Sequence
  Description of Artificial Sequence Synthetic polypeptide
GCNTATCVLGRLSQELHRLQTYPRTNTGSNTY

SEQ ID NO: 100 - protein, 31 aa, Artificial Sequence; identical to SEQ ID NO: 90
  Description of Artificial Sequence Synthetic polypeptide

SEQ ID NO: 101 - protein, 32 aa, Artificial Sequence
  Description of Artificial Sequence Synthetic polypeptide
KCNTATCVLGXLSQELHRLQTYPRTNTGSNTY

SEQ ID NO: 102 - protein, 33 aa, Artificial Sequence; identical to SEQ ID NO: 91
  Description of Artificial Sequence Synthetic polypeptide

SEQ ID NO: 103 - protein, 33 aa, Artificial Sequence; identical to SEQ ID NO: 91
  Description of Artificial Sequence Synthetic polypeptide

SEQ ID NO: 104 - protein, 32 aa, Artificial Sequence
  Description of Artificial Sequence Synthetic polypeptide
KCNTSTCATQRLANELVRLQTYPRTNVGSEAF

SEQ ID NO: 105 - protein, 31 aa, Artificial Sequence
  Description of Artificial Sequence Synthetic polypeptide
KCNTATCVLGRLSQELHRLQTYPTNVGSEAF

SEQ ID NO: 106 - protein, 32 aa, Artificial Sequence
  Description of Artificial Sequence Synthetic polypeptide
KCNTATCVLGRLSRSLHRLQTYPRTNTGSNTY

SEQ ID NO: 107 - protein, 32 aa, Artificial Sequence
  Description of Artificial Sequence Synthetic polypeptide
KCNTATCVTHRLSQELHRLQTYPRTNTGSNTY

SEQ ID NO: 108 - protein, 32 aa, Artificial Sequence
  Description of Artificial Sequence Synthetic polypeptide
KCNTATCVLGRLADFLHRLQTYPRTNTGSNTY

SEQ ID NO: 109 - protein, 30 aa, Artificial Sequence
  Description of Artificial Sequence Synthetic polypeptide
CNTATCVLGRLSQELHRLQTYPRTNTGSNT

SEQ ID NO: 110 - protein, 33 aa, Artificial Sequence
  Description of Artificial Sequence Synthetic polypeptide
KCNTATCVLGRLSQELHRLQNFVPRTNTGSNTY

SEQ ID NO: 111 - protein, 32 aa, Artificial Sequence
  Description of Artificial Sequence Synthetic polypeptide
KCNTATCVLGRLSQELHRLQTYPRTNTGSETF

SEQ ID NO: 112 - protein, 32 aa, Artificial Sequence
  Description of Artificial Sequence Synthetic polypeptide
ACDTATCVLGRLSQELHRLQTYPRTNTGSNTY

SEQ ID NO: 113 - protein, 32 aa, Artificial Sequence
  Description of Artificial Sequence Synthetic polypeptide
KCNTATCVLGRLSQELHRLQTYPRTNTGSKAF

SEQ ID NO: 114 - protein, 32 aa, Artificial Sequence
  Description of Artificial Sequence Synthetic polypeptide
KCDTATCVTHRLAGLLSRSQTYPRTNTGSNTY

SEQ ID NO: 115 - protein, 32 aa, Artificial Sequence
  Description of Artificial Sequence Synthetic polypeptide
KCNTATCVLGRLADALHRLQTYPRTNTGSNTY

SEQ ID NO: 116 - protein, 32 aa, Artificial Sequence
  Description of Artificial Sequence Synthetic polypeptide
KCNTATCVLGRLAAFLHRLQTYPRTNTGSNTY

SEQ ID NO: 117 - protein, 32 aa, Artificial Sequence
  Description of Artificial Sequence Synthetic polypeptide
SCNTATCVLGRLADFLHRLQTYPRTNTGSNTY

SEQ ID NO: 118 - protein, 32 aa, Artificial Sequence
  Description of Artificial Sequence Synthetic polypeptide
KCNTATCVLGRLSQELHRLQTMPRTNTGSNTY

SEQ ID NO: 119 - protein, 32 aa, Artificial Sequence
  Description of Artificial Sequence Synthetic polypeptide
KCNTATCVLGRLSQELHRLQTVPRTNTGSNTY

SEQ ID NO: 120 - protein, 32 aa, Artificial Sequence
  Description of Artificial Sequence Synthetic polypeptide
KCNTATCVLGRLNEYLHRLQTYPRTNTGSNTY

SEQ ID NO: 121 - protein, 32 aa, Artificial Sequence
  Description of Artificial Sequence Synthetic polypeptide
SCNTATCVLGRLSQELHRLQTYPRTNTGSNTY

SEQ ID NO: 122 - protein, 32 aa, Artificial Sequence
  Description of Artificial Sequence Synthetic polypeptide
KCNTATCVLGRLTEFLHRLQTYPRTNTGSNTY

SEQ ID NO: 123 - protein, 32 aa, Artificial Sequence
  Description of Artificial Sequence Synthetic polypeptide
KCNTATCVLGRLAEFLHRLQTYPRTNTGSNTY

SEQ ID NO: 124 - protein, 32 aa, Artificial Sequence
  Description of Artificial Sequence Synthetic polypeptide
KCNTATCVLGRLTDYLHRLQTYPRTNTGSNTY

SEQ ID NO: 125 - protein, 32 aa, Artificial Sequence
  Description of Artificial Sequence Synthetic polypeptide
KCNTATCVLGRLAQFLHRLQTYPRTNTGSNTY

SEQ ID NO: 126 - protein, 32 aa, Artificial Sequence
  Description of Artificial Sequence Synthetic polypeptide
KCNTATCVLGRLADFLHRFQTFPRTNTGSNTY

SEQ ID NO: 127 - protein, 32 aa, Artificial Sequence
  Description of Artificial Sequence Synthetic polypeptide
KCNTATCVLGRLADFLHRFHTFPRTNTGSNTY

SEQ ID NO: 128 - protein, 32 aa, Artificial Sequence
  Description of Artificial Sequence Synthetic polypeptide
KCNTATCVLGRLADFLHRFQTFPRTNTGSGTP

SEQ ID NO: 129 - protein, 31 aa, Artificial Sequence
  Description of Artificial Sequence Synthetic polypeptide
CNTATCVLGRLADFLHRLQTYPRTNTGSNTY

SEQ ID NO: 130 - protein, 32 aa, Artificial Sequence
  Description of Artificial Sequence Synthetic polypeptide
KCDTATCVLGRLSQELHRLQTYPRTNTGSNTY

SEQ ID NO: 131 - protein, 32 aa, Artificial Sequence
  Description of Artificial Sequence Synthetic polypeptide
KCNTATCVLGRLFDFLHRLQTYPRTNTGSNTY

SEQ ID NO: 132 - protein, 32 aa, Artificial Sequence
  Description of Artificial Sequence Synthetic polypeptide
KCNTATCVLGRLAAALHRLQTYPRTNTGSNTY

SEQ ID NO: 133 - protein, 32 aa, Artificial Sequence
  Description of Artificial Sequence Synthetic polypeptide
TCDTATCVLGRLSQELHRLQTYPRTNTGSNTY

SEQ ID NO: 134 - protein, 32 aa, Artificial Sequence
  Description of Artificial Sequence Synthetic polypeptide
CSNLSTCATQRLANELVRLQTYPRTNVGSNTY

SEQ ID NO: 135 - protein, 32 aa, Artificial Sequence
  Description of Artificial Sequence Synthetic polypeptide
KCNTATCATQRLANELVRLQTYPRTNVGSNTY

SEQ ID NO: 136 - protein, 32 aa, Artificial Sequence
  Description of Artificial Sequence Synthetic polypeptide
CSNLSTCVLGRLSQELHRLQTYPRTNTGSNTY

SEQ ID NO: 137 - protein, 32 aa, Artificial Sequence; identical to SEQ ID NO: 68
  Description of Artificial Sequence Synthetic polypeptide

SEQ ID NO: 138 - protein, 37 aa, Artificial Sequence
  Description of Artificial Sequence Synthetic polypeptide
KCNTATCATQRLANFLVHSSNNFGAILPPTNVGSNTY

SEQ ID NO: 139 - protein, 37 aa, Artificial Sequence
  Description of Artificial Sequence Synthetic polypeptide
KCNTATCATQRLANFLVHSSNNFGPILSPTNVGSNTY

SEQ ID NO: 140 - protein, 32 aa, Homo sapiens
  DISULFID (1)..(7)
  C-term optionally amidated
CGNLSTCMLGTYTQDFNKFHTFPQTAIGVGAP

SEQ ID NO: 141 - protein, 32 aa, Unknown
  Description of Unknown Salmon calcitonin sequence
CSNLSTCVLGKLSQELHKLQTYPRTNTGSGTP

SEQ ID NO: 142 - protein, 74 aa, Artificial Sequence
  Description of Artificial Sequence Synthetic polypeptide
HGEGTFTSDLSKQLEEEAVRLFIEWLKNGGPSSGAPPPSGGGKCNTATCVLGRLSQELHRLQTYPRTNTGSNTY

SEQ ID NO: 143 - protein, 37 aa, Artificial Sequence
  Description of Artificial Sequence Synthetic polypeptide
XCNTATCATQRLXNFLXXXXXNXGXXLXXTXVGSXTY

SEQ ID NO: 144 - protein, 37 aa, Artificial Sequence
  Description of Artificial Sequence Synthetic polypeptide
XXNTATXATQRLXNFLXXXXXNXGPXLPPTXVGSNTY

SEQ ID NO: 145 - protein, 37 aa, Artificial Sequence
  Description of Artificial Sequence Synthetic polypeptide
XXNTATXATQRLXNFLXXXXXNXGPXLPXTXVGSNTY

SEQ ID NO: 146 - protein, 37 aa, Artificial Sequence
  Description of Artificial Sequence Synthetic polypeptide
XXNTATXATQRLXNFLXXXXXNXGAXLPPTXVGSNTY

SEQ ID NO: 147 - protein, 37 aa, Artificial Sequence
  Description of Artificial Sequence Synthetic polypeptide
XXNTATXATQRLXNFLXXXXXNXGPXLXPTXVGSNTY

SEQ ID NO: 148 - protein, 32 aa, Artificial Sequence
  Description of Artificial Sequence Synthetic polypeptide
XCNTATCXXXRLXXXLXXLQTYPRTXVGSXTY

SEQ ID NO: 149 - protein, 6 aa, Artificial Sequence
  Description of Artificial Sequence Synthetic peptide
CNTATC

SEQ ID NO: 150 - protein, 6 aa, Artificial Sequence
  Description of Artificial Sequence Synthetic peptide
CATATC

SEQ ID NO: 151 - protein, 6 aa, Artificial Sequence
  Description of Artificial Sequence Synthetic peptide
CDTATC

SEQ ID NO: 152 - protein, 6 aa, Artificial Sequence
  Description of Artificial Sequence Synthetic peptide
CGTATC

SEQ ID NO: 153 - protein, 6 aa, Artificial Sequence
  Description of Artificial Sequence Synthetic peptide
CNAATC

SEQ ID NO: 154 - protein, 6 aa, Artificial Sequence
  Description of Artificial Sequence Synthetic peptide
CNTSTC

SEQ ID NO: 155 - protein, 6 aa, Artificial Sequence
  Description of Artificial Sequence Synthetic peptide
CNTATC

SEQ ID NO: 156 - protein, 6 aa, Artificial Sequence
  Description of Artificial Sequence Synthetic peptide
CNTASC

SEQ ID NO: 157 - protein, 6 aa, Artificial Sequence
  Description of Artificial Sequence Synthetic peptide
CNTAAC

SEQ ID NO: 158 - protein, 6 aa, Artificial Sequence
  Description of Artificial Sequence Synthetic peptide
CNTAVC

SEQ ID NO: 159 - protein, 6 aa, Artificial Sequence
  Description of Artificial Sequence Synthetic peptide
CNTAXC

SEQ ID NO: 160 - protein, 6 aa, Artificial Sequence
  Description of Artificial Sequence Synthetic peptide
CNTAXC

SEQ ID NO: 161 - protein, 6 aa, Artificial Sequence
  Description of Artificial Sequence Synthetic peptide
CNTAXC

SEQ ID NO: 162 - protein, 7 aa, Artificial Sequence
  Description of Artificial Sequence Synthetic peptide
CSNLSTC

SEQ ID NO: 163 - protein, 7 aa, Artificial Sequence
  Description of Artificial Sequence Synthetic peptide
CGNLSTC

SEQ ID NO: 164 - protein, 7 aa, Artificial Sequence
  Description of Artificial Sequence Synthetic peptide
CANLSTC

SEQ ID NO: 165 - protein, 7 aa, Artificial Sequence
  Description of Artificial Sequence Synthetic peptide
CSALSTC

SEQ ID NO: 166 - protein, 7 aa, Artificial Sequence
  Description of Artificial Sequence Synthetic peptide
CSNASTC

SEQ ID NO: 167 - protein, 7 aa, Artificial Sequence
  Description of Artificial Sequence Synthetic peptide
CSNLATC

SEQ ID NO: 168 - protein, 7 aa, Artificial Sequence
  Description of Artificial Sequence Synthetic peptide
CSNLSAC

SEQ ID NO: 169 - protein, 7 aa, Artificial Sequence
  Description of Artificial Sequence Synthetic peptide
KCNTATC

SEQ ID NO: 170 - protein, 15 aa, Artificial Sequence
  Description of Artificial Sequence Synthetic peptide
LLQQLQKLLQKLKQY

SEQ ID NO: 171 - protein, 28 aa, Artificial Sequence
  Description of Artificial Sequence Synthetic peptide
XXXXVLXXLSQXLXXLQTXPXTNTXXXX

SEQ ID NO: 172 - protein, 28 aa, Artificial Sequence
  Description of Artificial Sequence Synthetic peptide
XXXXXXXRXXXXXXXXXXXPXTNTXXXX

SEQ ID NO: 173 - protein, 8 aa, Artificial Sequence
  Description of Artificial Sequence Synthetic peptide
KSNFVPTN

SEQ ID NO: 174 - protein, 8 aa, Artificial Sequence
  Description of Artificial Sequence Synthetic peptide
SNFVPTNV

SEQ ID NO: 175 - protein, 32 aa, Artificial Sequence
  Description of Artificial Sequence Synthetic polypeptide
XXXXXXXXXXXXXXXXXXXXXXPXTNXGSXXX

SEQ ID NO: 176 - protein, 32 aa, Artificial Sequence
  Description of Artificial Sequence Synthetic polypeptide
XXXXXTXXXXXLXXXLXXXXXXPXTNXGSXXX

SEQ ID NO: 177 - protein, 4 aa, Artificial Sequence
  Description of Artificial Sequence Synthetic peptide
LQTY

SEQ ID NO: 178 - protein, 32 aa, Artificial Sequence; identical to SEQ ID NO: 175
  Description of Artificial Sequence Synthetic polypeptide

SEQ ID NO: 179 - protein, 146 aa, Mus sp.
  MOD_RES (28)..(28): Gln or not present
VPIQKVQDDTKTLIKTIVTRINDISHTQSVSSKQKVTGLDFIPGLHPILTLSKMDQTLAVYQQILTSMPSRNVIQISNDLENLRDLLHVLAFSKSCHLPQASGLETLESLGGVLEASGYSTEVVALSRLQGSLQDMLQQLDLSPGC

SEQ ID NO: 180 - protein, 146 aa, Sus sp.
VPIWRVQDDTKTLIKTIVTRISDISHMQSVSSKQRVTGLDFIPGLHPVLSLSKMDQTLAIYQQILTSLPSRNVIQISNDLENLRDLLHLLASSKSCPLPQARALETLESLGGVLEASLYSTEVVALSRLQGALQDMLRQLDLSPGC

SEQ ID NO: 181 - protein, 146 aa, Bos sp.
  MOD_RES (28)..(28): Gln or not present
VPICKVQDDTKTLIKTIVTRINDISHTQSVSSKQRVTGLDFIPGLHPLLSLSKMDQTLAIYQQILTSLPSRNVVQISNDLENLRDLLHLLAASKSCPLPQVRALESLESLGVVLEASLYSTEVVALSRLQGSLQDMLRQLDLSPGC

SEQ ID NO: 182 - protein, 146 aa, Homo sapiens
  MOD_RES (27)..(27): Thr or Ala
VPIQKVQDDTKTLIKTIVTRINDISHXQSVSSKQKVTGLDFIPGLHPILTLSKMDQTLAVYQQILTSMPSRNVIQISNDLENLRDLLHVLAFSKSCHLPWASGLETLDSLGGVLEASGYSTEVVALSRLQGSLQDMLWQLDLSPGC

SEQ ID NO: 183 - protein, 146 aa, Macaca mulatta
VPIQKVQSDTKTLIKTIVTRINDISHTQSVSSKQRVTGLDFIPGLHPVLTLSQMDQTLAIYQQILINLPSRNVIQISNDLENLRDLLHLLAFSKSCHLPLASGLETLESLGDVLEASLYSTEVVALSRLQGSLQDMLWQLDLSPGC

SEQ ID NO: 184 - protein, 146 aa, Rattus sp.
VPIHKVQDDTKTLIKTIVTRINDISHTQSVSARQRVTGLDFIPGLHPILSLSKMDQTLAVYQQILTSLPSQNVLQIAHDLENLRDLLHLLAFSKSCSLPQTRGLQKPESLDGVLEASLYSTEVVALSRLQGSLQDILQQLDLSPEC

SEQ ID NO: 185 - protein, 145 aa, Unknown
  Description of Unknown Platypus leptin sequence
ISIEKIQADTKTLTKTIITRIIQLSTQNGVSTDQRVSGLDFIPGNQQFQNLADMDQTLAVYQQILSSLPMPDRTQISNDLENLRSLFALLATLKNCPFTRSDGLDTMEIWGGIVEESLYSTEVVTLDRLRKSLKNIEKQLDHIQG

SEQ ID NO: 186 - protein, 164 aa, Artificial Sequence
  Description of Artificial Sequence Synthetic polypeptide
MRCILLYGFLCVWQHLYYSHPISIEKIQADTKTLTKTIITRIIQLSTQNGVSTDQRVSGLDFIPGNQQFQNLADMDQTLAVYQQILSSLPMPDRISNDLENLRSLFALLATLKNCPFTRSDGLDTMEIWGGIVEESLYSTEVVTLDRLRKSLKNIEKQLDHIQG

SEQ ID NO: 187 - protein, 147 aa, Artificial Sequence
  Description of Artificial Sequence Synthetic polypeptide
MVPIQKVQDDTKTLIKTIVTRINDISHTQSVSSKQKVTGLDFIPGLHPILTLSKMDQTLAVYQQILTSMPSRNVIQISNDLENLRDLLHVLAFSKSCHLPWASGLETLDSLGGVLEASGYSTEVVALSRLQGSLQDMLWQLDLSPGC

SEQ ID NO: 188 - protein, 379 aa, Artificial Sequence
  Description of Artificial Sequence Synthetic polypeptide
MEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKVPIQKVQDDTKTLIKTIVTRINDISHTQSVSSKQKVTGLDFIPGLHPILTLSKMDQTLAVYQQILTSMPSRNVIQISNDLENLRDLLHVLAFSKSCHLPWASGLETLDSLGGVLEASGYSTEVVALSRLQGSLQDMLWQLDLSPGC

SEQ ID NO: 189 - protein, 147 aa, Artificial Sequence
  Description of Artificial Sequence Synthetic polypeptide
MVPIQKVQDDTKTLIKTIVTRINDISHTQSVSSKQKVTGLDFIPGLHPILTLSKMDQTLAVYQQILTSMPSRNVIQISNDLENLRDLLHVLAFSKSCHLPQASGLETLDSLGGVLEASGYSTEVVALSRLQGSLQDMLQQLDLSPGC

SEQ ID NO: 190 - protein, 146 aa, Artificial Sequence
  Description of Artificial Sequence Synthetic polypeptide
VPIQKVQDDTKTLIKTIVTRINDISHTQSVSSKQKVTGLEFIPGLHPILTLSKMDQTLAVYQQILTSMPSRNVIQISNDLENLRDLLHVLAFSKSCHLPQASGLETLESLGGVLEASGYSTEVVALSRLQGSLQDMLQQLDLSPGC